(19) United States
(12) Patent Application Publication
(10) Pub. No.: US 2003/0003471 A1
(43) Pub. Date: Jan. 2, 2003

(54) cDNAs encoding polypeptides

(76) Inventors: Omolayo O. Famodu, Newark, DE (US); Guo-Hua Miao, Johnston, IA (US); Carl R. Simmons, Des Moines, IA (US); Zude Weng, Des Plaines, IL (US); Rebecca E. Cahoon, Wilmington, DE (US); Hajime Sakai, Wilmington, DE (US); Zhu Qun, Wilmington, DE (US); Catherine J. Thorpe, Cambridgeshire (GB); Gary M. Fader, Landenberg, PA (US); Bailin Li, Hockessin, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/078,770

(22) Filed: Feb. 19, 2002

Related U.S. Application Data
(62) Division of application No. 09/614,188, filed on Jul. 11, 2000.
(60) Provisional application No. 60/143,410, filed on Jul. 12, 1999.
(60) Provisional application No. 60/143,409, filed on Jul. 12, 1999.
(60) Provisional application No. 60/153,534, filed on Sep. 13, 1999.
(60) Provisional application No. 60/143,400, filed on Jul. 12, 1999.
(60) Provisional application No. 60/161,223, filed on Oct. 22, 1999.
(60) Provisional application No. 60/159,878, filed on Oct. 15, 1999.
(60) Provisional application No. 60/157,401, filed on Oct. 1, 1999.

(51) Int. Cl. C12Q 1/68; C07H 21/04; C12N 9/00; C12N 1/21; C12P 21/02; C12N 5/04; C12N 5/06; C12N 1/18; C12N 7/00

(52) U.S. Cl. 435/6; 435/69.1; 435/325; 435/410; 435/252.3; 435/254.2; 435/235.1; 435/183; 536/23.2

(57) ABSTRACT

This invention relates to an isolated nucleic acid fragment encoding a phospholipase D. The invention also relates to the construction of a chimeric gene encoding all or a substantial portion of the phospholipase D, in sense or antisense orientation, wherein expression of the chimeric gene results in production of altered levels of the phospholipase D in a transformed host cell.

This application claims the benefit of U.S. Provisional Application No. 60/143,410, filed Jul. 12, 1999; U.S. Provisional Application No. 60/143,409, filed Jul. 12, 1999; U.S. Provisional Application No. 60/153,534, filed Sep. 13, 1999; U.S. Provisional Application No. 60/143,400, filed Jul. 12, 1999; U.S. Provisional Application No. 60/161,223, filed Oct. 22, 1999; U.S. Provisional Application No. 60/159,878, filed Oct. 15, 1999; and U.S. Provisional Application No. 60/157,401, filed Oct. 01, 1999, all of which are incorporated herein by reference.

### FIELD OF THE INVENTION

This invention is in the field of plant molecular biology. More specifically, it relates to nucleic acid sequences, the amino acids sequences encoded by such nucleic acids, and methods for modulating their expression in plants.

### BACKGROUND OF THE INVENTION

Reactive oxygen metabolites are produced as a response to pathogen attack in most organisms including bacteria, mammals and plants. Superoxide and hydrogen peroxide are generated by an NADPH-dependent oxidase. In humans this plasma membrane oxidase is formed of two subunits gp91^{phox} and p22^{phox} which act together with three cytosolic proteins p40^{phox}, p47^{phox} and p67^{phox} to form an active complex. An Arabidopsis thaliana gene encoding a respiratory burst oxidase homolog A (RbohA) with similarity to the human gp91^{phox} but also containing an amino-terminal domain with two calcium binding motifs has been described. The predicted amino acid sequence from this Arabidopsis thaliana gene contains binding sites and transmembrane domains which are conserved with the rice RbohA (Keller, T. et al. (1998) Plant Cell 10:255-266). At least 6 different Arabidopsis thaliana homologs, named RbohA, RbohB, RbohC, RbohD, RbohE, and RbohF, have been identified for the human gp91^{phox} (Torres et al. (1998) Plant J. 14:365-370).

There are multiple, possibly redundant or synergistic pathways in response to a pathogen attack. Understanding the genes involved will allow the study of stress response and the engineering of plants with stress and disease resistance.

Transfer RNA from all organisms typically contains several modified nucleosides, in addition to the standard guanosine, adenosine, cytidine, and uridine. These modified bases are important for tRNA folding and function. One group, 5-methylaminomethyl-2-thiouridylate, is found in the “wobble position” of the tRNA anticodon sequence. The modification is apparently important for the stabilization of tRNA pairing to the codon. Mutations inhibiting the base modification lead to loss of translational fidelity (Hagervall and Bjork (1984) Mol. Gen. Genet. 196:194-200). The enzyme that performs this modification is tRNA (5-methylaminomethyl-2-thiouridylate)-methyltransferase, also called tRNA-mnm⁵s²U-MT. Mutations in this enzyme can adversely affect translational regulation and can lead to lethality. Due to the lethal phenotype found in mutant genes, these are potential targets for herbicide treatment in plants, thus they will be useful for herbicide discovery and design.

Cytosine methylation is the most common modification of DNA found in nature. Cytosine methylation has been implicated in the control of many cellular processes including development, DNA repair, chromatin organization, transcription, recombination and replication. Cytosine 5-methyltransferase has been proposed to play a role in general biological processes such as cellular aging (Tollefsbol et al. (1993) Med. Hypotheses 41:83-92), carcinogenesis (Jones et al. (1990) Adv. Cancer Res. 54:1-23), human genetic diseases (Cooper et al. (1988) Hum. Genet. 78:151-155), and evolution (Sved et al. (1990) Proc. Natl. Acad. Sci. U.S.A. 87:4692-4696).

Another type of DNA methylation protein is chromomethylase. Eight different chromometylases have been identified in Arabidopsis thaliana (Henikoffet al. (1998) Genetics 149:307-318). These proteins have common chromodomains that are thought to mediate protein-protein interactions between various chromatin molecules. Chromomethylase may also be involved in controlling many cellular processes.

There is a great deal of interest in identifying the genes that encode proteins involved in DNA methylation in plants. These genes may be used in plant cells to control the cell development, transcription and DNA replication. Accordingly, the availability of nucleic acid sequences encoding all or a substantial portion of a DNA methyltransferase would facilitate studies to better understand DNA methylation in plants and provide genetic tools to inhibit or otherwise alter DNA methyltransferase activity which in turn could provide mechanisms to control cell development, transcription, DNA replication and other cellular processes in plant cells.

Phospholipase D (PLD; EC 3.1.4.4) catalyzes the breakdown of glycerophospholipids to produce choline and a phosphatidate. Originally considered to exist only in plants, PLDs also have been found in mammals and microorganisms. These enzymes have been proposed to play important roles in transmembrane signaling, vesicle traffic, and responses to internal and external stress. The first identified PLD (now called PLD-alpha) does not need polyphosphoinositide as a cofactor and shows higher activity in the presence of millimolar calcium concentrations. Two other PLDs identified in Arabidopsis thaliana (PLD-beta and PLD-gamma) require polyphosphoinositide as a cofactor and require microgram amounts of calcium for proper activity (Pappan et al. (1997) J. Biol. Chem. 272:7048-7054). These Arabidopsis thaliana PLDs have been further characterized and shown to have different biochemical properties. PLD-alpha and PLD-gamma fractionate with the plasma membrane, mitochondria, clathrin coated vesicles and intracellular membranes from Arabidopsis thaliana leaves. PLD-gamma is also found in the nuclear fraction while the amount of PLD-beta present makes it difficult to detect in subcellular fractions.

Genes encoding PLD-alpha from corn and rice have been previously identified (Ueki et al. (1995) Plant Cell. Physiol. 36:903-914). Genes encoding PLD-beta and PLD-gamma have only been identified in Arabidopsis thaliana. Identification of the genes encoding PLD-alpha in soybean and wheat and PLD-gamma in corn and soybean will enable the study of membrane signaling and stress response in agriculturally important crops. Lysophospholipids are incorporated within wheat starch granules during starch biosynthesis and phospholipase is implicated in the formation of lysophospholipid from phosphatidylcholine. Thus, manipulation of this biosynthetic pathway could enable the starch lipid content to be altered, generating starches with novel functional properties.

In eukaryotes transcription initiation requires the action of several proteins acting in concert to initiate mRNA production. Two cis-acting regions of DNA have been identified that bind transcription initiation proteins. The first binding site, located approximately 25-30 bp upstream of the transcription initiation site, is termed the “TATA box”. The second region of DNA required for transcription initiation is the upstream activation site (UAS) or enhancer region. This region of DNA is somewhat distal from the TATA box. During transcription initiation, RNA polymerase II is directed to the TATA box by general transcription factors. Transcription activators, which have both a DNA binding domain and an activation domain, bind to the UAS region and stimulate transcription initiation by physically interacting with the general transcription factors and RNA polymerase. Direct physical interactions have been demonstrated between activators and general transcription factors in vitro (Triezenberg et al. (1988) Gene Dev. 2:718-729; Stringer et al. (1990) Nature 345:783-786; Lin et al. (1991) Nature 353:569-571; Xiao et al. (1994) Mol. Cell. Biol. 14:7013-7024). One general transcription factor, TFIIF, has been shown to bind to RNA polymerase II and with the help of TFIIB, recruit RNA polymerase II to the initiation complex. Transcription factor TFIIF is one of the larger initiation factors, being composed of a tetramer consisting of two large alpha subunits and two small beta subunits (Gong et al. (1995) Nucleic Acids Res. 23:1182-1186).

It is thought that adaptor proteins serve to mediate the interaction between transcriptional activators and general transcription factors. Functional and physical interactions have also been demonstrated between the activators and various transcription adaptors. These transcription adaptors do not normally bind directly to DNA, but they can “bridge” the interaction between transcription activators and general transcription factors (Pugh and Tjian (1990) Cell 61:1187-1197; Kelleher et al. (1990) Cell 61:1209-1215; Berger et al. (1990) Cell 61:1199-1208).

Accordingly, the availability of nucleic acid sequences encoding all or a substantial portion of TFIIF alpha and/or beta subunits will facilitate studies to better understand transcription initiation in plants and ultimately will provide methods to engineer mechanisms to control transcription.

Aminoacyl-tRNA synthetases ensure the fidelity of protein biosynthesis by aminoacetylating tRNAs. There are at least 20 different aminoacyl-tRNA synthetases (one per amino acid). The first asparaginyl-tRNA synthetase gene from a higher plant (plants other than yeast) was identified in Arabidopsis thaliana chromosome IV (Aubourg et al. (1998) Biochim. Biophys. Acta 1398:225-231). A cDNA encoding Lupinus luteus Glutaminyl-tRNA synthetase has been characterized (NCBI General Identifier No. 3915866). Identification of aminoacyl-tRNA synthetases in other plants will be useful to develop herbicide-resistant plants and for the discovery and design of new herbicides.

Plant defenses are activated by an interaction between the plant resistance (R) gene and the pathogen avirulence (avr) gene. The precise mode of interaction between R and avr has not been elucidated to date. The cDNAs encoding R genes from several monocot and dicot species have been identified. The mechanism of transduction of the R gene signal has been studied using screens for mutations that affect disease resistance or that affect specific defense responses and using the yeast two hybrid system. These analyses have resulted in the idea that the R gene transduction pathways are highly branched (Innes (1998) Curr. Opin. Plant Biol. 1:229-304). Using a mutational approach, a recessive mutation called eds1 (enhanced disase susceptibility 1) was identified in Arabidopsis thaliana which abolishes the resistance to Peronospora parasitica in the Wassilewskija (Ws-0) background (Parker et al. (1996) Plant Cell 8:2033-2046). The EDS1 protein was shown to be indispensable for the function of the major class of R genes and contains a C-terminal region with similarities to eukaryotic lipases (Falk, et al. (1999) Proc. Natl. Acad. Sci. USA 96:3292-3297). Identification of EDS1 in other plants such as the rice, soybean, and wheat disclosed herein will allow the study of the transduction mechanism.

Adaptins are components of the complexes which link clathrin to receptors in coated vesicles. Clathrin-associated protein complexes are believed to interact with the cytoplasmic tails of membrane proteins leading to their selection and concentration. The plasma membrane adaptor (AP2) is a heterologous tetrameric complex composed of two large chains (alpha adaptin and beta adaptin), a medium chain (AP50), and a small chain (AP17). This adaptor complex is a component of the coat surrounding the cytoplasmic face of the coated vesicles in the plasma membrane. The cDNAs encoding two alpha adaptins have been isolated from mouse brain (Robinson (1989) J. Cell. Biol. 108:833-842) and a cDNA clone (Accession No. AF009631) encoding a protein homologous to the the micro-adaptins of clathrin-coated vesicle adaptor complexes has been identified in Arabidopsis thaliana. There are two beta adaptin subtypes, beta adaptin and beta′ adaptin. The beta′ adaptins from Homo sapiens have been studied and their loss of expression is thought to be involved in meningioma production (Peyrard et al. (1994) Hum. Mol. Genet. 3:1393-1399). Beta′ adaptin homologs have been identified in the sequencing projects for Drosophila melanogaster and Arabidopsis thaliana. The cDNAs encoding the 50 kDa subunit from AP2 (AP50) have been isolated from rat brain. Determination of the nucleotide sequence allowed comparison with other known AP50s. This comparison showed that AP50s are highly conserved although there are no significant similarities with other kinases or known proteins (Thurieau et al. (1988) DNA 7:663-669).

Identification of the sequences encoding the different adaptor subunits from a variety of crops may be useful for engineering endocytosis, and stimulating or increasing secretion in plants.

### SUMMARY OF THE INVENTION

Generally, it is the object of the present invention to provide polynucleotides and polypeptides relating to phospholipases. It is an object of the present invention to provide transgenic plants comprising the nucleic acids of the present invention, and methods for modulating, in a transgenic plant, expression of the polynucleotides of the present invention.

The present invention concerns are isolated nucleic acid encoding a polypeptide selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196 and the complement of such sequences.

The present invention concerns an isolated polynucleotide comprising a nucleotide sequence selected from the group consisting of: (a) a first nucleotide sequence encoding a polypeptide of at least 80 amino acids having at least 92% identity based on the Clustal method of alignment when compared to a polypeptide selected from the group consisting of SEQ ID NOs:120, 122, 124, 126, 128, 130, 132, and 134, and (b) a second nucleotide sequence comprising the complement of the first nucleotide sequence.

In a second embodiment, it is preferred that the isolated polynucleotide of the claimed invention comprises a nucleotide sequence which comprises a nucleic acid sequence selected from the group consisting of SEQ ID NOs:119, 121, 123, 125, 127, 129, 131, and 133.

In a third embodiment, this invention concerns an isolated polynucleotide comprising a nucleotide sequence of at least one of 60 (preferably at least one of 40, most preferably at least one of 30) contiguous nucleotides derived from a nucleotide sequence selected from the group consisting of SEQ ID NOs:119, 121, 123, 125, 127, 129, 131, and 133 and the complement of such nucleotide sequences.

In a fourth embodiment, this invention relates to a chimeric gene comprising an isolated polynucleotide of the present invention operably linked to at least one suitable regulatory sequence.

In a fifth embodiment, the present invention concerns a host cell comprising a chimeric gene of the present invention or an isolated polynucleotide of the present invention. The host cell may be eukaryotic, such as a yeast or a plant cell, or prokaryotic, such as a bacterial cell. The present invention also relates to a virus, preferably a baculovirus, comprising an isolated polynucleotide of the present invention or a chimeric gene of the present invention.

In a sixth embodiment, the invention also relates to a process for producing a host cell comprising a chimeric gene of the present invention or an isolated polynucleotide of the present invention, the process comprising either transforming or transfecting a compatible host cell with a chimeric gene or isolated polynucleotide of the present invention.

In a seventh embodiment, the invention concerns a phospholipase D polypeptide of at least 80 amino acids comprising at least 92% identity based on the Clustal method of alignment compared to a polypeptide selected from the group consisting of SEQ ID NOs:120, 122, 124, 126, 128, 130, 132, and 134.

In an eighth embodiment, the invention relates to a method of selecting an isolated polynucleotide that affects the level of expression of a phospholipase D polypeptide or enzyme activity in a host cell, preferably a plant cell, the method comprising the steps of: (a) constructing an isolated polynucleotide of the present invention or a chimeric gene of the present invention; (b) introducing the isolated polynucleotide or the chimeric gene into a host cell; (c) measuring the level of the phospholipase D polypeptide or enzyme activity in the host cell containing the isolated polynucleotide; and (d) comparing the level of the phospholipase D polypeptide or enzyme activity in the host cell containing the isolated polynucleotide with the level of the phospholipase D polypeptide or enzyme activity in the host cell that does not contain the isolated polynucleotide.

In a ninth embodiment, the invention concerns a method of obtaining a nucleic acid fragment encoding a substantial portion of a phospholipase D polypeptide, preferably a plant phospholipase D polypeptide, comprising the steps of: synthesizing an oligonucleotide primer comprising a nucleotide sequence of at least one of 60 (preferably at least one of 40, most preferably at least one of 30) contiguous nucleotides derived from a nucleotide sequence selected from the group consisting of SEQ ID NOs:119, 121, 123, 125, 127, 129, 131, and 133 and the complement of such nucleotide sequences; and amplifying a nucleic acid fragment (preferably a cDNA inserted in a cloning vector) using the oligonucleotide primer. The amplified nucleic acid fragment preferably will encode a substantial portion of a phospholipase D amino acid sequence.

In a tenth embodiment, this invention relates to a method of obtaining a nucleic acid fragment encoding all or a substantial portion of the amino acid sequence encoding a phospholipase D polypeptide comprising the steps of: probing a cDNA or genomic library with an isolated polynucleotide of the present invention; identifying a DNA clone that hybridizes with an isolated polynucleotide of the present invention; isolating the identified DNA clone; and sequencing the cDNA or genomic fragment that comprises the isolated DNA clone.

In an eleventh embodiment, this invention concerns a composition, such as a hybridization mixture, comprising an isolated polynucleotide or polypeptide of the present invention.

In a twelfth embodiment, this invention concerns a method for positive selection of a transformed cell comprising: (a) transforming a host cell with the chimeric gene of the present invention or a construct of the present invention; and (b) growing the transformed host cell, preferably a plant cell, such as a monocot or a dicot, under conditions which allow expression of the phospholipase D polynucleotide in an amount sufficient to complement a null mutant to provide a positive selection means.

In a thirteenth embodiment, this invention relates to a method of altering the level of expression of a phospholipase D in a host cell comprising: (a) transforming a host cell with a chimeric gene of the present invention; and (b) growing the transformed host cell under conditions that are suitable for expression of the chimeric gene wherein expression of the chimeric gene results in production of altered levels of the phospholipase D in the transformed host cell.

### BRIEF DESCRIPTION OF THE SEQUENCE LISTINGS

The invention can be more fully understood from the following detailed description and the accompanying Sequence Listing which form a part of this application.

Table 1 lists the polypeptides that are described herein, the designation of the cDNA clones that comprise the nucleic acid fragments encoding polypeptides representing all or a substantial portion of these polypeptides, and the corresponding identifier (SEQ ID NO:) as used in the attached Sequence Listing. The sequence descriptions and Sequence Listing attached hereto comply with the rules governing nucleotide and/or amino acid sequence disclosures in patent applications as set forth in 37 C.F.R. §1.821-1.825.

Some of the polynucleotide and polypeptide sequences identified in Table 1 are found in previously filed U.S. Provisional Applications as indicated at the bottom of the table.

TABLE 1
Plant Polypeptides

| | | SEQ ID NO: | |
|---|---|---|---|
| Protein | Clone Designation | (Nucleotide) | (Amino Acid) |
| Corn RbokA¹ | p0010.cbpco75rb | 1 | 2 |
| Rice RbohA¹ | rlr6.pk0025.h9 | 3 | 4 |
| Wheat RbohA¹ | wl1n.pk0005.c8 | 5 | 6 |
| Corn RbohA | p0010.cbpco75rb:fis | 7 | 8 |
| Rice RbohA | rlr6.pk0025.h9:fis | 9 | 10 |
| Wheat RbohA | wl1n.pk0005.c8:fis | 11 | 12 |
| Corn RbohB¹ | p0010.cbpaa44rd | 13 | 14 |
| Rice RbohB¹ | rls2.pk0022.d7 | 15 | 16 |
| Soybean RbohB¹ | src2c.pk023.f15 | 17 | 18 |
| Wheat RbohB¹ | wl1n.pk0054.d8 | 19 | 20 |
| Rice RbohB | rls2.pk0022.d7:fis | 21 | 22 |
| Soybean RbohB | src2c.pk023.f15:fis | 23 | 24 |
| Wheat RbohB | wl1n.pk0054.d8:fis | 25 | 26 |
| Rice RbohC² | rlr6.pk0074.e9 | 27 | 28 |
| Rice RbohC | rlr6.pk0074.e9:fis | 29 | 30 |
| Corn RbohD² | Contig of: | 31 | 32 |
| | cco1n.pk055.115 | | |
| | p0127.cntar92r | | |
| Rice RbohD² | rr1.pk0004.a2 | 33 | 34 |
| Soybean RbohD² | sr1.pk0073.f1 | 35 | 36 |
| Wheat RbohD² | wlm96.pk044.g9 | 37 | 38 |
| Rice RbohD | rr1.pk0004.a2:fis | 39 | 40 |
| Soybean RbohD | sr1.pk0073.f1:fis | 41 | 42 |
| Wheat RbohD | wlm96.pk044.g9:fis | 43 | 44 |
| Corn Respiratory Burst | p0104.cabad88rb | 45 | 46 |
| Oxidase Protein³ | | | |
| Rice Respiratory Burst | rsl1n.pk013.i4 | 47 | 48 |
| Oxidase Protein³ | | | |
| Soybean Respiratory Burst | sdp2c.pk009.b13 | 49 | 50 |
| Oxidase Protein³ | | | |
| Corn Respiratory Burst | p0104.cabad88rb:fis | 51 | 52 |
| Oxidase Protein | | | |
| Rice Respiratory Burst | rsl1n.pk013.i4:fis | 53 | 54 |
| Oxidase Protein | | | |
| Soybean Respiratory Burst | sdp2c.pk009.b13:fis | 55 | 56 |
| Oxidase Protein | | | |
| Corn RbohE³ | cen3n.pk0155.f12 | 57 | 58 |
| Soybean RbohE³ | se3.02c07 | 59 | 60 |
| Wheat RbohE³ | wr1.pk178.b5 | 61 | 62 |
| Corn RbohE | cen3n.pk0155.f12:fis | 63 | 64 |
| Wheat RbohE | wrl.pk178.b5:fis | 65 | 66 |
| Corn RbohF³ | p0010.cbpaa44rb | 67 | 68 |
| Soybean RbohF³ | sdp4c.pk014.k19 | 69 | 70 |
| Corn RbohF | p0010.cbpaa44rb:fis | 71 | 72 |
| Soybean RbohF | sdp4c.pk014.k19:fis | 73 | 74 |
| Corn tRNA-mnm⁵s²U-MT⁴ | cco1n.pk077.o18 | 75 | 76 |
| Soybean tRNA-mnm⁵s²U-MT⁴ | se5.pk0029.d2 | 77 | 78 |
| Corn tRNA-mnm⁵s²U-MT | cco1n.pk077.o18:fis | 79 | 80 |
| Soybean tRNA-mnm⁵s²U-MT | se5.pk0029.d2:fis | 81 | 82 |
| Jerusalem Artichoke | hel1.pk0013.b1 | 83 | 84 |
| Chromomethylase⁵ | | | |
| Corn Chromomethylase⁵ | p0094.cssth92ra | 85 | 86 |
| Rice Chromomethylase⁵ | rl0n.pk136.o14 | 87 | 88 |
| Wheat Chromomethylase⁵ | wl1n.pk0095.f3 | 89 | 90 |
| Wheat Chromomethylase⁵ | w1m0.pk0028.h3 | 91 | 92 |
| Jerusalem Artichoke | hel1.pkO0013.b1:fis | 93 | 94 |
| Chromomethylase | | | |
| Corn Chromomethylase | p0094.cssth92ra:fis | 95 | 96 |
| Rice Chromomethylase | rl0n.pk136.o14:fis | 97 | 98 |
| Wheat Chromomethylase | srm.pk0035.c1:fis | 99 | 100 |
| Corn Cytosine | p0100.cbaaj24r | 101 | 102 |
| 5-Methyltransferase⁵ | | | |
| Rice Cytosine | rr1.pk0043.f8 | 103 | 104 |
| 5-Methyltransferase⁵ | | | |
| Soybean Cytosine | sgs2c.pk004.h13 | 105 | 106 |
| 5-Methyltransferase⁵ | | | |
| Wheat Cytosine | wr1.pk0076.a11 | 107 | 108 |
| 5-Methyltransferase⁵ | | | |
| Wheat Cytosine | wre1n.pk0079.c6 | 109 | 110 |
| 5-Methyltransferase⁵ | | | |
| Rice Cytosine | rr1.pk0043.f8:fis | 111 | 112 |
| 5-Methyltransferase | | | |
| Soybean Cytosine | sgs2c.pk004.h13:fis | 113 | 114 |
| 5-Methyltransferase | | | |
| Wheat Cytosine | wr1.pk0076.a11:fis | 115 | 116 |
| 5 -Methyltransferase | | | |
| Wheat Cytosine | wre1n.pk0079.c6:fis | 117 | 118 |
| 5-Methyltransferase | | | |
| Soybean PLD α⁶ | sgs4c.pk004.c18 | 119 | 120 |
| Wheat PLD α⁶ | w1k4.pk0022.b7 | 121 | 122 |
| Soybean PLD α | sfl1.pk128.a18:fis | 123 | 124 |
| Wheat PLD α | wlk4.pk0022.b7:fis | 125 | 126 |
| Corn PLD γ⁶ | p0083.cldaz07r | 127 | 128 |
| Soybean PLD γ⁶ | src3c.pk012.d7 | 129 | 130 |
| Corn PLD γ | p0083.cldaz07r:fis | 131 | 132 |
| Soybean PLD γ | src3c.pk012.d7:fis | 133 | 134 |
| Corn TF IIF α Subunit⁷ | p0026.ccrbd22r | 135 | 136 |
| Corn TF IIF α Subunit | p0026.ccrbd22r:fis | 137 | 138 |
| Corn TF IIF β Subunit⁷ | p0014.ctusq39r | 139 | 140 |
| Wheat TF IIF β Subunit⁷ | wlm24.pk0018.g9 | 141 | 142 |
| Corn TF IIF β Subunit | Contig of: | 143 | 144 |
| | p0014.ctusq39r:fis | | |
| | p0107.cbcap19r | | |
| Rice TF IIF β Subunit | rcaln.pk007.p13:fis | 145 | 146 |
| Rice TF IIF β Subunit | rl0n.pk0063.e10:fis | 147 | 148 |
| Rice TF IIF β Subunit | rls6.pk0059.b8:fis | 149 | 150 |
| Wheat TF IIF β Subunit | wlm24.pk0018.g9:fis | 151 | 152 |
| Corn Asparaginyl-tRNA | p0119.cmtne90r:fis | 153 | 154 |
| Synthetase | | | |
| Rice Asparaginyl-tRNA | rl0n.pk0039.b7:fis | 155 | 156 |
| Synthetase | | | |
| Soybean Asparaginyl-tRNA | src1c.pk001.a5:fis | 157 | 158 |
| Synthetase | | | |
| Wheat Asparaginyl-tRNA | wdr1.pk0005.f7:fis | 159 | 160 |
| synthetase | | | |
| Wheat Asparaginyl-tRNA | wr1.pk0067.h2 | 161 | 162 |
| synthetase | | | |
| Corn Glutaminyl-tRNA | p0129.clmad36r:fis | 163 | 164 |
| synthetase | | | |
| Rice Glutaminyl-tRNA | rds1c.pk007.e9:fis | 165 | 166 |
| synthetase | | | |
| Soybean Glutaminyl-tRNA | sic1c.pk001.e18:fis | 167 | 168 |
| synthetase | | | |
| Wheat Glutaminyl-tRNA | wlmk1.pk0001.g6:fis | 169 | 170 |
| synthetase | | | |
| Rice EDS1 | rl0n.pk127.m10:fis | 171 | 172 |
| Soybean EDS1 | sls2c.pk037.c11:fis | 173 | 174 |
| Wheat EDS1 | wre1n.pk160.d1:fis | 175 | 176 |
| Corn AP50 | p0127.cntam18r | 177 | 178 |
| Rice AP50 | rlr6.pk0083.e10:fis | 179 | 180 |
| Soybean AP50 | sdp3c.pk006.d23:fis | 181 | 182 |
| Wheat AP50 | wdk1c.pk012.n13:fis | 183 | 184 |
| Corn Alpha Adaptin | p0119.cmtoj48r:fis | 185 | 186 |
| Soybean Alpha Adaptin | sl2.pk121.m20:fis | 187 | 188 |
| Corn Beta′ Adaptin | p0119.cmtnr87r:fis | 189 | 190 |
| Rice Beta′ Adaptin | rds1c.pk005.c17:fis | 191 | 192 |
| Soybean Beta′ Adaptin | sls2c.pk005.m4:fis | 193 | 194 |
| Wheat Beta′ Adaptin | wkm2c.pk0002.a3 | 195 | 196 |

¹The polynucleotides listed as SEQ ID NOs:1, 3, 5, 13, 15, 17, and 19 are found as SEQ ID NOs:1, 3, 5, 7, 9, 11, and 13 while the polypeptides listed as SEQ ID NOs:2, 4, 6, 14, 16, 18, and 20 are found as SEQ ID NOs:2, 4, 6, 8, 10, 12, and 14 in U.S. Provisional Application No. 60/143,410, filed Jul. 12, 1999.
²The polynucleotides listed as SEQ ID NOs:27, 31, 33, 35, and 37 are found as SEQ ID NOs:1, 3, 5, 7, and 9 while the polypeptides listed as SEQ ID NOs:28, 32, 34, 36, and 38 are found as SEQ ID NOs:2, 4, 6, 8, and 10 in U.S. Provisional Application No. 60/143,409, filed Jul. 12, 1999.
³The polynucleotides listed as SEQ ID NOs:45, 47, 49, 57, 59, 61, 67, and 69 are found as SEQ ID NOs:1, 3, 5, 7, 9, 11, 13, and 15 while the polypeptides listed as SEQ ID NOs:46, 48, 50, 58, 60, 62, 68, and 70 are found as SEQ ID NOs:2, 4, 6, 8, 10, 12, 14, and 16 in U.S. Provisional Application No. 60/153,534, filed Sep. 13, 1999.
⁴The polynucleotides listed as SEQ ID NOs:77 and 79 and the polypeptides listed as SEQ ID NOs:78 and 80 are found as SEQ ID NOs:1 and 3, and 2 and 4 in U.S. Provisional Application No. 60/143,400, filed Jul. 12, 1999.
⁵The polynucleotides listed as SEQ ID NOs:83, 85, 87, 89, 91, 101, 103, 105, 107, and 109 are found as SEQ ID NOs:1, 3, 5, 7, 9, 11, 13, 15, 17, and 19 while the polypeptides listed as SEQ ID NOs:84, 86, 88, 90, 92, 102, 104, 106, 108, and 110 are found as SEQ ID NOs:2, 4, 6, 8, 10, 12, 14, 16, 18, and 20 in U.S. Provisional Application No. 60/161,223, filed Oct. 22, 1999.
⁶The polynucleotides listed as SEQ ID NOs:119, 121, 127, and 129 are found as SEQ ID NOs:1, 3, 5, and 7 while the polypeptides listed as SEQ ID NOs:120, 122, 128, and 130 are found as SEQ ID NOs:2, 4, 6, and 8 in U.S. Provisional Application No. 60/159,878, filed Oct. 15, 1999.
⁷The polynucleotides listed as SEQ ID NOs:135, 139, and 141 are found as SEQ ID NOs:1, 3, and 5 while the polypeptides listed as SEQ ID NOs:136, 140, and 142 are found as SEQ ID NOs:2, 4, and 6 in U.S. Provisional Application No. 60/157,401, filed Oct. 1, 1999.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

In the context of this disclosure, a number of terms shall be utilized. The terms “polynucleotide”, “polynucleotide sequence”, “nucleic acid sequence”, and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. An isolated polynucleotide of the present invention may include at least one of 60 contiguous nucleotides, preferably at least one of 40 contiguous nucleotides, most preferably one of at least 30 contiguous nucleotides derived from SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, and 195, or the complement of such sequences.

The term “isolated polynucleotide” refers to a polynucleotide that is substantially free from other nucleic acid sequences, such as and not limited to other chromosomal and extrachromosomal DNA and RNA that normally accompany or interact with it as found in its naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The term “recombinant” means, for example, that a nucleic acid sequence is mace by an artificial combination of two otherwise separated segments of sequence, e.g., by chemical synthesis or by the manipulation of isolated nucleic acids by genetic engineering techniques.

As used herein, “contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

As used herein, “substantially similar” refers to nucleic acid fragments wherein changes in one or more nucleotide bases results in substitution of one or more amino acids, but do not affect the functional properties of the polypeptide encoded by the nucleotide sequence. “Substantially similar” also refers to nucleic acid fragments wherein changes in one or more nucleotide bases does not affect the ability of the nucleic acid fragment to mediate alteration of gene expression by gene silencing through for example antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate gene silencing or alteration of the functional properties of the resulting protein molecule. It is therefore understood that the invention encompasses more than the specific exemplary nucleotide or amino acid sequences and includes functional equivalents thereof. The terms “substantially similar” and “corresponding substantially” are used interchangeably herein.

Substantially similar nucleic acid fragments may be selected by screening nucleic acid fragments representing subfragments or modifications of the nucleic acid fragments of the instant invention, wherein one or more nucleotides are substituted, deleted and/or inserted, for their ability to affect the level of the polypeptide encoded by the unmodified nucleic acid fragment in a plant or plant cell. For example, a substantially similar nucleic acid fragment representing at least one of 30 contiguous nucleotides derived from the instant nucleic acid fragment can be constructed and introduced into a plant or plant cell. The level of the polypeptide encoded by the unmodified nucleic acid fragment present in a plant or plant cell exposed to the substantially similar nucleic fragment can then be compared to the level of the polypeptide in a plant or plant cell that is not exposed to the substantially similar nucleic acid fragment.

For example, it is well known in the art that antisense suppression and co-suppression of gene expression may be accomplished using nucleic acid fragments representing less than the entire coding region of a gene, and by using nucleic acid fragments that do not share 100% sequence identity with the gene to be suppressed. Moreover, alterations in a nucleic acid fragment which result in the production of a chemically equivalent amino acid at a given site, but do not effect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. Consequently, an isolated polynucleotide comprising a nucleotide sequence of at least one of 60 (preferably at least one of 40, most preferably at least one of 30) contiguous nucleotides derived from a nucleotide sequence selected from the group consisting of SEQ ID NOs:1, 3, 5, 7, 9, 11, 13, 15, 17, 19, 21, 23, 25, 27, 29, 31, 33, 35. 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, and 195 and the complement of such nucleotide sequences may be used in methods of selecting an isolated polynucleotide that affects the expression of a respiratory burst oxidase homologs, methyltransferases, methylases, phospholipases, transcription factors, aminoacyl-tRNA synthetases, AP-2 subunits, or EDS1 polypeptide in a host cell. A method of selecting an isolated polynucleotide that affects the level of expression of a polypeptide in a virus or in a host cell (eukaryotic, such as plant or yeast, prokaryotic such as bacterial) may comprise the steps of: constructing an isolated polynucleotide of the present invention or a chimeric gene of the present invention; introducing the isolated polynucleotide or the chimeric gene into a host cell; measuring the level of a polypeptide or enzyme activity in the host cell containing the isolated polynucleotide; and comparing the level of a polypeptide or enzyme activity in the host cell containing the isolated polynucleotide with the level of a polypeptide or enzyme activity in a host cell that does not contain the isolated polynucleotide.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions man be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS which was increased to 60° C. Another preferred set of highly stringent conditions uses two final washes in 0.1×SSC, 0.1% SDS at 65° C.

Substantially similar nucleic acid fragments of the instant invention may also be characterized by the percent identity of the amino acid sequences that they encode to the amino acid sequences disclosed herein, as determined by algorithms commonly employed by those skilled in this art. Suitable nucleic acid fragments (isolated polynucleotides of the present invention) encode polypeptides that are at least about 70% identical, preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above identities but typically encode a polypeptide having at least 50 amino acids, preferably at least 100 amino acids, more preferably at least 150 amino acids, still more preferably at least 200 amino acids, and most preferably at least 250 amino acids. Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches it includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215:403-410 (1990); and, Altschul et al., Nucleic Acids Res. 25:3389-3402 (1997).

GAP (Global Alignment Program) can also be used to compare a polynucleotide or polypeptide of the present invention with a reference sequence. GAP uses the algorithm of Needleman and Wunsch (J. Mol. Biol. 48:443-453, 1970) to find the alignment of two complete sequences that maximizes the number of matches and minimizes the number of gaps. GAP considers all possible alignments and gap positions and creates the alignment with the largest number of matched bases and the fewest gaps. The Wisconsin Genetics Software Package for protein sequences uses a gap creation penalty value of 8 and a gap extension penalty value of 2. For polynucleotide sequences, the default gap creation penalty is 50 while the default gap extension penalty is 3. These penalties can be expressed as an integer selected from 0 to 100. Thus, for example, the gap creation and gap extension penalties can each independently be:0, 1, 2, 3, 4, 5, 6, 7, 8, 9, 10, 15, 20, 30, 40, 50, 60 or greater. The scoring matrix used in Version 10 of the Wisconsin Genetics Software Package is BLOSUM62 (see Henikoff & Henikoff (1989) Proc. Natl. Acad. Sci. USA 89:10915).

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually by one skilled in the art, or by using computer-based sequence comparison and identification tools that employ algorithms such as BLAST (Basic Local Alignment Search Tool; Altschul et al. (1993) J. Mol. Biol. 215:403-410; see also www.ncbi.nlm.nih.gov/BLAST/). In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. Accordingly, a “substantial portion” of a nucleotide sequence comprises a nucleotide sequence that will afford specific identification and/or isolation of a nucleic acid fragment comprising the sequence. The instant specification teaches amino acid and nucleotide sequences encoding polypeptides that comprise one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to a nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of the nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign gene” refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a nucleotide sequence that codes for a specific amino acid sequence. “Regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence which can stimulate promoter activity and may be an innate element of the promoter or a heterologous element inserted to enhance the level or tissue-specificity of a promoter. Promoters may be derived in their entirety from a native gene, or may be composed of different elements derived from different promoters found in nature, or may even comprise synthetic nucleotide segments. It is understood by those skilled in the art that different promoters may direct the expression of a gene in different tissues or cell types, or at different stages of development, or in response to different environmental conditions. Promoters which cause a nucleic acid fragment to be expressed in most cell types at most times are commonly referred to as “constitutive promoters”. New promoters of various types useful in plant cells are constantly being discovered; numerous examples may be found in the compilation by Okamuro and Goldberg (1989) Biochemistry of Plants 15:1-82. It is further recognized that since in most cases the exact boundaries of regulatory sequences have not been completely defined, nucleic acid fragments of different lengths may have identical promoter activity.

“Translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Mol. Biotechnol. 3:225-236).

“3′ Non-coding sequences” refers to nucleotide sequences located downstream of a coding sequence and includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNTA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and can be translated into polypeptides by the cell. “cDNA” refers to DNA that is complementary to and derived from an mRNA template. The cDNA can be single-stranded or converted to double stranded form using, for example, the Klenow fragment of DNA polymerase I. “Sense RNA” refers to an RNA transcript that includes the mRNA and can be translated into a polypeptide by the cell. “Antisense RNA” refers to an RNA transcript that is complementary to all or part of a target primary transcript or mRNA and that blocks the expression of a target gene (see U.S. Pat. No. 5,107,065, incorporated herein by reference). The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” refers to the association of two or more nucleic acid fragments so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. “Expression” may also refer to translation of mRNA into a polypeptide. “Anti sense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

A “protein” or “polypeptide” is a chain of amino acids arranged in a specific order determined by the coding sequence in a polynucleotide encoding the polypeptide. Each protein or polypeptide has a unique function.

“Altered levels” or “altered expression” refer to the production of gene product(s) in transgenic organisms in amounts or proportions that differ from that of normal or non-transformed organisms.

“Mature protein” or the term “mature” when used in describing a protein refers to a post-translationally processed polypeptide; i.e., one from which any pre- or propeptides present in the primary translation product have been removed. “Precursor protein” or the term “precursor” when used in describing a protein refers to the primary product of translation of mRNA; i.e., with pre- and propeptides still present. Pre- and propeptides may be but are not limited to intracellular localization signals.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632).

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include 4Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No.4,945,050, incorporated herein by reference). Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Flevin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989 (hereinafter “Maniatis”).

“PCR” or “polymerase chain reaction” is well known by those skilled in the art as a technique used for the amplification of specific DNA segments (U.S. Pat. Nos. 4,683,195 and 4,800,159).

The present invention concerns an isolated polynucleotide comprising a nucleotide sequence selected from the group consisting of: (a) a first nucleotide sequence encoding a polypeptide of at least 80 amino acids having at least 92% identity based on the Clustal method of alignment when compared to a polypeptide selected from the group consisting of SEQ ID NOs:120, 122, 124, 126, 128, 130, 132, and 134, and (b) a second nucleotide sequence comprising the complement of the first nucleotide sequence.

Preferably, the first nucleotide sequence comprises a nucleic acid sequence selected from the group consisting of SEQ ID NOs:119, 121, 123, 125, 127, 129, 131, and 133, that codes for the polypeptide selected from the group consisting of SEQ ID NOs:120, 122, 124, 126, 128, 130, 132, and 134.

Nucleic acid fragments encoding at least a substantial portion of several plant polypeptides have been isolated and identified by comparison of random plant cDNA sequences to public databases containing nucleotide and protein sequences using the BLAST algorithms well known to those skilled in the art. The nucleic acid fragments of the instant invention may be used to isolate cDNAs and genes encoding homologous proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other respiratory burst oxidase homologs, methyltransferases, methylases, phospholipases, transcription factors, aminoacyl-tRNA synthetases, AP-2 subunits, or EDS1, either as cDNAs or genomic DNAs, could be isolated directly by using all or a substantial portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, entire sequence(s) can be used directly to synthesize DNA probes by methods known to the skilled artisan such as random primer DNA labeling, nick translation, end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al. (1988) Proc. Natl. Acad. Sci. USA 85:8998-9002) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al. (1989) Proc. Natl. Acad Sci. USA 86:5673-5677; Loh et al. (1989) Science 243:217-220). Products generated by the 3′ and 5′ RACE procedures can be combined to generate full-length cDNAs (Frohman and Martin (1989) Techniques 1:165). Consequently, a polynucleotide comprising a nucleotide sequence of at least one of 60 (preferably one of at least 40, most preferably one of at least 30) contiguous nucleotides derived from a nucleotide sequence selected from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, and 195 and the complement of such nucleotide sequences may be used in such methods to obtain a nucleic acid fragment encoding a substantial portion of an amino acid sequence of a polypeptide.

The present invention relates to a method of obtaining a nucleic acid fragment encoding a substantial portion of a respiratory burst oxidase homolog, methyltransferase, methylase, phospholipase, transcription factor, aminoacyl-tRNA synthetase, AP-2 subunit, or EDS1 polypeptide, preferably a substantial portion of a plant respiratory burst oxidase homolog, methyltransferase, methylase, phospholipase, transcription factor, aminoacyl-tRNA synthetase, AP-2 subunit, or EDS1 polypeptide, comprising the steps of: synthesizing an oligonucleotide primer comprising a nucleotide sequence of at least one of 60 (preferably at least one of 40, most preferably at least one of 30) contiguous nucleotides derived from a nucleotide sequence selected from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, and 195, and the complement of such nucleotide sequences; and amplifying a nucleic acid fragment (preferably a cDNA inserted in a cloning vector) using the oligonucleotide primer. The amplified nucleic acid fragment preferably will encode a substantial portion of a respiratory burst oxidase homolog, methyltransferase, methylase, phospholipase, transcription factor, aminoacyl-tRNA synthetase, AP-2 subunit, or EDS1 polypeptide.

Availability of the instant nucleotide and deduced amino acid sequences facilitates immunological screening of cDNA expression libraries. Synthetic peptides representing substantial portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner (1984) Adv. Immunol. 36:1-34; Maniatis).

In another embodiment, this invention concerns viruses and host cells comprising either the chimeric genes of the invention as described herein or an isolated polynucleotide of the invention as described herein. Examples of host cells which can be used to practice the invention include, but are not limited to, yeast, bacteria, and plants.

As was noted above, the nucleic acid fragments of the instant invention may be used to create transgenic plants in which the disclosed polypeptides are present at higher or lower levels than normal or in cell types or developmental stages in which they are not normally found. This would have the effect of altering the level of stress and disease resistance, enhancement of gene expression or transcription, quality grain improvement, or generation of novel starches in those cells.

Overexpression of the proteins of the instant invention may be accomplished by first constructing a chimeric gene in which the coding region is operably linked to a promoter capable of directing expression of a gene in the desired tissues at the desired stage of development. The chimeric gene may comprise promoter sequences and translation leader sequences derived from the same genes. 3′ Non-coding sequences encoding transcription termination signals may also be provided. The instant chimeric gene may also comprise one or more introns in order to facilitate gene expression.

Plasmid vectors comprising the instant isolated polynucleotide (or chimeric gene) may be constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the instant polypeptides to different cellular compartments, or to facilitate their secretion from the cell. It is thus envisioned that the chimeric gene described above may be further supplemented by directing the coding sequence to encode the instant polypeptides with appropriate intracellular targeting sequences such as transit sequences (Keegstra (1989) Cell 56:247-253), signal sequences or sequences encoding endoplasmic reticulum localization (Chrispeels (1991) Ann. Rev. Plant Phys. Plant Mol Biol. 42:21-53), or nuclear localization signals (Raikhel (1992) Plant Phys. 100:1627-1632) with or without removing targeting sequences that are already present. While the references cited give examples of each of these, the list is not exhaustive and more targeting signals of use may be discovered in the future.

It may also be desirable to reduce or eliminate expression of genes encoding the instant polypeptides in plants for some applications. In order to accomplish this, a chimeric gene designed for co-suppression of the instant polypeptide can be constructed by linking a gene or gene fragment encoding that polypeptide to plant promoter sequences. Alternatively, a chimeric gene designed to express antisense RNA for all or part of the instant nucleic acid fragment can be constructed by linking the gene or gene fragment in reverse orientation to plant promoter sequences. Either the co-suppression or antisense chimeric genes could be introduced into plants via transformation wherein expression of the corresponding endogenous genes are reduced or eliminated.

Molecular genetic solutions to the generation of plants with altered gene expression have a decided advantage over more traditional plant breeding approaches. Changes in plant phenotypes can be produced by specifically inhibiting expression of one or more genes by antisense inhibition or co-suppression (U.S. Pat. Nos. 5,190,931, 5,107,065 and 5,283,323). An antisense or co-suppression construct would act as a dominant negative regulator of gene activity. While conventional mutations can yield negative regulation of gene activity these effects are most likely recessive. The dominant negative regulation available with a transgenic approach may be advantageous from a breeding perspective. In addition, the ability to restrict the expression of a specific phenotype to the reproductive tissues of the plant by the use of tissue specific promoters may confer agronomic advantages relative to conventional mutations which may have an effect in all tissues in which a mutant gene is ordinarily expressed.

The person skilled in the art will know that special considerations are associated with the use of antisense or cosuppression technologies in order to reduce expression of particular genes. For example, the proper level of expression of sense or antisense genes may require the use of different chimeric genes utilizing different regulatory elements known to the skilled artisan. Once transgenic plants are obtained by one of the methods described above, it will be necessary to screen individual transgenics for those that most effectively display the desired phenotype. Accordingly, the skilled artisan will develop methods for screening large numbers of transformants. The nature of these screens will generally be chosen on practical grounds. For example, one can screen by looking for changes in gene expression by using antibodies specific for the protein encoded by the gene being suppressed, or one could establish assays that specifically measure enzyme activity. A preferred method will be one which allows large numbers of samples to be processed rapidly, since it will be expected that a large number of transformants will be negative for the desired phenotype.

In another embodiment, the present invention concerns a polypeptide of at least 80 amino acids having at least 92% identity based on the Clustal method of alignment when compared to a polypeptide selected from the group consisting of SEQ ID NOs:120, 122, 124, 126, 128, 130, 132 and 134.

The instant polypeptides (or substantial portions thereof) may be produced in heterologous host cells, particularly in the cells of microbial hosts, and can be used to prepare antibodies to these proteins by methods well known to those skilled in the art. The antibodies are useful for detecting the polypeptides of the instant invention in situ in cells or in vitro in cell extracts. Preferred heterologous host cells for production of the instant polypeptides are microbial hosts. Microbial expression systems and expression vectors containing regulatory sequences that direct high level expression of foreign proteins are well known to those skilled in the art. Any of these could be used to construct a chimeric gene for production of the instant polypeptides. This chimeric gene could then be introduced into appropriate microorganisms via transformation to provide high level expression of the encoded polypeptide. An example of a vector for high level expression of the instant polypeptides in a bacterial host is provided (Example 25).

Additionally, some of the instant polypeptides can be used as a target to facilitate design and/or identification of inhibitors of those enzymes that may be useful as herbicides. This is desirable because the polypeptides described herein catalyze various steps in RNA processing. Accordingly, inhibition of the activity of one or more of the enzymes described herein could lead to inhibition of plant growth. Thus, the instant polypeptides could be appropriate for new herbicide discovery and design.

All or a substantial portion of the polynucleotides of the instant invention may also be used as probes for genetically and physically mapping the genes that they are a part of, and used as markers for traits linked to those genes. Such information may be useful in plant breeding in order to develop lines with desired phenotypes. For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et al. (1987) Genomics 1:174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4:37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel et al. In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask (1991) Trends Genet. 7:149-154). Although current methods of FISH mapping favor use of large clones (several to several hundred KB; see Laan et al. (1995) Genome Res. 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer (1989) Proc. Natl. Acad. Sci USA 86:9402-9406; Koes et al. (1995) Proc. Natl. Acad. Sci USA 92:8149-8153; Bensen et al. (1995) Plant Cell 7:75-84). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the instant polypeptides. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding the instant polypeptides can be identified and obtained. This mutant plant can then be used to determine or confirm the natural function of the instant polypeptides disclosed herein.

The present invention provides machines, articles of manufacture, and processes for identifying, modeling, or analyzing the polynucleotides and polypeptides of the present invention. Identification methods permit identification of homologues of the polynucleotides or polypeptides of the present invention, while modeling and analysis methods permit recognition of structural or functional features of interest.

In one embodiment, the present invention provides a machine having: 1) a memory comprising data representing at least one genetic sequence, 2) a genetic identification, analysis, or modeling program with access to the data, 3) a data processor which executes instructions according to the program using the genetic sequence or a subsequence thereof, and 4) an output for storing or displaying the results of the data processing.

The machine of the present invention is a data processing system, typically a digital computer. The term “computer” includes one or several desktop or portable computers, computer workstations, servers (including intranet or internet servers), mainframes, and any integrated system comprising any of the above irrespective of whether the processing, memory, input, or output of the computer is remote or local, as well as any network interconnecting the modules of the computer. Data processing can thus be remote or distributed amongst several processors at a single or multiple sites. The data processing system comprises a data processor, such as a central processing unit (CPU), which executes instructions according to an application program. As used herein, machines, articles of manufacture, and processes are exclusive of the machines, manufactures, and processes employed by the United States Patent and Trademark Office or the European Patent Office for patentability searches using data representing the sequence of a polypeptide or polynucleotide of the present invention.

The machine of the present invention further includes a memory, comprising data representing at least one genetic sequence. As used herein, “genetic sequence” refers to the primary sequence (i.e., amino acid or nucleotide sequence) of a polynucleotide or polypeptide of the present invention. The genetic sequence can represent a partial sequence from a full-length protein, genomic DNA, or full-length cDNA/mRNA. Nucleic acids or proteins comprising a genetic sequence that is identified, analyzed, or modeled according to the present invention can be cloned or synthesized.

As those of skill in the art will be aware, the form of memory of a machine of the present invention, or the particular embodiment of the computer readable medium, are not critical elements of the invention and can take a variety of forms. The memory of such a machine includes, but is not limited to, ROM, RAM, or computer readable media such as, but not limited to, magnetic media such as computer disks or hard drives, or media such as CD-ROMs, DVDs, and the like. The memory comprising the data representing the genetic sequence includes main memory, a register, and a cache. In some embodiments the data processing system stores the data representing the genetic sequence in memory while processing the data and wherein successive portions of the data are copied sequentially into at least one register of the data processor for processing. Thus, the genetic sequence stored in memory can be a genetic sequence created during computer runtime or stored beforehand. The machine of the present invention includes a genetic identification, analysis, or modeling program (discussed below) with access to the data representing the genetic sequence. The program can be implemented in software or hardware.

The present invention further contemplates that the machine of the present invention will reference, directly or indirectly, a utility or function for the polynucleotide or polypeptide of the present invention. For example, the utility/function can be directly referenced as a data element in the machine and accessible by the program. Alternatively, the utility/function of the genetic can be indirectly referenced to an electronic or written record. The function or utility of the genetic sequence can be a function or utility for the genetic sequence, or the data representing the sequence (i.e., the genetic sequence data). Exemplary function or utilities for the genetic sequence include: 1) its name (per International Union of Biochemistry and Molecular Biology rules of nomenclature) or the function of the enzyme or protein represented by the genetic sequence, 2) the metabolic pathway that the protein represented by the genetic sequence participates in, 3) the substrate, product or structural role of the protein represented by the genetic sequence, or, 4) the phenotype (e.g., an agronomic or pharmacological trait) affected by modulating expression or activity of the protein represented by the genetic sequence.

The machine of the present invention also includes an output for displaying, printing, or recording the results of the identification, analysis, or modeling performed using a genetic sequence of the present invention. Exemplary outputs include monitors, printers, or various electronic storage mechanisms (e.g., floppy disks, hard drives, main memory) which can be used to display the results or employed as a means to input the stored data into a subsequent application or device.

In some embodiments, data representing a genetic sequence of the present invention is a data element within a data structure. The data structure may be defined by the computer programs that define the processes of identification, modeling, or analysis (see below) or it may be defined by the programming of separate data storage and retrieval programs, subroutines or systems. Thus, the present invention provides a memory for storing a data structure that can be accessed by a computer programmed to implement a process for identification, analysis, or modeling of a genetic sequence. The data structure, stored within memory, is associated with the data representing the genetic sequence and reflects the underlying organization and structure of the genetic sequence to facilitate program access to data elements corresponding to logical sub-components of the genetic sequence. The data structure enables the genetic sequence to be identified, analyzed, or modeled. The underlying order and structure of a genetic sequence is data representing the higher order organization of the primary sequence. Such higher order structures affect transcription, translation, enzyme kinetics, or reflects structural domains or motifs. Exemplary logical sub-components which constitute the higher order organization of the genetic sequence include but are not limited to: restriction enzyme sites, endopeptidase sites, major grooves, minor grooves, beta-sheets, alpha helices, open reading frames (ORFs), 5′ untranslated regions (UTRs), 3′ UTRs, ribosome binding sites, glycosylation sites, signal peptide domains, intron-exon junctions, poly-A tails, transcription initiation sites, translation start sites, translation termination sites, methylation sites, zinc finger domains, modified amino acid sites, preproprotein-proprotein junctions, proprotein-protein junctions, transit peptide domains, single nucleotide polymorphisms (SNPs), simple sequence repeats (SSRs), restriction fragment length polymorphisms (RFLPs), insertion elements, transmembrane spanning regions, and stem-loop structures.

In another embodiment, the present invention provides a data processing system comprising at least one data structure in memory where the data structure supports the accession of data representing a genetic sequence of the present invention. The system also comprises at least one genetic identification, analysis, or modeling program which directs the execution of instructions by the system using the genetic sequence data to identify, analyze, or model at least one data element which is a logical sub-component of the genetic sequence. An output for the processing results is also provided.

In another embodiment, the present invention provides a data structure in a computer readable medium that contains data representing a genetic sequence of the present invention. The data structure is organized to reflect the logical structuring of the genetic sequence, so that the sequence can be analyzed by software programs capable of accessing the data structure. In particular, the data structures of the present invention organize the genetic sequences of the present invention in a manner which allows software tools to perform an identification, analysis, or modeling using logical elements of each genetic sequence.

In a further embodiment, the present invention provides a machine-readable media containing a computer program and genetic sequence data. The program provides instructions sufficient to implement a process for effecting the identification, analysis, or modeling of the genetic sequence data. The media also includes a data structure reflecting the underlying organization and structure of the data to facilitate program access to data elements corresponding to logical sub-components of the genetic sequence, the data structure being inherent in the program and in the way in which the program organizes and accesses the data.

An example of a data structure resembles a layered hash table, where in one dimension the base content of the sequence is represented by a string of elements A, T, C, G and N. The direction from the 5′ end to the 3′ end is reflected by the order from the position 0 to the position of the length of the string minus one. Such a string, corresponding to a nucleotide sequence of interest, has a certain number of substrings, each of which is delimited by the string position of its 5′ end and the string position of its 3′ end within the parent string. In a second dimension, each substring is associated with or pointed to one or multiple attribute fields. Such attribute fields contain annotations to the region on the nucleotide sequence represented by the substring.

For example, a sequence under investigation is 520 bases long and represented by a string named SeqTarget. There is a minor groove in the 5′ upstream non-coding region from position 12 to 38, which is identified as a binding site for an enhancer protein HM-A, which in turn will increase the transcription of the gene represented by SeqTarget. Here, the substring is represented as (12, 38) and has the following attributes: [upstream uncoded], [minor groove], [HM-A binding] and [increase transcription upon binding by HM-A]. Similarly, other types of information can be stored and structured in this manner, such as information related to the whole sequence, e.g., whether the sequence is a full length viral gene, a mammalian house keeping gene, an EST from clone X, or information related to the 3′ down stream non-coding region, e.g., hairpin structure, and information related to various domains of the coding region, e.g., Zinc finger.

This data structure is an open structure and is robust enough to accommodate newly generated data and acquired knowledge. Such a structure is also a flexible structure. It can be trimmed down to a 1-D string to facilitate data mining and analysis steps, such as clustering, repeat-masking, and HMM analysis. Meanwhile, such a data structure also can extend the associated attributes into multiple dimensions. Pointers can be established among the dimensioned attributes when needed to facilitate data management and processing in a comprehensive genomics knowledgebase. Furthermore, such a data structure is object-oriented. Polymorphism can be represented by a family or class of sequence objects, each of which has an internal structure as discussed above. The common traits are abstracted and assigned to the parent object, whereas each child object represents a specific variant of the family or class. Such a data structure allows data to be efficiently retrieved, updated and integrated by the software applications associated with the sequence database and/or knowledgebase.

The present invention also provides a process of identifying, analyzing, or modeling data representing a genetic sequence of the present invention. The process comprises: 1) providing a machine having a hardware or software implemented genetic sequence identification, modeling, or analysis program with data representing a genetic sequence, 2) executing the program while granting it access to the genetic sequence data, and 3) displaying or outputting the results of the identification, analysis, or modeling. Data structures made by the processes of the present invention and embodied within a computer readable medium are also provided herein.

A further process of the present invention comprises providing a memory embodied with data representing a genetic sequence and developing within the memory a data structure associated with the data and reflecting the underlying organization and structure of the data to facilitate program access to data elements corresponding to logical sub-components of the sequence. A computer is programmed with a program containing instructions sufficient to implement the process for effecting the identification, analysis, or modeling of the genetic sequence and the program is executed on the computer while granting the program access to the data and to the data structure within the memory. The program results are outputted.

Identification, analysis, and modeling programs are well known in the art and available commercially. The program typically has at least one application to: 1) identify the structural role or enzymatic function of the gene which the genetic sequence encodes or is translated from, 2) analyzes and identifies higher order structures within the genetic sequence or, 3) model the physico-chemical properties of a genetic sequence of the present invention in a particular environment.

Included amongst the modeling/analysis tools are methods to: 1) recognize overlapping sequences (e.g., from a sequencing project) with a polynucleotide of the present invention and create an alignment called a “contig” ; 2) identify restriction enzyme sites of a polynucleotide of the present invention; 3) identify the products of a T1 ribonuclease digestion of a polynucleotide of the present invention; 4) identify PCR primers with minimal self-complementarity; 5) compute pairwise distances between sequences in an alignment, reconstruct phylogentic trees using distance methods, and calculate the degree of divergence of two protein coding regions; 6) identify patterns such as coding regions, terminators, repeats, and other consensus patterns in polynucleotides of the present invention; 7) identify RNA secondary structure; 8) identify sequence motifs, isoelectric point, secondary structure, hydrophobicity, and antigenicity in polypeptides of the present invention; 9) translate polynucleotides of the present invention and backtranslate polypeptides of the present invention; and 10) compare two protein or nucleic acid sequences and identifying points of similarity or dissimilarity between them.

Identification of the function/utility of a genetic sequence is typically achieved by comparative analysis to a gene/protein database and establishing the genetic sequence as a candidate homologue (i.e., ortholog or paralog) of a gene/protein of known function/utility. A candidate homologue has statistically significant probability of having the same biological function (e.g., catalyzes the same reaction, binds to homologous proteins/nucleic acids, has a similar structural role) as the reference sequence to which it is compared. Sequence identity/similarity is frequently employed as a criterion to identify candidate homologues. In the same vein, genetic sequences of the present invention have utility in identifying homologs in animals or other plant species, particularly those in the family Gramineae such as, but not limited to, sorghum, wheat, or rice. Function is frequently established on the basis of sequence identity/similarity.

Exemplary sequence comparison systems are provided for in sequence analysis software such as those provided by the Genetics Computer Group (Madison, Wis.) or InforMax (Bethesda, Md.), or Intelligenetics (Mountain View, Calif.). Optionally, sequence comparison is established using the BLAST or GAP suite of programs. Generally, a smallest sum probability value (P(N)) of less than 0.1, or alternatively, less than 0.01, 0.001, 0.0001, or 0.00001 using the BLAST 2.0 suite of algorithms under default parameters identifies the test sequence as a candidate homologue (i.e., an allele, ortholog, or paralog) of a reference sequence. Those of skill in the art will recognize that a candidate homologue has an increased statistical probability of having the same or similar function as the gene/protein represented by the test sequence.

The software/hardware for effecting identification, analysis, or modeling can be produced independently or obtained from commercial suppliers. Exemplary identification, analysis, and modeling tools are provided in products such as InforMax's (Bethesda, Md.) Vector NTI Suite (Version 5.5), Intelligenetics' (Mountain View, Calif.) PC/Gene program, and Genetics Computer Group's (Madison, Wis.) Wisconsin Package (Version 10.0); these tools, and the functions they perform, (as provided and disclosed by the programs and accompanying literature) are incorporated herein by reference.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only and are not to limit the scope of the invention. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of all publications, patents, patent applications, and computer programs cited herein are hereby incorporated by reference in their entirety.

### Example 1

### Composition of cDNA Libraries; Isolation and Sequencing of cDNA Clones

cDNA libraries representing mRNAs from various corn, Jerusalem artichoke, rice, soybean, and wheat tissues were prepared. The characteristics of the libraries are described below.

TABLE 2
cDNA Libraries from Corn, Jerusalem Artichoke, Rice, Soybean, and Wheat

| Library | Tissue | Clone |
|---|---|---|
| cco1n | Corn Cob of 67 Day Old Plants Grown in Green House¹ | cco1n.pk055.115 |
| | | cco1n.pk077.o18 |
| cen3n | Corn Endosperm 20 Days After Pollination¹ | cen3n.pk0155.f12 |
| he11 | Jerusalem Artichoke Tuber at Filling Stage | he11.pk0013.b1 |
| p0010 | Corn Log Phase Suspension Cells Treated With | p0010.cbpaa44rb |
| | A23187² to Induce Mass Apoptosis | p0010.cbpaa44rd |
| | | p0010.cbpco75rb |
| p0014 | Corn Leaves 7 and 8 from Plant Transformed With | p0014.ctusq39r |
| | G-protein Gene, C. heterostrophus Resistant | |
| p0026 | Corn Regenerating Callus 5 Days After Auxin Removal | p0026.ccrbd22r |
| p0083 | Corn Whole Kernels 7 Days After Pollination | p0083.c1daz07r |
| p0094 | Corn Leaf Collars for the Ear Leaf (EL) and the | p0094.cssth92ra |
| | Next Leaf Above and Below the EL¹ | |
| p0100 | Corn Coenocytic Embryo Sacs 4 Days After Pollination¹ | p0100.cbaaj24r |
| p0104 | Corn Roots Stage V5³, Infested With Corn Root Worm¹ | p0104.cabad88rb |
| p0107 | Corn Whole Kernels 7 Days After Pollination¹ | p0107.cbcap19r |
| p0119 | Corn V12-Stage^{3 Ear Shoot With Husk, Night Harvested}¹ | p0119.cmtne90r |
| | | p0119.cmtnr87r:fis |
| | | p0119.cmtoj48r:fis |
| p0127 | Corn Nucellus Tissue, 5 Days After Silking¹ | p0127.cntam18r |
| | | p0127.cntar92r |
| p0129 | H08 Lazy Mutant Internode Tissue | p0129.c1mad36r:fis |
| rca1n | Rice Callus¹ | rca1n.pk007.p13:fis |
| rds1c | Rice Developing Seeds | rds1c.pk005.c17:fis |
| | | rds1c.pk007.e9:fis |
| r10n | Rice 15 Day Old Leaf¹ | r10n.pk0039.b7:fis |
| | | r10n.pk0063.e10 |
| | | r10n.pk127.m10:fis |
| | | r10n.pk136.o14 |
| r1r6 | Rice Leaf 15 Days After Germination, 6 Hours After | r1r6.pk0025.h9 |
| | Infection of Strain Magaporthe grisea 4360-R-62 | r1r6.pk0074.e9 |
| | (AVR2-YAMO); Resistant | r1r6.pk0083.e10:fis |
| r1s2 | Rice Leaf 15 Days After Germination, 2 Hours After | r1s2.pk0022.d7 |
| | Infection of Strain Magaporthe grisea 4360-R-67 | |
| | (AVR2-YAMO); Susceptible | |
| r1s6 | Rice Leaf 15 Days After Germination, 6 Hours After | r1s6.pk0059.b8 |
| | Infection of Strain Magaporthe grisea 4360-R-67 | |
| | (AVR2-YAMO); Susceptible | |
| rr1 | Rice Root of Two Week Old Developing Seedling | rr1.pk0004.a2 |
| | | rr1.pk0043.f8 |
| rs11n | Rice 15-Day-Old Seedling¹ | rs11n.pk013.14 |
| sdp2c | Soybean Developing Pods (6-7 mm) | sdp2c.pk009.b13 |
| sdp3c | Soybean Developing Pods (8-9 mm) | sdp3c.pk006.d23:fis |
| sdp4c | Soybean Developing Pods (10-12 mm) | sdp4c.pk014.k19 |
| se3 | Soybean Embryo, 17 Days After Flowering | se3.02c07 |
| se5 | Soybean Embryo, 21 Days After Flowering | se5.pk0029.d2 |
| sf11 | Soybean Immature Flower | sf11.pk128.a18:fis |
| sgc2c | Soybean Cotyledon 12-20 Days After Germination | sgs2c.pk004.h13 |
| | (Mature Green) | |
| sgc4c | Soybean Cotyledon 14-21 Days After Germination | sgs4c.pk004.c18 |
| | (1/4 yellow) | |
| sic1c | Soybean Root, Stem, and Leaf Tissue With Iron | sic1c.pk001.e18:fis |
| | Chlorosis, Pooled | |
| s12 | Soybean Two-Week-Old Developing Seedlings | s12.pk121.m20:fis |
| | Treated With 2.5 ppm chlorimuron | |
| s1s2c | Soybean Infected With Scierotinia scierotiorum | s1s2c.pk005.m4:fis |
| | Mycelium | s1s2c.pk037.c11 |
| sr1 | Soybean Root | sr1.pk0073.f1 |
| src1c | Soybean 8 Day Old Root Infected With Cyst Nematode | src1c.pk001.a5:fis |
| src2c | Soybean 8 Day Old Root Infected With Cyst Nematode | src2c.pk023.f15 |
| src3c | Soybean 8 Day Old Root Infected With Cyst Nematode | src3c.pk012.d7 |
| srm | Soybean Root Meristem | srm.pk0035.c1:fis |
| wdk1c | Wheat Developing Kernel, 3 Days After Anthesis | wdk1c.pk012.n13:fis |
| wdr1 | Wheat Developing Root and Leaf | wdr1.pk0005.f7:fis |
| wkm2c | Wheat Kernel Malted 175 Hours at 4 Degrees Celsius | wkm2c.pk0002.a3 |
| w11n | Wheat Leaf From 7 Day Old Seedling¹ | w11n.pk0005.c8 |
| | | w11n.pk0054.d8 |
| | | w11n.pk0095.f3:fis |
| w1k4 | Wheat Seedlings 4 Hours After Treatment With Herbicide⁴ | w1k4.pk0022.b7 |
| w1m0 | Wheat Seedlings 0 Hour After Inoculation With | w1m0.pk0028.h3:fis |
| | Erysiphe graminis f.sp tritici | |
| w1m24 | Wheat Seedlings 24 Hours After Inoculation With | w1m24.pk0018.g9 |
| | Erysiphe graminis f.sp tritici | |
| w1m96 | Wheat Seedlings 96 Hours After Inoculation With | w1m96.pk044.g9 |
| | Erysiphe graminis f.sp tritici | |
| w1mk1 | Wheat Seedlings 1 Hour After Inoculation With | w1mk1.pk0001.g6:fis |
| | Erysiphe graminis f.sp tritici and Treatment With | |
| | Herbicide⁴ | |
| wr1 | Wheat Root From 7 Day Old Seedling | wr1.pk0067.h2 |
| | | wr1.pk0076.a11 |
| | | wr1.pk178.b5 |
| wre1n | Wheat Root From 7 Day Old Etiolated Seedling¹ | wre1n.pk0079.c6 |
| | | wre1n.pk160.d1:fis |

¹These libraries were normalized essentially as described in U.S. Pat. No. 5,482,845, incorporated herein by reference.
²A23187 is commercially available from several vendors including Calbiochem.
³Corn developmental stages are explained in the publication “How a corn plant develops” from the Iowa State University Coop. Ext. Service Special Report No. 48 reprinted June 1993.
⁴Application of 6-iodo-2-propoxy-3-propyl-4(3H)-quinazolinone; synthesis and methods of using this compound are described in U.S. Pat. No. 5,747,497, incorporated herein by reference.

cDNA libraries may be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts is will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones are randomly selected from each transposition reaction, plasmid DNAs are prepared via alkaline lysis, and templates are sequenced (ABI Prism dye-terminator ReadyReaction mix) outward from the transposition event site, utilizing unique primers specific to the binding sites within the transposon.

Sequence data is collected (ABI Prism Collections) and assembled using Phred/Phrap (P. Green, University of Washington, Seattle). Phrep/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (D. Gordon, University of Washington, Seattle).

### Example 2

### Identification of cDNA Clones

cDNA clones encoding respiratory burst oxidase homologs, methyltransferases, methylases, phospholipases, transcription factors, aminoacyl-tRNA synthetases, AP2 subunits, or EDS1 were identified by conducting BLAST (Basic Local Alignment Search Tool; Altschul et al. (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

ESTs submitted for analysis are compared to the genbank database as described above. ESTs that contain sequences more 5-prime or 3-prime can be found by using the BLASTN algorithm (Altschul et al (1997) Nucleic Acids Res. 25:3389-3402.) against the Du Pont proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5-prime or 3-prime direction. Once the most 5-prime EST is identified, its complete sequence can be determined by Full Insert Sequencing as described in Example 1. Homologous genes belonging to different species can be found by comparing the amino acid sequence of a known gene (from either a proprietary source or a public database) against an EST database using the TBLASTN algorithm. The TBLASTN algorithm searches an amino acid query against a nucleotide database that is translated in all 6 reading frames. This search allows for differences in nucleotide codon usage between different species, and for codon degeneracy.

### Example 3

### Characterization of cDNA Clones Encoding RbohA

The BLASTX search using the EST sequences from clones listed in Table 3 revealed similarity of the polypeptides encoded by the Contig to respiratory burst oxidase homolog A (RbohA) from Arabidopsis thaliana (NCBI General Identifier No. 3242781). Shown in Table 3 are the BLAST results for individual ESTs (“EST”):

TABLE 3
BLAST Results for Sequences Encoding
Polypeptides Homologous to RbohA

| | | BLAST pLog Score |
|---|---|---|
| Clone | Status | 3242781 (Arabidopsis thaliana) |
| p0010.cbpco75rb | EST | 46.40 |
| r1r6.pk0025.h9 | EST | 69.00 |
| w11n.pk0005.c8 | EST | 53.00 |

The sequence of the entire cDNA insert in the clones listed in Table 3 was determined. The BLASTX search using the EST sequences from clones listed in Table 4 revealed similarity of the polypeptides encoded by the Contig to RbohA from Arabidopsis thaliana (NCBI General Identifier No. 3242781) and by the by the Contig to RbohB from Arabidopsis thaliana (NCBI General Identifier No. 3242783). Shown in Table 4 are the BLAST results for the sequences of the entire cDNA inserts comprising the indicated cDNA clones (“FIS”):

TABLE 4
BLAST Results for Sequences Encoding Polypeptides Homologous
to Arabidopsis thaliana RbohA and RbohB

| | | BLAST pLog Score | |
|---|---|---|---|
| Clone | Status | 3242781 (RbohA) | 3242783 (RbohB) |
| p0010.cbpco75rb:fis | FIS | 56.40 | 60.52 |
| r1r6.pk0025.h9:fis | FIS | 63.00 | 59.70 |
| w11n.pk0005.c8:fis | FIS | 54.22 | 51.70 |

The data in Table 5 presents a calculation of the percent identity of the amino acid sequences set forth in SEQ ID NOs:2, 4, 6, 8, 10, and 12 and the Arabidopsis thaliana RbohA and RbohB sequences (NCBI General Identifier Nos. 3242781 and 3242783, respectively).

TABLE 5
Percent Identity of Amino Acid Sequences Deduced From the Nucleotide
Sequences of cDNA Clones Encoding Polypeptides Homologous
to Arabidopsis thaliana RbohA and RbohB

Percent Identity to

| SEQ ID NO. | 3242781 (RbohA) | 3242783 (RbohB) |
|---|---|---|
| 2 | 57.5 | 55.2 |
| 4 | 83.6 | 75.0 |
| 6 | 79.5 | 73.0 |
| 8 | 60.0 | 62.4 |
| 10 | 82.5 | 76.6 |
| 12 | 80.6 | 75.8 |

Sequence alignments and percent identity calculations were performed using the Megalign program of the LASERGENE bioinformatics computing suite (DNASTAR Inc., Madison, Wis.) Multiple alignment of the sequences was performed using the Clustal method of alignment (Higgins and Sharp (1989) CABIOS. 5:151-153) with the default parameters(GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, and BLAST scores and probabilities indicate that the nucleic acid fragments comprising the instant cDNA clones encode substantial portions of a corn, a rice, and a wheat respiratory burst oxidase homolog.

### Example 4

### Characterization of cDNA Clones Encoding RbohB

The BLASTX search using the EST sequences from clones listed in Table 6 revealed similarity of the polypeptides encoded by the cDNAs to respiratory burst oxidase homolog B (RbohB) from Arabidopsis thaliana (NCBI General Identifier No. 3242783). Shown in Table 6 are the BLAST results for individual ESTs (“EST”):

TABLE 6
BLAST Results for Sequences Encoding
Polypeptides Homologous to RbohB

| | | BLAST pLog Score |
|---|---|---|
| Clone | Status | 3242783 (Arabidopsis thaliana) |
| p0010.cbpaa44rd | EST | 86.00 |
| r1s2.pk0022.d7 | EST | 35.40 |
| src2c.pk023.f15 | EST | 52.70 |
| w11n.pk0054.d8 | EST | 35.00 |

The sequence of the entire cDNA insert in the rice, soybean, and wheat clones listed in Table 6 was determined. The BLASTX search using the EST sequences from clones listed in Table 7 revealed similarity of the polypeptides encoded by the cDNAs to RbohB and RbohD from Arabidopsis thaliana (NCBI General Identifier Nos. 3242783 and 3242789, respectively). Shown in Table 7 are the BLAST results for the sequences of the entire cDNA inserts comprising the indicated cDNA clones (“FIS”):

TABLE 7
BLAST Results for Sequences Encoding Polypeptides Homologous
to Arabidopsis thaliana RbohB and RbohD

| | | BLAST pLog Score | |
|---|---|---|---|
| Clone | Status | 3242783 (RbohB) | 3242789 (RbohD) |
| r1s2.pk0022.d7:fis | FIS | 123.00 | 127.00 |
| src2c.pk023.f15:fis | FIS | 60.15 | 62.40 |
| w11n.pk0054.d8:fis | FIS | 71.70 | 67.30 |

The data in Table 8 presents a calculation of the percent identity of the amino acid sequences set forth in SEQ ID NOs:14, 16, 18, 20, 22, 24, and 26 and the Arabidopsis thaliana RbohB and RbohD sequences (NCBI General Identifier Nos. 3242783 and 3242789, respectively).

TABLE 8
Percent Identity of Amino Acid Sequences Deduced From the Nucleotide
Sequences of cDNA Clones Encoding Polypeptides Homologous
to Arabidopsis thaliana RbohB and RbohD

Percent Identity to

| SEQ ID NO. | 3242783 (RbohB) | 3242789 (RbohD) |
|---|---|---|
| 14 | 60.5 | 58.7 |
| 16 | 73.7 | 69.7 |
| 18 | 70.1 | 57.6 |
| 20 | 52.2 | 47.8 |
| 22 | 63.9 | 63.3 |
| 24 | 42.3 | 42.3 |
| 26 | 65.8 | 58.4 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of a corn, a rice, a soybean, and a wheat RbohB.

### Example 5

### Characterization of cDNA Clones Encoding RbohC

The BLASTX search using the EST sequences from clones listed in Table 9 revealed similarity of the polypeptides encoded by the cDNAs to respiratory burst oxidase homolog C (RbohC) from Arabidopsis thaliana (NCBI General Identifier No.3242785). Shown in Table 9 are the BLAST results for individual ESTs (“EST”):

TABLE 9
BLAST Results for Sequences Encoding
Polypeptides Homologous to RbohC

| | | BLAST pLog Score |
|---|---|---|
| Clone | Status | 3242785 (Arabidopsis thaliana) |
| r1r6.pk0074.e9 | EST | 60.10 |

The sequence of the entire cDNA insert in the clone listed in Table 9 was determined. The BLASTX search using the EST sequences from clones listed in Table 10 revealed similarity of the polypeptides encoded by the cDNAs to RbohC from Arabidopsis thaliana (NCBI General Identifier No. 3242785). Shown in Table 10 are the BLAST results for the sequences of the entire cDNA insert comprising the indicated cDNA clone (“FIS”):

TABLE 10
BLAST Results for Sequences Encoding
Polypeptides Homologous RbohC

| | | BLAST pLog Score |
|---|---|---|
| Clone | Status | 3242785 (Arabidopsis thaliana) |
| r1r6.pk0074.e9:fis | FIS | 64.00 |

The data in Table 11 presents a calculation of the percent identity of the amino acid sequences set forth in SEQ ID NOs:28 and 30 and the Arabidopsis thaliana sequence (NCBI General Identifier No. 3242785).

TABLE 11
Percent Identity of Amino Acid Sequences Deduced From the
Nucleotide Sequences of cDNA Clones Encoding Polypeptides
Homologous to RbohC

| | Percent Identity to |
|---|---|
| SEQ ID NO. | 3242785 (Arabidopsis thaliana) |
| 28 | 59.8 |
| 30 | 60.9 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of a rice RbohC.

### Example 6

### Characterization of cDNA Clones Encoding RbohD

The BLASTX search using the EST sequences from clones listed in Table 12 revealed similarity of the polypeptides encoded by the cDNAs to respiratory burst oxidase homolog D (RbohD) from Arabidopsis thaliana (NCBI General Identifier No. 3242789). Shown in Table 12 are the BLAST results for individual ESTs (“EST”), or for the sequences of contigs assembled from two or more ESTs (“Contig”):

TABLE 12
BLAST Results for Sequences Encoding
Polypeptides Homologous to RbohD

| | | BLAST pLog Score |
|---|---|---|
| Clone | Status | 3242789 (Arabidopsis thaliana) |
| Contig of: | Contig | 106.00 |
| cco1n.pk055.115 | | |
| p0127.cntar92r | | |
| rr1.pk0004.a2 | EST | 56.05 |
| sr1.pk0073.f1 | EST | 61.40 |
| w1m96.pk044.g9 | EST | 41.00 |

The sequence of the entire cDNA insert in the rice, soybean, and wheat clones listed in Table 12 was determined. The BLASTX search using the EST sequences from clones listed in Table 13 revealed similarity of the polypeptides encoded by the cDNAs to RbohD from Arabidopsis thaliana (NCBI General Identifier No. 3242789). Shown in Table 13 are the BLAST results for the sequences of the entire cDNA inserts comprising the indicated cDNA clones (“FIS”):

TABLE 13
BLAST Results for Sequences Encoding Polypeptides
Homologous to RbohD

| | | BLAST pLog Score |
|---|---|---|
| Clone | Status | 3242789(Arabidopsis thaliana) |
| rr1.pk0004.a2:fis | FIS | >254.00 |
| sr1.pk0073.f1:fis | FIS | >254.00 |
| w1m96.pk044.g9:fis | FIS | >254.00 |

The data in Table 14 presents a calculation of the percent identity of the amino acid sequences set forth in SEQ ID NOs:32, 34, 36, 38, 40, 42, and 44 and the Arabidopsis thaliana sequence (NCBI General Identifier No. 3242789).

TABLE 14
Percent Identity of Amino Acid Sequences Deduced From the Nucleotide
Sequences of cDNA Clones Encoding Polypeptides Homologous to
RbohD

| | Percent Identity to |
|---|---|
| SEQ ID NO. | 3242789 (Arabidopsis/thaliana) |
| 32 | 64.5 |
| 34 | 75.8 |
| 36 | 63.5 |
| 38 | 51.0 |
| 40 | 73.7 |
| 42 | 66.1 |
| 44 | 71.1 |

Sequence alignments and percent identity calculations were performed using the Magalign program of the LASERGENE bioinformatics computing suite (DNASTAR Inc., Madison, Wis.) Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of a corn, a rice, a soybean, and a wheat RbohD.

### Example 7

### Characterization of cDNA Clones Encoding Respiratory Burst Oxidase Protein (Rboh)

The BLASTX search using the EST sequences from clones listed in Table 15 revealed similarity of the polypeptides encoded by the cDNAs to respiratory burst oxidase homolog (Rboh) from Arabidopsis thaliana and Oryza sativa (NCBI General Identifier Nos. 2654868 and 2654870 respectively). Shown in Table 15 are the BLAST results for individual ESTs (“EST”):

TABLE 15
BLAST Results for Sequences Encoding Polypeptides Homologous
to Respiratory Burst Oxidase Protein

| | | NCBI | BLAST |
|---|---|---|---|
| | | | pLog |
| Clone | Status | General Accession No. | Score |
| sdp2c.pk009.b13 | EST | 2654868 (Arabidopsis thaliana) | 50.70 |
| p0104.cabad88rb | EST | 2654870 (Oryza sativa) | 93.70 |
| rs11n.pk013.i4 | EST | 2654870 (Oryza sativa) | 60.22 |

The sequence of the entire cDNA insert in the clones listed in Table 15 was determined. The BLASTX search using the EST sequences from clones listed in Table 16 revealed similarity of the polypeptides encoded by the cDNAs to respiratory burst oxidase protein from Arabidopsis thaliana and Oryza sativa (NCBI General Identifier Nos. 7484893 and 7489460, respectively). The sequence having NCBI General Identifier No. 7484893 is 100% identical to the sequence having NCBI General Identifier No. 2654868, and the sequence having NCBI General Identifier No. 7489460 is 100% identical to the sequence having NCBI General Identifier No. 2654870. Shown in Table 16 are the BLAST results for the sequences of the entire cDNA inserts comprising the indicated cDNA clones (“FIS”):

TABLE 16
BLAST Results for Sequences Encoding Polypeptides Homologous
to Respiratory Burst Oxidase Protein

BLAST pLog Score

| | | 7484893 | 7489460 |
|---|---|---|---|
| Clone | Status | (A. thaliana) | (O. sativa) |
| p0104.cabad88rb:fis | FIS | >254.00 | >254.00 |
| rs11n.pk013.i4:fis | FIS | >254.00 | >254.00 |
| sdp2c.pk009.b13:fis | FIS | 72.52 | 68.00 |

The data in Table 17 presents a calculation of the percent identity of the amino acid sequences set forth in SEQ ID NOs:46, 48, 50, 52, 54, and 56 and the Arabidopsis thaliana and Oryza sativa sequences (NCBI General Identifier Nos. 7484893 and 7489460, respectively).

TABLE 17
Percent Identity of Amino Acid Sequences Deduced From the Nucleotide
Sequences of cDNA Clones Encoding Polypeptides Homologous
to Respiratory Burst Oxidase Protein

Percent Identity to

| SEQ ID NO. | 7484893 (A. thaliana) | 7489460 (O. sativa) |
|---|---|---|
| 46 | 62.3 | 81.9 |
| 48 | 65.5 | 91.8 |
| 50 | 100.0 | 92.3 |
| 52 | 75.5 | 93.7 |
| 54 | 73.7 | 91.7 |
| 56 | 88.8 | 83.9 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of a corn, a rice, and a soybean respiratory burst oxidase protein.

### Example 8

### Characterization of cDNA Clones Encoding Respiratory Burst Oxidase Homolog E (RbohE)

The BLASTX search using the EST sequences from clones listed in Table 18 revealed similarity of the polypeptides encoded by the cDNAs to RbohE from Arabidopsis thaliana (NCBI General Identifier No. 3242787). Shown in Table 18 are the BLAST results for individual ESTs (“EST”):

TABLE 18
BLAST Results for Sequences Encoding
Polypeptides Homologous to RbohE

| | | BLAST pLog Score |
|---|---|---|
| Clone | Status | 3242787 (Arabidopsis thaliana) |
| cen3n.pk0155.f12 | EST | 60.40 |
| se3.02c07 | EST | 18.70 |
| wr1.pk178.b5 | EST | 60.70 |

The sequence of the entire cDNA insert in the corn and wheat clones listed in Table 18 was determined. The BLASTX search using the EST sequences from clones listed in Table 19 revealed similarity of the polypeptides encoded by the cDNAs to RbohE from Arabidopsis thaliana (NCBI General Identifier No. 3242787). Shown in Table 19 are the BLAST results for the sequences of the entire cDNA inserts comprising the indicated cDNA clones (“FIS”):

TABLE 19
BLAST Results for Sequences Encoding Polypeptides
Homologous to RbohE

| | | BLAST pLog Score |
|---|---|---|
| Clone | Status | 3242787 (Arabidopsis thaliana) |
| cen3n.pk0155.f12:fis | FIS | 155.00 |
| wr1.pk178.b5:fis | FIS | 139.00 |

The data in Table 20 presents a calculation of the percent identity of the amino acid sequences set forth in SEQ ID NOs:58, 60, 62, 64, and 66 and the Arabidopsis thaliana sequence (NCBI General Identifier No. 3242787).

TABLE 20
Percent Identity of Amino Acid Sequences Deduced From the Nucleotide
Sequences of cDNA Clones Encoding Polypeptides Homologous to RbohE

| | Percent Identity to |
|---|---|
| SEQ ID NO. | 3242787 (Arabidopsis thaliana) |
| 58 | 74.4 |
| 60 | 33.6 |
| 62 | 72.1 |
| 64 | 62.2 |
| 66 | 61.8 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode subsitantial portions of a corn, a soybean, and a wheat RbohE.

### Example 9

### Characterization of cDNA Clones Encoding RbohF

The BLASTX search using the EST sequences from clones listed in Table 21 revealed similarity of the polypeptides encoded by the cDNAs to RbohF from Arabidopsis thaliana (NCBI General Identifier No. 3242456). Shown in Table 21 are the BLAST results for individual ESTs (“EST”):

TABLE 21
BLAST Results for Sequences Encoding
Polypeptides Homologous to RbohF

| | | BLAST pLog Score |
|---|---|---|
| Clone | Status | 3242456 (Arabidopsis thaliana) |
| p0010.cbpaa44rb | EST | 61.00 |
| sdp4c.pk014.k19 | EST | 22.10 |

The sequenced of the entire cDNA insert in the clones listed in Table 21 was determined. The BLASTX search using the EST sequences from clones listed in Table 22 revealed similarity of the polypeptides encoded by the cDNAs to phox homolog from Lycopersicon esculentum (NCBI General Identifier No. 4585142) and to RbohF from Arabidopsis thaliana (NCBI General Identifier No. 7484893). There is one amino acid difference (Thr to Ile at position 908) between the Arabidopsis thaliana sequences having NCBI General Identifier Nos. 3242456 and 7484893. Shown in Table 22 are the BLAST results for the sequences of the entire cDNA inserts comprising the indicated cDNA clones (“FIS”):

TABLE 22
BLAST Results for Sequences Encoding Polypeptides
Homologous to RbohF

BLAST pLog Score

| | | 4585142 | 7484893 |
|---|---|---|---|
| Clone | Status | (L. esculentum) | (A. thaliana) |
| p0010.cbpaa44rb:fis | FIS | >254.00 | >254.00 |
| sdp4c.pk014.k19:fis | FIS | 34.40 | 32.40 |

The data in Table 23 presents a calculation of the percent identity of the amino acid sequences set forth in SEQ ID NOs:68, 70, 72, and 74 and the Lycopersicon esculentum and Arabidopsis thaliana sequences (NCBI General Identifier Nos. 4585142 and 7484893, respectively).

TABLE 23
Percent Identity of Amino Acid Sequences Deduced From the Nucleotide
Sequences of cDNA Clones Encoding Polypeptides Homologous to RbohF

Percent Identity to

| SEQ ID NO. | 4585142 (L. esculentum) | 7484893 (A. thaliana) |
|---|---|---|
| 68 | 50.8 | 52.5 |
| 70 | 88.9 | 77.8 |
| 72 | 59.1 | 58.6 |
| 74 | 73.1 | 69.2 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of a corn and a soybean RbohF.

### Example 10

### Characterization of cDNA Clones Encoding tRNA-mnm⁵s²U-MT

The BLASTX search using the EST sequences from clones listed in Table 24 revealed similarity of the polypeptides encoded by the cDNAs to tRNA-mnm⁵s²U-MT from Borrelia burgdorferi (NCBI General Identifier No. 2688619). Shown in Table 24 are the BLAST results for individual ESTs (“EST”):

TABLE 24
BLAST Results for Sequences Encoding Polypeptides Homologous
to tRNA-mnm⁵s²U-MT

| | | BLAST pLog Score |
|---|---|---|
| Clone | Status | 2688619 (Borrelia burgdorferi) |
| cco1n.pk077.o18 | EST | 29.70 |
| se5.pk0029.d2 | EST | 11.10 |

The sequence of the entire cDNA insert in the clones listed in Table 24 was determined. The BLASTX search using the EST sequences from clones listed in Table 25 revealed similarity of the polypeptides encoded by the Contigs to a conserved hypothetical protein from Borrelia burgdorferi (NCBI General Identifier No. 2688619) and to a protein with similarities to tRNA-mnm⁵s²U-MT from Arabidopsis thaliana (NCBI General Identifier No. 4836940). Shown in Table 25 are the BLAST results for the sequences of the entire cDNA inserts comprising the indicated cDNA clones (“FIS”):

TABLE 25
BLAST Results for Sequences Encoding Polypeptides Homologous
to tRNA-mnm⁵s²U-MT

BLAST pLog Score

| Clone | Status | 2688619 | 4836940 |
|---|---|---|---|
| cco1n.pk077.o18:fis | FIS | 67.70 | 127.00 |
| se5.pk0029.d2:fis | FIS | 94.40 | >254.00 |

The data in Table 26 presents a calculation of the percent identity of the amino acid sequences set forth in SEQ ID NOs:76, 78, 80, and 82 and the Borrelia burgdorferi and Arabidopsis thaliana sequences (NCBI General Identifier Nos. 2688619 and 4836940. respectively).

TABLE 26
Percent Identity of Amino Acid Sequences Deduced From the Nucleotide
Sequences of cDNA Clones Encoding Polypeptides
Homologous to tRNA-mnm⁵s²U-MT

Percent Identity to

| SEQ ID NO. | 2688619 | 4836940 |
|---|---|---|
| 76 | 44.4 | 69.4 |
| 78 | 34.9 | 77.1 |
| 80 | 34.2 | 65.2 |
| 82 | 41.4 | 80.9 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of a corn and a soybean tRNA-mnm⁵s²U-MT.

### Example 11

### Characterization of cDNA Clones Encoding Chromomethylase

The BLASTX search using the EST sequences from clones listed in Table 27 revealed similarity of the polypeptides encoded by the contigs to chromomethylase from Arabidopsis thaliana (NCBI General Identifier Nos. 2865416 and 2865422) and from Arabidopsis arenosa (NCBI General Identifier No. 2766715). Shown in Table 27 are the BLAST results for individual ESTs (“EST”), or for the sequences of the entire cDNA inserts comprising the indicated cDNA clones (“FIS”):

TABLE 27
BLAST Results for Sequences Encoding Polypeptides Homologous
to Chromomethylase

BLAST pLog Score

| | | 2865416 | 2865422 | 2766715 |
|---|---|---|---|---|
| Clone | Status | (A. thaliana) | (A. thaliana) | (A. arenosa) |
| he11.pk0013.b1 | FIS | >254.00 | >254.00 | >254.00 |
| p0094.cssth92ra | EST | 32.15 | 31.22 | 32.40 |
| r10n.pk136.o14 | EST | 10.70 | 10.52 | 10.40 |
| w11n.pk0095.f3 | FIS | 73.70 | 72.70 | 71.70 |
| w1m0.pk0028.h3 | FIS | 9.40 | 9.40 | 3.30 |

The sequence of the entire CDNA insert in the clones listed in Table 27 was determined. The BLASTX search using the EST sequences from clones listed in Table 28 revealed similarity of the polypeptides encoded by the Contig to a putative chromomethylase from Arabidopsis thaliana (NCBI General Identifier No. 6665556) and by cDNAs to chromomethylases from Arabidopsis thaliana (NCBI General Identifier Nos. 2865422 and 2865416). Shown in Table 28 are the BLAST results for the sequences of the entire cDNA inserts comprising the indicated cDNA clones (“FIS”), or for the sequences of FISs encoding the entire protein (“CGS”):

TABLE 28
BLAST Results for Sequences Encoding Polypeptides
Homologous to Chromomethylase

| | | BLAST pLog Score | | |
|---|---|---|---|---|
| | | 6665556 | 2865422 | 2865416 |
| Clone | Status | (A. thaliana) | (A. thaliana) | (A. thaliana) |
| he11.pk0013.b1:fis | CGS | | >254.00 | >254.00 |
| p0094.cssth92ra:fis | FIS | 68.00 | 57.22 | 58.15 |
| r10n.pk136.o14:fis | FIS | 57.15 | 41.40 | 41.30 |
| srm.pk0035.c1:fis | FIS | 115.00 | 114.00 | 113.00 |

The data in Table 29 presents a calculation of the percent identity of the amino acid sequences set forth in SEQ ID NOs:84, 86, 88, 90, 92, 94, 96, 98, and 100 and the Arabidopsis thaliana sequences (NCBI General Identifier Nos. 6665556, 2865422, and 2865416).

TABLE 29
Percent Identity of Amino Acid Sequences Deduced From the Nucleotide
Sequences of cDNA Clones Encoding Polypeptides
Homologous to Chromomethylase

Percent Identity to

| | 6665556 | 2865422 | 2865416 |
|---|---|---|---|
| SEQ ID NO. | (A. thaliana) | (A. thaliana) | (A. thaliana) |
| 84 | 49.2 | 46.7 | 46.7 |
| 86 | 43.5 | 38.0 | 38.6 |
| 88 | 21.3 | 23.4 | 23.4 |
| 90 | 50.0 | 56.5 | 56.5 |
| 92 | 57.2 | 49.6 | 50.0 |
| 94 | 46.7 | 45.1 | 45.1 |
| 96 | 54.2 | 46.6 | 47.1 |
| 98 | 45.1 | 36.5 | 36.5 |
| 100 | 57.6 | 55.2 | 55.2 |

Sequence alignments and percent identity calculations were performed using the Megalign program of the LASERGENE bioinformatics computing suite (DNASTAR Inc., Madison, Wis.) Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of an artichoke, a corn, a rice, and two wheat chromomethylases and an entire artichoke chromomethylase.

### Example 12

### Characterization of cDNA Clones Encoding Cytosine 5-Methyltransferase

The BLASTX search using the EST sequences from clones listed in Table 30 revealed similarity of the polypeptides encoded by the cDNAs to cytosine 5-methyltransferase from Lycopersicon esculentum, Homo sapiens, Pisum sativum, or Schizosaccharomyces pombe (NCBI General Identifier Nos. 2887280, 4758184, 2654108, and 730347). Shown in Table 30 are the BLAST results for individual ESTs (“EST”), or for the sequences of the entire cDNA inserts comprising the indicated cDNA clones (“FIS”):

TABLE 30
BLAST Results for Sequences Encoding Polypeptides Homologous
to Cytosine 5-Methyltransferase

| | | | BLAST |
|---|---|---|---|
| Clone | Status | NCBI General Identifier No. | pLog Score |
| p0100.cbaaj24r | EST | 2887280 (L. esculentum) | 78.70 |
| rr1.pk0043.f8 | EST | 4758184 (Homo sapiens) | 12.70 |
| sgs2c.pk004.h13 | EST | 2654108 (Pisum sativum) | 105.00 |
| wr1.pk0076.a11 | EST | 2887280 (L. esculentum) | >254.00 |
| wre1n.pk0079.c6 | EST | 730347 (S. pombe) | 17.22 |

A corn sequence with similarities to cytosine 5-methyltransferases is found in the NCBI database having General Identifier No. 7489814. The sequence of the entire cDNA insert in the rice, soybean, and wheat clones listed in Table 30 was determined. The BLASTX search using the EST sequences from clones listed in Table 31 revealed similarity of the polypeptides encoded by the cDNAs to cytosine 5-methyltransferase from Homo sapiens, Pisum sativum, Zea mays, or Mus musculus (NCBI General Identifier Nos. 4758184, 7488824, 7489814, and 6753660, respectively). Shown in Table 31 are the BLAST results for the sequences of the entire cDNA inserts comprising the indicated cDNA clones (“FIS”):

TABLE 31
BLAST Results for Sequences Encoding Polypeptides Homologous
to Cytosine 5-Methyltransferase

| | | NCBI General | BLAST |
|---|---|---|---|
| Clone | Status | Identifier No. | pLog Score |
| rr1.pk0043.f8:fis | FIS | 4758184 (Homo sapiens) | 12.70 |
| sgs2c.pk004.h13:fis | FIS | 7488824 (Pisum sativum) | >254.00 |
| wr1.pk0076.a11:fis | FIS | 7489814 (Zea mays) | 180.00 |
| wre1n.pk0079.c6:fis | FIS | 6753660 (Mus musculus) | 63.52 |

The data in Table 32 presents a calculation of the percent identity of the amino acid sequences set forth in SEQ ID NOs:102, 104, 106, 108, 110, 112, 114, 116, and 118 and the Homo sapiens, Pisum sativum, Zea mays, or Mus musculus sequences (NCBI General Identifier Nos. 4758184, 7488824, 7489814, and 6753660).

TABLE 32
Percent Identity of Amino Acid Sequences Deduced From the
Nucleotide Sequences
Sequences of cDNA Clones Encoding Polypeptides
Homologous to Cytosine 5-Methyltransferase

| | Percent Identity to | | | |
|---|---|---|---|---|
| | 4758184 | 7488824 | 7489814 | 6753660 |
| SEQ ID NO. | (H. sapiens) | (P. sativum) | (Z. mays) | (M. musculus) |
| 102 | 14.3 | 77.1 | 97.1 | 14.9 |
| 104 | 39.8 | 21.7 | 20.5 | 39.8 |
| 106 | 19.9 | 88.1 | 77.8 | 16.5 |
| 108 | 13.8 | 81.5 | 92.2 | 12.5 |
| 110 | 13.8 | 81.5 | 92.2 | 12.5 |
| 112 | 37.1 | 22.5 | 19.1 | 37.1 |
| 114 | 13.8 | 91.2 | 82.8 | 13.2 |
| 116 | 13.6 | 80.5 | 91.3 | 12.4 |
| 118 | 33.7 | 12.1 | 12.1 | 35.3 |

Sequence alignments and percent identity calculations were performed using the Megalign program of the LASERGENE bioinformatics computing suite (DNASTAR Inc., Madison, Wis.) Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of corn, rice, soybean, and wheat cytosine 5-methyltransferases.

### Example 13

### Characterization of cDNA Clones Encoding Phospholipase Dα

The BLASTX search using the EST sequences from clones listed in Table 33 revealed similarity of the polypeptides encoded by the cDNAs to Phospholipase Dα (PLDα) from Vigna unguiculata and Zea mays (NCBI General Identifier Nos. 3914359 and 2499708, respectively). Shown in Table 33 are the BLAST results for individual ESTs (“EST”):

TABLE 33
BLAST Results for Sequences Encoding Polypeptides Homologous
to Phospholipase Dα

| | | | BLAST |
|---|---|---|---|
| Clone | Status | NCBI General Identifier No. | pLog Score |
| sgs4c.pk004.c18 | EST | 3914359 (Vigna unguiculata) | 76.00 |
| wlk4.pk0022.b7 | EST | 2499708 (Zea mays) | 15.52 |

The sequence of the entire cDNA insert in the clones listed in Table 33 was determined. The BLASTP search using the amino acid sequences derived from clones listed in Table 34 revealed similarity of the polypeptides encoded by the cDNAs to PLD α from Vigna unguiculata and Oryza sativa (NCBI General Identifier Nos. 3914359 and 2499709, respectively). Shown in Table 34 are the BLAST results for the amino acid sequence of the entire protein derived from the sequences of the entire CDNA insert comprising the indicated CDNA clones(“CGS”):

TABLE 34
BLAST Results for Sequences Encoding Polypeptides
Homologous to Phospholipase Dα

| | | NCBI | BLAST |
|---|---|---|---|
| Clone | Status | General Identifier No. | pLog Score |
| sfl1.pk128.a18:fis | CGS | 3914359 (Vigna | >254.00 |
| | | unguiculata) | |
| wlk4.pk0022.b7:fis | CGS | 2499709 (Oryza sativa) | >254.00 |

The data in Table 35 presents a calculation of the percent identity of the amino acid sequences set forth in SEQ ID NOs:120, 122, 124, and 126 and the Vigna unguiculata and Oryza sativa sequences (NCBI General Identifier Nos. 3914359 and 2499709, respectively).

TABLE 35
Percent Identity of Amino Acid Sequences Deduced From the Nucleotide
Sequences of cDNA Clones Encoding Polypeptides
Homologous to Phospholipase Dα

Percent Identity to

| SEQ ID NO. | 3914359 (V. unguiculata) | 2499709 (Oryza sativa) |
|---|---|---|
| 120 | 87.2 | 67.7 |
| 121 | 36.2 | 43.6 |
| 122 | 90.1 | 79.5 |
| 124 | 79.0 | 89.7 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant CDNA clones encode a substantial portion and an entire soybean and wheat phospholipase D(Xs.

### Example 14

### Characterization of cDNA Clones Encoding Phospholipase Dγ

The BLASTX search using the EST sequences from clones listed in Table 36 revealed similarity of the polypeptides encoded by the cDNAs to Phospholipase Dγ from Arabidopsis thaliana (NCBI General Identifier No. 2653885). Shown in Table 36 are the BLAST results for individual ESTs (“EST”):

TABLE 36
BLAST Results for Sequences Encoding Polypeptides
Polypeptides to Phospholipase Dγ

| | | BLAST pLog Score |
|---|---|---|
| Clone | Status | 2653885 (Arabidopsis thaliana) |
| p0083.cldaz07r | EST | 48.52 |
| src3c.pk012.d7 | EST | 41.00 |

The sequence of the entire cDNA insert in the clones listed in Table 36 was determined. The BLASTP search using the amino acid sequences derived from clones listed in Table 37 revealed similarity of the polypeptides encoded by the Contig to phospholipase D from Arabidopsis thaliana (NCBI General Identifier No. 1871182) and by cDNAs to Phospholipase Dγ from Nicotiana tabacum or Gossypium hirsutum (NCBI General Identifier Nos. 6180159 and 5442428, respectively). Shown in Table 37 are the BLAST results for the sequences encoded by the entire cDNA inserts comprising the indicated cDNA clones (“FIS”), or by the sequences of the entire protein encoded by the indicated FIS(“CGS”):

TABLE 37
BLAST Results for Sequences Encoding Polypeptides Homologous
Polypeptides to Phospholipase Dγ

| | | | BLAST | |
|---|---|---|---|---|
| | | | pLog Score | |
| | | 6180159 | 1871182 | 5442428 |
| | | (N. | (A. | (G. |
| Clone | Status | tabacum) | thaliana) | hirsutum) |
| p0083.cldaz07r:fis | FIS | 54.05 | | 52.22 |
| src3c.pk012.d7:fis | CGS | | >254.00 | >254.00 |

The data in Table 38 presents a calculation of the percent identity of the amino acid sequences set forth in SEQ ID NOs:128, 130, 132, and 134 and the Nicotiana tabacum and Gossypium hirsutum sequences (NCBI General Identifier Nos. 6180159 and 5442428, respectively).

TABLE 38
Percent Identity of Amino Acid Sequences Deduced From the
Nucleotide Sequences
Sequences of cDNA Clones Encoding Polypeptides
Homologous to Phospholipase Dγ

Percent Identity to

| SEQ ID NO. | 6180159 (N. tabacum) | 5442428 (G. hirsutum) |
|---|---|---|
| 128 | 78.4 | 77.6 |
| 130 | 11.3 | 54.0 |
| 132 | 79.2 | 76.0 |
| 134 | 72.6 | 69.1 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portion of a corn Phospholipase Dγ and a substantial portion and an entire soybean Phospholipase Dγ.

### Example 15

### Characterization of cDNA Clones Encoding TF IIF α Subunit

The BLASTX search using the EST sequences from clone listed in Table 39 revealed similarity of the polypeptides encoded by the cDNAs to transcription factor IIF α subunit (TF IIF α subunit) from Xenopus laevis (NCBI General Identifier No. 464522). Shown in Table 39 are the BLAST results for individual ESTs (“EST”):

TABLE 39
BLAST Results for Sequences Encoding Polypeptides
Homologous to TF IIF α Subunit

| | | BLAST pLog Score |
|---|---|---|
| Clone | Status | 464522 (Xenopus laevis) |
| p0026.ccrbd22r | EST | 5.00 |

The sequence of the entire cDNA insert in the clone listed in Table 39 was determined. The BLASTP search using the amino acid sequences derived from clone listed in Table 40 revealed similarity of the polypeptides encoded by the Contig to a putative protein with similarities to TF IIF α subunit from Arabidopsis thaliana (NCBI General Identifier No. 5823572) and by the cDNAs to TF IIF ax subunit from Xenopus laevis (NCBI General Identifier No. 464522). Shown in Table 40 are the BLAST results for the amino acid sequences derived from the entire cDNA inserts comprising the indicated cDNA clone (“FIS”):

TABLE 40
BLAST Results for Sequences Encoding Polypeptides
Homologous to TF IIF α Subunit

| | | BLAST pLog Score |
|---|---|---|
| Clone | Status | 464522 (Xenopus laevis) |
| p0026.ccrbd22r:fis | FIS | 22.00 |

The data in Table 41 presents a calculation of the percent identity of the amino acid sequences set forth in SEQ ID NOs: 136 and 138 and the Xenopus laevis and Arabidopsis thaliana sequences (NCBI General Identifier Nos. 464522 and 5823572, respectively).

TABLE 41
Percent Identity of Amino Acid Sequences Deduced From the Nucleotide
Sequences of cDNA Clones Encoding Polypeptides
Homologous to TF IIF α Subunit

| | Percent Identity to | |
|---|---|---|
| SEQ ID NO. | 464522 (Xenopus laevis) | 5823572 (A. thaliana) |
| 136 | 22.9 | 65.1 |
| 138 | 17.2 | 55.8 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of a corn TF IIF α subunit.

### Example 16

### Characterization of cDNA Clones Encoding TF IIF β Subunits

The BLASTX search using the EST sequences from clones listed in Table 42 revealed similarity of the polypeptides encoded by the cDNAs to TF IIF β subunit from Schizosaccharomyces pombe (NCBI General Identifier No. 4049502). Table 42 are the BLAST results for individual ESTs (“EST”):

TABLE 42
BLAST Results for Sequences Encoding Polypeptides
Homologous to TF IIF β Subunit

| | | BLAST pLog Score |
|---|---|---|
| Clone | Status | 4049502 (Schizosaccharomyces pombe) |
| p0014.ctusq39r | EST | 11.70 |
| w1m24.pk0018.g9 | EST | 9.30 |

The sequence of the entire cDNA insert in the clones listed in Table 42 was determined. Further sequencing and searching of the DuPont proprietary database allowed the identification of other corn and rice clones encoding TF IIF β subunit. The BLASTX search using the EST sequences from clones listed in Table 43 revealed similarity of the polypeptides encoded by the cDNAs to TF IIF β subunit from Schizosaccharomyces pombe (NCBI General Identifier No. 7493495). The amino acid sequences having NCBI General Identifier No. 4049502 and NCBI General Identifier No. 7493495 are 100% identical. Shown in Table 43 are the BLAST results for the sequences of the entire cDNA inserts comprising the indicated cDNA clones (“FIS”), or for the sequences of contigs assembled from an FIS and one or more ESTs (“Contig”):

TABLE 43
BLAST Results for Sequences Encoding Polypeptides
Homologous to TF IIF β Subunit

| | | BLAST pLog Score |
|---|---|---|
| Clone | Status | 7493495 (Schizosaccharomyces pombe) |
| Contig of: | Contig | 15.30 |
| p0014.ctusq39r:fis | | |
| p0107.cbcap19r | | |
| rca1n.pk007.p13:fis | FIS | 12.15 |
| r10n.pk0063.e10:fis | FIS | 18.70 |
| r1s6.pk0059.b8:fis | FIS | 18.22 |
| w1m24.pk0018.g9:fis | FIS | 10.70 |

The data in Table 44 presents a calculation of the percent identity of the amino acid sequences set forth in SEQ ID NOs:140, 142, 144, 146, 148, 150, and 152 and the Schizosaccharomyces pombe sequence (NCBI General Identifier No. 7493495).

TABLE 44
Percent Identity of Amino Acid Sequences Deduced From the Nucleotide
Sequences of cDNA Clones Encoding Polypeptides
Homologous to TF IIF β Subunit

| | Percent Identity to |
|---|---|
| SEQ ID NO. | 7493495 (Schizosaccharomyces pombe) |
| 140 | 38.4 |
| 142 | 45.6 |
| 144 | 24.9 |
| 146 | 34.5 |
| 148 | 23.2 |
| 150 | 21.7 |
| 152 | 42.9 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode substantial portions of a corn TF IIF β subunit.

### Example 17

### Characterization of cDNA Clones Encoding Asparaginyl-tRNA Synthetase

The BLASTX search using the EST sequences from clones listed in Table 45 revealed similarity of the polypeptides encoded by the cDNAs to asparaginyl-tRNA synthetases from Arabidopsis thaliana (NCBI General Identifier No. 2664210). Shown in Table 45 are the BLAST results for individual ESTs (“EST”), for the sequences of the entire cDNA inserts comprising the indicated cDNA clones (“FIS”), or for FISs encoding the entire protein (“CGS”):

TABLE 45
BLAST Results for Sequences Encoding Polypeptides Homologous
to Asparaginyl-tRNA Synthetase

| | | BLAST pLog Score |
|---|---|---|
| Clone | Status | 2664210 (Arabidopsis thaliana) |
| p0119.cmtne90r:fis | CGS | 130.00 |
| r10n.pk0039.b7:fis | FIS | 141.00 |
| src1c.pk001.a5:fis | CGS | >254.00 |
| wdr1.pk0005.f7:fis | FIS | 24.70 |
| wr1.pk0067.h2 | EST | 20.30 |

The data in Table 46 presents a calculation of the percent identity of the amino acid sequences set forth in SEQ ID NOs:154, 156, 158, 160, and 162 and the Arabidopsis thaliana sequence (NCBI General Identifier No. 2664210).

TABLE 46
Percent Identity of Amino Acid Sequences Deduced From the Nucleotide
Sequences of cDNA Clones Encoding Polypeptides Homologous
to Asparaginyl-tRNA Synthetase

| | Percent Identity to |
|---|---|
| SEQ ID NO. | 2664210 (Arabidopsis thaliana) |
| 154 | 44.0 |
| 156 | 86.4 |
| 158 | 72.4 |
| 160 | 87.7 |
| 162 | 36.7 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode a substantial portion of one rice and two wheat asparaginyl-tRNA synthetase, one entire corn, and one entire soybean asparaginyl-tRNA synthetase.

### Example 18

### Characterization of cDNA Clones Encoding Glutaminyl-tRNA Synthetase

The BLASTX search using the EST sequences from clones listed in Table 47 revealed similarity of the polypeptides encoded by the cDNAs to glutaminyl-tRNA synthetase from Lupinus luteus (NCBI General Identifier No. 3915866). Shown in Table 47 are the BLAST results for the sequences of the entire cDNA inserts comprising the indicated cDNA clones (“FIS”):

TABLE 47
BLAST Results for Sequences Encoding Polypeptides Homologous
to Glutaminyl-tRNA Synthetase

| | | BLAST pLog Score |
|---|---|---|
| Clone | Status | 3915866 (Lupinus luteus) |
| p0129.c1mad36r:fis | FIS | >254.00 |
| rds1c.pk007.e9:fis | FIS | >254.00 |
| sic1c.pk001.e18:fis | FIS | 61.15 |
| w1mk1.pk0001.g6:fis | FIS | >254.00 |

The data in Table 48 presents a calculation of the percent identity of the amino acid sequences set forth in SEQ ID NOs:164, 166, 168, and 170 and the Lupinus luteus sequence (NCBI General Identifier No. 3915866).

TABLE 48
Percent Identity of Amino Acid Sequences Deduced From the Nucleotide
Sequences of cDNA Clones Encoding Polypeptides Homologous
to Glutaminyl-tRNA Synthetase

| | Percent Identity to |
|---|---|
| SEQ ID NO. | 3915866 (Lupinus luteus) |
| 164 | 76.9 |
| 166 | 80.0 |
| 168 | 92.0 |
| 170 | 77.0 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode a substantial portion of a corn, a rice, a soybean, and a wheat glutaminyl-tRNA synthetase.

### Example 19

### Characterization of cDNA Clones Encoding EDS1

The BLASTX search using the EST sequences from clones listed in Table 49 revealed similarity of the polypeptides encoded by the cDNAs to EDS1 from Arabidopsis thaliana (NCBI General Identifier No. 4454567). Shown in Table 49 are the BLAST results for the sequences of the entire CDNA inserts comprising the indicated CDNA clones (“FIS”), or the sequences of FISs encoding the entire protein (“CGS”):

TABLE 49
BLAST Results for Sequences Encoding Polypeptides
Homologous to EDS1

| | | BLAST pLog Score |
|---|---|---|
| Clone | Status | 4454567 (Arabidopsis thaliana) |
| r10n.pk127.m10:fis | FIS | 63.30 |
| s1s2c.pk037.c11:fis | CGS | 126.00 |
| wre1n.pk160.d1:fis | FIS | 87.52 |

The data in Table 50 presents a calculation of the percent identity of the amino acid sequences set forth in SEQ ID NOs:172, 174, and 176 and the Arabidopsis thaliana sequence (NCBI General Identifier No. 4454567).

TABLE 50
Percent Identity of Amino Acid Sequences Deduced From the Nucleotide
Sequences of cDNA Clones Encoding Polypeptides
Homologous to EDS1

| | Percent Identity to |
|---|---|
| SEQ ID NO. | 4454567 (Arabidopsis thaliana) |
| 172 | 34.6 |
| 174 | 37.4 |
| 176 | 37.4 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant CDNA clones encode a substantial portion of a rice and a wheat EDS1 and an entire soybean EDS1.

### Example 20

### Characterization of cDNA Clones Encoding AP50

The BLASTX search using the EST sequences from clones listed in Table 51 revealed similarity of the polypeptides encoded by the cDNAs to AP50 from Arabidopsis thaliana (NCBI General Identifier No. 2271477). Shown in Table 51 are the BLAST results for individual ESTs (“EST”), for the sequences of the entire cDNA inserts comprising the indicated cDNA clones (“FIS”), or for the sequences of FISs encoding an entire protein (“CGS”):

TABLE 51
BLAST Results for Sequences Encoding Polypeptides
Homologous to AP50

| | | BLAST pLog Score |
|---|---|---|
| Clone | Status | 2271477 (Arabidopsis thaliana) |
| p0127.cntam18r | EST | 79.15 |
| r1r6.pk0083.e10:fis | FIS | 81.40 |
| sdp3c.pk006.d23:fis | CGS | >254.00 |
| wdk1c.pk012.n13:fis | FIS | 35.15 |

The data in Table 52 presents a calculation of the percent identity of the amino acid sequences set forth in SEQ ID NOs:178, 180, 182, and 184 and the Arabidopsis thaliana sequence (NCBI General Identifier No. 2271477).

TABLE 52
Percent Identity of Amino Acid Sequences Deduced From the Nucleotide
Sequences of cDNA Clones Encoding Polypeptides Homologous to AP50

| | Percent Identity to |
|---|---|
| SEQ ID NO. | 2271477 (Arabidopsis thaliana) |
| 178 | 80.0 |
| 180 | 88.9 |
| 182 | 94.3 |
| 184 | 88.5 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode a substantial portion of a corn, a rice, and a wheat AP50 and an entire soybean AP50.

### Example 21

### Characterization of cDNA Clones Encoding Alpha Adaptin

The BLASTX search using the EST sequences from clones listed in Table 53 revealed similarity of the polypeptides encoded by the cDNAs to alpha adaptin from Mus musculus or Drosophila melanogaster (NCBI General Identifier No. 6671561 and 7296210, respectively). Shown in Table 53 are the BLAST results for the sequences of the entire cDNA inserts comprising the indicated cDNA clones (“FIS”), or for the sequences of FISs encoding an entire protein (“CGS”):

TABLE 53
BLAST Results for Sequences Encoding Polypeptides
Homologous to Alpha Adaptin

| | | NCBI | BLAST |
|---|---|---|---|
| Clone | Status | General Identifier No. | pLog Score |
| p0119.cmtoj48r:fis | CGS | 6671561 (Mus musculus) | >254.00 |
| s12.pk121.m20:fis | FIS | 7296210 (D. melanogaster) | 29.00 |

The data in Table 54 presents a calculation of the percent identity of the amino acid sequences set forth in SEQ ID NOs:186 and 188 and the Mus musculus and Drosophila melanogaster sequences (NCBI General Identifier No. 6671561 and 7296210, respectively).

TABLE 54
Percent Identity of Amino Acid Sequences
Deduced From the Nucleotide Sequences of cDNA Clones
Encoding Polypeptides Homologous to Alpha Adaptin

| | Percent Identity to | |
|---|---|---|
| SEQ ID NO. | 6671561 (Mus musculus) | 7296210 (D. melanogaster) |
| 186 | 31.5 | 35.1 |
| 188 | 18.2 | 19.6 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments, BLAST scores and probabilities indicate that the nucleic acid fragments comprising the instant cDNA clones encode a substantial portion of a soybean and an entire corn alpha adaptin.

### Example 22

### Characterization of cDNA Clones Encoding Beta' Adaptin

The BLASTX search using the EST sequences from clones listed in Table 55 revealed similarity of the polypeptides encoded by the cDNAs to beta' adaptin from Arabidopsis thaliana, Drosophila melanogaster, and/or Homo sapiens (NCBI General Identifier Nos. 7441349, 481762, and 1532118, respectively). Shown in Table 55 are the BLAST results for individual ESTs (“EST”), for the sequences of the entire cDNA inserts comprising the indicated cDNA clones (“FIS”), or for the sequences of FISs encoding an entire protein (“CGS”):

TABLE 55
BLAST Results for Sequences Encoding Polypeptides
Homologous to Beta′ Adaptin

| | | BLAST pLog Score | | |
|---|---|---|---|---|
| | | | 481762 | 1532118 |
| | | 7441349 | (D. melano- | (Homo |
| Clone | Status | (A. thaliana) | gaster) | sapiens) |
| p0119.cmtnr87r:fis | CGS | >254.00 | >254.00 | >254.00 |
| rds1c.pk005.c17:fis | FIS | >254.00 | 176.00 | 174.00 |
| sls2c.pk005.m4:fis | FIS | | 113.00 | 111.00 |
| wkm2c.pk0002.a3 | EST | | 11.40 | 15.15 |

The data in Table 56 presents a calculation of the percent identity of the amino acid sequences set forth in SEQ ID NOs:190, 192, 194, and 196 and the Arabidopsis thaliana, Drosophila melanogaster, and Homo sapiens sequence (NCBI General Identifier Nos. 7441349, 481762, and 1532118, respectively).

TABLE 56
Percent Identity of Amino Acid Sequences Deduced From
the Nucleotide Sequences of cDNA Clones Encoding Polypeptides
Homologous to Beta′ Adaptin

| | Percent Identity to | | |
|---|---|---|---|
| | 7441349 | 481762 | 1532118 |
| SEQ ID NO. | (A. thaliana) | (D. melanogaster) | (Homo sapiens) |
| 190 | 79.2 | 47.4 | 47.6 |
| 192 | 79.5 | 49.0 | 49.8 |
| 194 | 43.1 | 46.0 | 45.3 |
| 196 | 69.0 | 31.9 | 37.9 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=-3, WINDOW=5 and DIAGONALS SAVED=5. Sequence alignments, BLAST scores and probabilities indicate that the nucleic acid fragments comprising the instant cDNA clones encode a substantial portion of a rice, a soybean, and a wheat beta' adaptin and an entire corn beta' adaptin.

### Example 23

### Expression of Chimeric Genes in Monocot Cells

A chimeric gene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML 103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML 103. Plasmid pML 103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega; Madison, Wis.). Vector and insert DNA can be ligated at 15° C. overnight, essentially as described (Maniatis). The ligated DNA may then be used to transform E. coli XL1-Blue (Epicurian Coli XL-l Blue™; Stratagene, La Jolla, Calif.). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric gene encoding, in the 5′ to 3′ direction, the maize 27 kD zein promoter, a cDNA fragment encoding the instant polypeptides, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten pg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of mercury (Hg). The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### Example 24

### Expression of Chimeric Genes in Dicot Cells

A seed-specific construct composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant polypeptides in transformed soybean. The phaseolin construct includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire construct is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC 18 vector carrying the seed construct.

Soybean embryos may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL of liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic™ PDS 1000/HE instrument (helium retrofit) can be used for these transformations.

A selectable marker gene which can be used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al.(1983) Gene 25:179-188) and the 3′ region of the nopaline synthase gene from the T-DNA of the Ti plasmid of Agrobacterium tumefaciens. The seed construct comprising the phaseolin 5′ region, the fragment encoding the instant polypeptides and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches of mercury (Hg). The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 25

### Expression of Chimeric Genes in Microbial Cells

The cDNAs encoding the instant polypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% low melting agarose gel. Buffer and agarose contain 10 μg/mL ethidium bromide for visualization of the DNA fragment. The fragment can then be purified from the agarose gel by digestion with GELase™ (Epicentre Technologies, Madison, Wis.) according to the manufacturer's instructions, ethanol precipitated, dried and resuspended in 20 μL of water. Appropriate oligonucleotide adapters may be ligated to the fragment using T4 DNA ligase (New England Biolabs (NEB), Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptides are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25° C. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

### Example 26

### Evaluating Compounds for Their Ability to Inhibit the Activity of tRNA Methyltransferases or Aminoacyl-tRNA Synthetases

The polypeptides described herein may be produced using any number of methods known to those skilled in the art. Such methods include, but are not limited to, expression in bacteria as described in Example 25, or expression in eukaryotic cell culture, in planta, and using viral expression systems in suitably infected organisms or cell lines. The instant polypeptides may be expressed either as mature forms of the proteins as observed in vivo or as fusion proteins by covalent attachment to a variety of enzymes, proteins or affinity tags. Common fusion protein partners include glutathione S-transferase (“GST”), thioredoxin (“Trx”), maltose binding protein, and C- and/or N-terminal hexahistidine polypeptide (“(His)₆”). The fusion proteins may be engineered with a protease recognition site at the fusion point so that fusion partners can be separated by protease digestion to yield intact mature enzyme. Examples of such proteases include thrombin, enterokinase and factor Xa. However, any protease can be used which specifically cleaves the peptide connecting the fusion protein and the enzyme.

Purification of the instant polypeptides, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. When the instant polypeptides are expressed as fusion proteins, the purification protocol may include the use of an affinity resin which is specific for the fusion protein tag attached to the expressed enzyme or an affinity resin containing ligands which are specific for the enzyme.

For example, the instant polypeptides may be expressed as a fusion protein coupled to the C-terminus of thioredoxin. In addition, a (His)₆ peptide may be engineered into the N-terminus of the fused thioredoxin moiety to afford additional opportunities for affinity purification. Other suitable affinity resins could be synthesized by linking the appropriate ligands to any suitable resin such as Sepharose-4B. In an alternate embodiment, a thioredoxin fusion protein may be eluted using dithiothreitol; however, elution may be accomplished using other reagents which interact to displace the thioredoxin from the resin. These reagents include β-mercaptoethanol or other reduced thiol. The eluted fusion protein may be subjected to further purification by traditional means as stated above, if desired. Proteolytic cleavage of the thioredoxin fusion protein and the enzyme may be accomplished after the fusion protein is purified or while the protein is still bound to the ThioBond™ affinity resin or other resin.

Crude, partially purified or purified enzyme, either alone or as a fusion protein, may be utilized in assays for the evaluation of compounds for their ability to inhibit enzymatic activation of the instant polypeptides disclosed herein. Assays may be conducted under well-known experimental conditions which permit optimal enzymatic activity. For example, detection of altered activities of the introduced tRNA-mnm⁵s²U-MT would be performed in bacterial deletion backgrounds. The methods could be similar to, but not limited to, those presented in Elseviers et al. (1984) Nucleic Acids Res. 12:3521-3534 or Hagervall and Bjork (1984) Mol. Gen. Genet. 196:194-200. Assays for aminoacyl t-RNA synthetases are presented by Lloyd et al. (1995) Nucleic Acids Res. 23:2886-2892.

What is claimed is:

1. An isolated nucleic acid comprising a nucleotide sequence selected from the group consisting of:
(a) an isolated nucleic acid encoding a polypeptide selected from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196; and
(b) an isolated nucleic acid sequence comprising a complement of (a).

2. An isolated polynucleotide comprising a nucleotide sequence selected from the group consisting of:
(a) a first nucleotide sequence encoding a polypeptide of at least 80 amino acids that has at least 92% identity based on the Clustal method of alignment when compared to a polypeptide selected from the group consisting of SEQ ID NOs:120, 122, 124, 126, 128, 130, 132, and 134; and
(b) a second nucleotide sequence comprising a complement of the first nucleotide sequence.

3. The isolated polynucleotide of claim 2, wherein the first nucleotide sequence comprises of a nucleic acid sequence selected from the group consisting of SEQ ID NOs:119, 121, 123, 125, 127, 129, 131, and 133.

4. The isolated polynucleotide of claim 2 wherein the nucleotide sequences are DNA.

5. The isolated polynucleotide of claim 2 wherein the nucleotide sequences are RNA.

6. A chimeric gene comprising the isolated polynucleotide of claim 2 operably linked to at least one suitable regulatory sequence.

7. A host cell comprising the chimeric gene of claim 6.

8. A host cell comprising the isolated polynucleotide of claim 2.

9. The host cell of claim 8 wherein the host cell is selected from the group consisting of yeast, bacteria, and plant.

10. A virus comprising the isolated polynucleotide of claim 2.

11. A polypeptide of at least 80 amino acids that has at least 92% identity based on the Clustal method of alignment when compared to a polypeptide selected from the group consisting of SEQ ID NOs:120, 122, 124, 126, 128, 130, 132, and 134.

12. A method of selecting an isolated polynucleotide that affects the level of expression of a phospholipase D polypeptide in a plant cell, the method comprising the steps of:
(a) constructing an isolated polynucleotide comprising a nucleotide sequence of at least one of 30 contiguous nucleotides derived from an isolated polynucleotide of claim 2;
(b) introducing the isolated polynucleotide into the plant cell;
(c) measuring the level of the polypeptide in the plant cell containing the polynucleotide; and
(d) comparing the level of the polypeptide in the plant cell containing the isolated polynucleotide with the level of the polypeptide in a plant cell that does not contain the isolated polynucleotide.

13. The method of claim 12 wherein the isolated polynucleotide consists of a nucleotide sequence selected from the group consisting of SEQ ID NOs:119, 121, 123, 125, 127, 129, 131, and 133.

14. A method of selecting an isolated polynucleotide that affects the level of expression of a phospholipase D polypeptide in a plant cell, the method comprising the steps of:
(a) constructing the isolated polynucleotide of claim 2;
(b) introducing the isolated polynucleotide into the plant cell;
(c) measuring the level of the polypeptide in the plant cell containing the polynucleotide; and
(d) comparing the level of the polypeptide in the plant cell containing the isolated polynucleotide with the level of the polypeptide in a plant cell that does not contain the polynucleotide.

15. A method of obtaining a nucleic acid fragment encoding a phospholipase D polypeptide comprising the steps of:
(a) synthesizing an oligonucleotide primer comprising a nucleotide sequence of at least one of 30 contiguous nucleotides derived from a nucleotide sequence selected from the group consisting of SEQ ID NOs:119, 121, 123, 125, 127, 129, 131, and 133 and a complement of such nucleotide sequences; and
(b) amplifying a nucleic acid sequence using the oligonucleotide primer.

16. A method of obtaining a nucleic acid fragment encoding a phospholipase D polypeptide comprising the steps of:
(a) probing a cDNA or genomic library with an isolated polynucleotide comprising at least one of 30 contiguous nucleotides derived from a nucleotide sequence selected from the group consisting of SEQ IDNOs:119, 121, 123, 125, 127, 129, 131, and 133 and a complement of such nucleotide sequences;
(b) identifying a DNA clone that hybridizes with the isolated polynucleotide;
(c) isolating the identified DNA clone; and
(d) sequencing a cDNA or genomic fragment that comprises the isolated DNA clone.

17. A composition comprising the isolated polynucleotide of claim 2.

18. A composition comprising the polypeptide of claim 11.

19. An isolated polynucleotide of claim 2 comprising a nucleotide sequence having at least one of 30 contiguous nucleotides.

20. A method for positive selection of a transformed cell comprising the steps of:
(a) transforming a host cell with the chimeric gene of claim 6; and
(b) growing the transformed host cell under conditions which allow expression of a polynucleotide in an amount sufficient to complement a null mutant to provide a positive selection means.

21. The method of claim 20 wherein the host cell is a plant.

22. The method of claim 21 wherein the plant cell is a monocot.

23. The method of claim 21 wherein the plant cell is a dicot.

24. A method of altering the level of expression of a phospholipase D in a host cell comprising the steps of:
(a) transforming a host cell with the chimeric gene of claim 6; and
(b) growing the transformed host cell produced in step (a) under conditions that are suitable for expression of the chimeric gene
wherein expression of the chimeric gene results in production of altered levels of a phospholipase D in the transformed host cell.


SEQUENCE LISTING (196 sequences)

SEQ ID NO: 1 - DNA, 688 bp, Zea mays
  unsure (49)
  unsure (78)
  unsure (125)
  unsure (137)
  unsure (163)
  unsure (191)
  unsure (194)
  unsure (226)
  unsure (296)
  unsure (379)
  unsure (384)..(385)
  unsure (389)
  unsure (395)
  unsure (416)
  unsure (424)
  unsure (428)
  unsure (446)
  unsure (461)
  unsure (473)
  unsure (500)..(501)
  unsure (522)
  unsure (527)
  unsure (533)..(534)
  unsure (540)
  unsure (549)
  unsure (554)
  unsure (563)
  unsure (565)
  unsure (572)
  unsure (580)
  unsure (586)
  unsure (589)
  unsure (594)..(595)
  unsure (606)
  unsure (611)
  unsure (614)
  unsure (623)..(624)
  unsure (627)..(628)
  unsure (630)..(631)
  unsure (641)
  unsure (667)
  unsure (669)
  unsure (673)
  unsure (680)
  unsure (688)
ggatgtacttaaccacatccagcttggaggatctgttgcaggcacggancctgagggcagtggcaaggccaagaacangccattcatgacaaagacggcctacttctactgggtgaccagggaanaggggtcctttnaatggttccgaggggtcatgaatgangtggctgagaaggacaaggatggagtcnttnaactccacaaccactgctcgagtgtgcaccangaaggggatgttcgttctgccctcattgtcatgctccacgagctccagcacgcgaagaaaggagtcgacntcttgtctggaactagtgtcaacacgcattttgcacggccgaattggcgaatcctcttccaacatgttgcactgaaccacganaaccnncgcntccganttttctactgtggtgaccccntccttgtnccanagctaccgcacctgtcancggactccccctaanacaaatactacnttccagttccacacaggacaacttctnnttggaagaactggaaaaaaanctctncgcgtnncctctnatggtttcncaatntgtcaattncnattgttnttaccctntttccnaanttcannaatcccacganggganttngttttcgcnnttnnannaaaaaaaaanacgggccgcccgctctcaaaggatcnancctncctttcnccttcctn

SEQ ID NO: 2 - protein, 134 aa, Zea mays
  UNSURE (10)
  UNSURE (26)
  UNSURE (30)
  UNSURE (38)
  UNSURE (48)..(49)
  UNSURE (59)
  UNSURE (83)
  UNSURE (110)
  UNSURE (112)
  UNSURE (114)
  UNSURE (116)
  UNSURE (123)
  UNSURE (127)
  UNSURE (133)
PEGSGKAKNXPFMTKTAYFYWVTREXGSFXWFRGVMNXVAEKDKDGVXXLHNHCSSVHXEGDVRSALIVMLHELQHAKKGVDXLSGTSVNTHFARPNWRILFQHVALNHXNXRXRXFYCGDPXLVPXLPHLSXD

SEQ ID NO: 3 - DNA, 562 bp, Oryza sativa
  unsure (535)
  unsure (545)
gtttaaacgcggcgcgcctacttctactgggtgacgcgggagcaggggtcgttcgagtggttccggggggtgatggacgaggtggcggagacggacaagaagggggtgatcgagctgcacaactactgcaccagcgtgtacgaggaaggggacgcccggtcggcgctcatcgctatgctccagtcgctcaaccacgccaagcacggcgtcgacgtcgtctccggcacccgcgtcaagacccacttcgcccgccccaactggcgcaacgtctacaagcgcatcgccctcaaccaccgcgaccaacgcgtcggggtgttctactgtggcgcgccggtgctgacgaaggaactgcgtgagtcgctcaagatttctcgagaaagacgagcacgaaattcgacttccacaaggagaatttctagttatctggaatcaaaaccaaagttttcgcacggccatgtttagtacatacaaagttctatacatatgacaagtatgatgacatacatattggaatgtagagggattagatcaaagnaggtattgcntgattgtgggccaggcg

SEQ ID NO: 4 - protein, 116 aa, Oryza sativa
TRRAYFYWVTREQGSFEWFRGVMDEVAETDKKGVIELHNYCTSVYEEGDARSALIAMLQSLNHAKHGVDVVSGTRVKTHFARPNWRNVYKRIALNHRDQRVGVFYCGAPVLTKELR

SEQ ID NO: 5 - DNA, 489 bp, Triticum aestivum
  unsure (234)
  unsure (310)
  unsure (405)
  unsure (417)..(418)
  unsure (424)
  unsure (433)
  unsure (454)
  unsure (464)..(465)
  unsure (476)
  unsure (489)
cttcgagtggttccgcggcgtgatggacgaggtggcggagacggacaggaagggcgtcatcgagctgcacaactactgcaccagcgtgtacgaggaaggggacgcccggtccgcgctcatcgccatgctccagtcgctcaaccacgccaagcacggcgtcgacgtggtgtccggcacccgcgtcaagacccacttcgccaggcctaactggcgcaacgtctacaagcgcatcgntcaaccaccagaaccagcgcgtcggagtgttctactgcggcgccccggtgctgaccaaggagctgcgcaactggcnaggacttctcgcggaagaccaacaccaagttcgagttccacaaggagaactttagtctctctctctctatatatatgtaaggtatgaatggttanaaagggatcgannaccaantaggatttncttaacaattggttgggggtnaaacccaaannttttttttaanatatatatctttn

SEQ ID NO: 6 - protein, 122 aa, Triticum aestivum
  UNSURE (78)..(79)
  UNSURE (103)..(104)..(105)
FEWFRGVMDEVAETDRKGVIELHNYCTSVYEEGDARSALIAMLQSLNHAKHGVDVVSGTRVKTHFARPNWRNVYKRIXXNHQNQRVGVFYCGAPVLTKELRNXXXDFSRKTNTKFEFHKENF

SEQ ID NO: 7 - DNA, 648 bp, Zea mays
ccacgcgtccgggatgtacttaaccacatccagcttggaggatctgttgcaggcacggagcctgagggcagtggcaaggccaagaagaggccattcatgacaaagagggcctacttctactgggtgaccagggaagaggggtcctttgaatggttccgaggggtcatgaatgaggtggctgagaaggacaaggatggagtcattgaactccacaaccactgctcgagtgtgcaccaggaaggggatgtacgttctgccctcattgtcatgctccaggagctccagcacgcgaagaagggagtcgacatcttgtctggaactagtgtcaagacgcattttgcacggccgaattggcgaagcgtcttcaaacatgttgcagtgaaccacgagaaccaacgcatcggagttttctactgtggtgagcccgtccttgtgccacagctacggcagctgtcagcggacttcacccacaagacaaatactaagttcgagttccacaaggagaacttctaatggaggaactggagaaaagctctaggcgtggccctctaatggtttcagaatatgtgaattacgatggttgtaacatatatacgagtagaagaataagacgatgggagttgttttgttgtaaaaaaaaaaaaaaag

SEQ ID NO: 8 - protein, 170 aa, Zea mays
PRVRDVLNHIQLGGSVAGTEPEGSGKAKKRPFMTKRAYFYWVTREEGSFEWFRGVMNEVAEKDKDGVIELHNHCSSVHQEGDVRSALIVMLQELQHAKKGVDILSGTSVKTHFARPNWRSVFKHVAVNHENQRIGVFYCGEPVLVPQLRQLSADFTHKTNTKFEFHKENF

SEQ ID NO: 9 - DNA, 1200 bp, Oryza sativa
cacgaggtttaaacgcggcgcgcctacttctactgggtgacgcgggagcaggggtcgttcgagtggttccggggggtgatggacgaggtggcggagacggacaagaagggggtgatcgagctgcacaactactgcaccagcgtgtacgaggaaggggacgcccggtcggcgctcatcgctatgctccagtcgctcaaccacgccaagcacggcgtcgacgtcgtctccggcacccgcgtcaagacccacttcgcccgccccaactggcgcaacgtctacaagcgcatcgccctcaaccaccgcgaccaacgcgtcggggtgttctactgtggcgcgccggtgctgacgaaggaactgcgtgagctcgctcaagatttctcgagaaagacgagcacgaaattcgacttccacaaggagaatttctagttatctggaatcaaaaccaaagttttcgcacggccatgtttagtacatacaaagttctatacatatgacaagtatgatgacatacatattggaaatgtagagggattagatcaaagtaggtattgcttgattgtggccaggcttggccagataatttcatcggttttttgctctggaagaataatccaatgcccccctttgtacagatcttctcccagataatactttgtaatacttagagtagccaatttgataaaatcagtttgtatctagtaacatgtagagagtttcatggaggcctaatcaggtcaaaaatatcacaaatgtttggccaagaacaagaaaaatcatgcccattgcaagaaagaaattgcactttttgtggctggtactgaattgcggacctgaagaacacacctgatattggagcactgcacatggtggtgaccaacaaacaagaacaacctcatgtcctatgtccacaaccaatgagcactgccaacatattagggttctagaagatgatgagcatgatggtaccctcctggcccacatagcaactagagtagttgtctttggtttttactttttggaatatctgttctttctgttccttcaattttattccattgtattctgtgaagtgcatgataaagaggggattacgcaaaataatactttttgcaattcatggctgtaaatgcactgttgaacaataaaaatctgagctttttttttttaaaaaaaaaaaaaaaaaaaaaaaaaaaaaaaaaa

SEQ ID NO: 10 - protein, 137 aa, Oryza sativa
TRRAYFYWVTREQGSFEWFRGVMDEVAETDKKGVIELHNYCTSVYEEGDARSALIAMLQSLNHAKHGVDVVSGTRVKTHFARPNWRNVYKRIALNHRDQRVGVFYCGAPVLTKELRELAQDFSRKTSTKFDFHKENF

SEQ ID NO: 11 - DNA, 543 bp, Triticum aestivum
gcacgagcttcgagtggttccgcggcgtgatggacgaggtggcggagacggacaggaagggcgtcatcgagctgcacaactactgcaccagcgtgtacgaggaaggggacgcccggtccgcgctcatcgccatgctccagtcgctcaaccacgccaagcacggcgtcgacgtggtgtccggcacccgcgtcaagacccacttcgccaggcctaactggcgcaacgtctacaagcgcatcgcgctcaaccaccagaaccagcgcgtcggagtgttctactgcggcgccccggtgctgaccaaggagctgcgcgagctggcgcaggacttctcgcggaagaccaacaccaagttcgagttccacaaggagaacttctagctctctctctctctatatatatgtaaggatatggatgggtgtagagaggggatcggagtagcgcaaagtaggtatttgcttgaccagttgggttgggtgggtcaaaaccccaacagtttttttttttaattattattattattcttcttaaaaaaaaaaaaaaaaa

SEQ ID NO: 12 - protein, 124 aa, Triticum aestivum
TSFEWFRGVMDEVAETDRKGVIELHNYCTSVYEEGDARSALIAMLQSLNHAKHGVDVVSGTRVKTHFARPNWRNVYKRIALNHQNQRVGVFYCGAPVLTKELRELAQDFSRKTNTKFEFHKENF

SEQ ID NO: 13 - DNA, 647 bp, Zea mays
gtgtggtgaagctgccatcgccgctacaccaccttgccggtttcaatgccttctggtacgctcaccacctgctggtccttgcgtatgtcctgctggtggtgcactcctacttcatattcctcaccagggagtggtacaagaagacgacatggatgtacctgattgtccctgtcctcttctatgcctgtgaaagagtcatcaggaaatttcgtgagaacaactaccatgcgggaattgtgagggcagcaatttatccgggagatgtgctctctattcacatgaagaagccacagggtttcaagtacaagagtgggatgtatctgtttgttaaatgcccagaagtctcgcccttcgagtggcaccccttctctataacttcggcaccaggcgatgactacttgagtgtgcatatccgtacgctgggtgactggacatccgaactgcggatgctttttgggaaggcttgccaggcacaagttaacttccaagaaaggctacccttacaagacttgaaactacagttgtggcagacgcccagacagaggacactaggtttccccaaggtctacatagacgggccatacggtgcaccagcacaaaattacaggaaatatgacattcttctgcttattggccttggaatagga

SEQ ID NO: 14 - protein, 167 aa, Zea mays
LPSPLHHLAGFNAFWYAHHLLVLAYVLLVVHSYFIFLTREWYKKTTWMYLIVPVLFYACERVIRKFRENNYHAGIVRAAIYPGDVLSIHMKKPQGFKYKSGMYLFVKCPEVSPFEWHPFSITSAPGDDYLSVHIRTLGDWTSELRMLFGKACQAQVNFQERLPLQDL

SEQ ID NO: 15 - DNA, 577 bp, Oryza sativa
  unsure (340)
  unsure (427)
  unsure (496)..(497)
  unsure (507)
  unsure (517)
  unsure (519)..(520)
  unsure (533)
  unsure (537)
  unsure (541)
  unsure (549)
  unsure (552)
  unsure (568)
gttgggattcagaaggttgcagtgtatcccgggaatgtattggctctttatatgtcgaagccacctggtttcagataccgtagtgggcagtacatcttcataaaatgcactgctgtgtctccatatgaatggcatccattttccataacatcagcacctggagatgattatcttagtgttcatattcgcacaaggggtgattgggcttcacggcttaagaactgttttctcctgaggcatgccgaccccccaactgagggagaaaatgggactacttagactgacctttcccaaagggaataacggacgaaaacccaagatccccaaaacttttgggccnatggaccgttatggtgcaccggcacaagattaccgtgaaatacgatgtgctacttctcaaccgggctgggaaccggaccaccctttngattacattgtgaagactgctaacacatcaaggtgaggataattggacacggaccggagacacacaagnnaagaagaacnttatgacaanannccactcacggggnacanaagngcctttantgntaagggtataacagtntcaaagcag

SEQ ID NO: 16 - protein, 76 aa, Oryza sativa
VGIQKVAVYPGNVLALYMSKPPGFRYRSGQYIFIKCTAVSPYEWHPFSITSAPGDDYLSVHIRTRGDWASRLKNCF

SEQ ID NO: 17 - DNA, 457 bp, Glycine max
  unsure (336)
gcatttccaagttgtataactcaatgtatccaaggttgtccggatccagttcctccatgataagggcagcatattcctctgcacgatcttttaattttgacagcttattggctgaagcgcttaaggtgataatctcttttacttcttcttcattaattcgtccatcggcatctttgtccaccatgtcaaaaaaggtctgaagccgtgaatcaaaactctggtctgtaatttgctcccaaaattcacgcaactgatccttcgttatggaagctgatgttatccctcgacgacgagctaatgcatcgaataactcaccggcaaactccttcgattcgntcatccctatgcactggctaaaagcgaagtcttggggagctttgcatcaatggccaactcatccgaagcgcttctcaacctgtgaccaacctttagtggctgctttgggcaagaacttaag

SEQ ID NO: 18 - protein, 144 aa, Glycine max
  UNSURE (26)
  UNSURE (34)
AATKGWSQVEKRFDELAIDAKLPKTXFSQCIGMXESKEFAGELFDALARRRGITSASITKDQLREFWEQITDQSFDSRLQTFFDMVDKDADGRINEEEVKEIITLSASANKLSKLKDRAEEYAALIMEELDPDNLGYIELYNLE

SEQ ID NO: 19 - DNA, 577 bp, Triticum aestivum
  unsure (214)
  unsure (228)
  unsure (282)
  unsure (370)
  unsure (372)
  unsure (412)
  unsure (429)
  unsure (450)
  unsure (462)
  unsure (476)
  unsure (482)
  unsure (488)
  unsure (510)
  unsure (514)
  unsure (519)
  unsure (526)
  unsure (538)
  unsure (542)
  unsure (545)
gaatacgatgtgctcctgctcattggactgggcattggagccactccattgattagcattgtgaaggatgtgcttaaccacacccagcatggaggatctgtttcaggcacggagcctgagggcagtggcaaggccaagaagaggccattcatgacgaagagggcctacttctactgggtgaccagagaagagggatctttcgaatggttccgangggtcatgaacgangtgggctgagaaaggacaaggatgggagtcattgaactccacaacaactgctcnattgtgtacaaggaagggatgcacgttctgcactccattgtcatgctccaagactccaacaatgcaaagaaaggggtcgacatcttgncnggaactaatgtcaagacgcacttcgcgcgtcccattggcnaacgtctccaacatgtngcatgaaccatgagaacaacnctttgggtttcnacggggtaacccgncttttncaaagntcggaatggcacaaattccccnaagncaacnaatttgntccaagggaatncaanganacggaaaccgggcgggcccaaggttaaatgat

SEQ ID NO: 20 - protein, 134 aa, Triticum aestivum
  UNSURE (71)
  UNSURE (75)
  UNSURE (78)
  UNSURE (123)
YDVLLLIGLGIGATPLISIVKDVLNHTQHGGSVSGTEPEGSGKAKKRPFMTKRAYFYWVTREEGSFEWFRXVMNXVGXERTRMGVIELHNNCSIVYKEGMHVLHSIVMLQDSNNAKKGVDILXGTNVKTHFARP

SEQ ID NO: 21 - DNA, 1257 bp, Oryza sativa
gcacgaggttgggattcagaaggttgcagtgtatcccgggaatgtattggctctttatatgtcgaagccacctggtttcagataccgtagtgggcagtacatcttcataaaatgcactgctgtgtctccatatgaatggcatccattttccataacatcagcacctggagatgattatcttagtgttcatattcgcacaaggggtgattggacttcacggcttagaactgttttctctgaggcatgccgaccccccactgagggagaaagtggactacttagagctgacctttccaagggaataacggacgaaaacgcaagattcccaaaacttttggtcgatggaccgtatggtgcaccggcacaagattaccgtgaatacgatgtgctacttctcatcgggctgggcatcggagccacccctttgattagcattgtgaaggacgtgcttaaccacattcaaggtgagggatcagttggaaccacggagccggagagcagcagcaaggcgaagaagaaacctttcatgacgaagagagcctacttctactgggtgacgagagaggagggctcgtttgagtggttcagaggcgtcatgaacgaggtgtctgagaaggacaaggatggagtcattgagctccataaccactgctcaagcgtgtaccaggaaggcgatgctcgttctgctctcattgtcatgctccaagaacttcagcatgcgaagaagggcgtcgatatcttgtcgggaactagtgtgaagacccatttcgcacgacctaattggcgaagcgtcttcaagaaggttgcggtcaaccatgagaaccagcgcgtcggtgtgttctactgtggtgagcctgtgctggttccccaactaaggcagttgtcagcagatttcacccacaagacaaacacaagatttgatttccacaaggagaacttctaatggtacaaattgagaaatacccgtgtatggttttgtatgtagttctttatcatgtgaattatatggtttctaatatatataaagttggacaaaataaatgaaatgatggaagctattttgttttttaagatgtcaaaagtctgcaatatctttttacaagagtgctgtctattcatgtatacacctagtggaagaagctgtgcttcatgttgtagcttacatagatgaagggaagttctctttgttgtgaccaaaggatgcctagaatcatgtaacattgtgatgttccctttcaaaaaaaaaaaaaaaaaaaa

SEQ ID NO: 22 - protein, 313 aa, Oryza sativa
HEVGIQKVAVYPGNVLALYMSKPPGFRYRSGQYIFIKCTAVSPYEWHPFSITSAPGDDYLSVHIRTRGDWTSRLRTVFSEACRPPTEGESGLLRADLSKGITDENARFPKLLVDGPYGAPAQDYREYDVLLLIGLGIGATPLISIVKDVLNHIQGEGSVGTTEPESSSKAKKKPFMTKRAYFYWVTREEGSFEWFRGVMNEVSEKDKDGVIELHNHCSSVYQEGDARSALIVMLQELQHAKKGVDILSGTSVKTHFARPNWRSVFKKVAVNHENQRVGVFYCGEPVLVPQLRQLSADFTHKTNTRFDFHKENF

SEQ ID NO: 23 - DNA, 918 bp, Glycine max
tcgatcataaattaattacaaaaataacataggttgtcgcaacatggagattcacgaaaaccaacacgagtcatggtcggaaacggagagcacgggaagccggagcaggagagtgggcttcagtgggcctctgagcggaccactgagcgggcctttgagtgggcctttggtttcttctaacaaaagaaacagcagcaagaacaaaagtgcgaggttcaaggacgacgaggagatggtggagatcacgctggacgtccgcgacgacgccgtttcggtccagaacatccgcggcggcgactccgagacggcgttcctcgccagccgcctcgagatgaggccgtcgtcgttttccgatcggctgagacaggtgtcgcgggaactgaagcgcatgacatcaaacaaggccttcgatagggttgaccgcagcaaatccggtgctgcgcgcgcccttggtggtcttaagttcatgaccaaagcaggcactgaaggttggtcacaggttgagaagcgcttcgatgagttggccattgatgcaaagctccccaagactcgctttagccagtgcatagggatgaacgaatcgaaggagtttgccggtgagttattcgatgcattagctcgtcgtcgagggataacatcagcttccataacgaaggatcagttgcgtgaattttgggagcaaattacagaccagagttttgattcacggcttcagaccttttttgacatggtggacaaagatgccgatggacgaattaatgaagaagaagtaaaagagattatcaccttaagcgcttcagccaataagctgtcaaaattaaaagatcgtgcagaggaatatgctgcccttatcatggaggaactggatccggacaaccttggatacattgagttatacaacttggaaatgcctcgtgc

SEQ ID NO: 24 - protein, 291 aa, Glycine max
MEIHENQHESWSETESTGSRSRRVGFSGPLSGPLSGPLSGPLVSSNKRNSSKNKSARFKDDEEMVEITLDVRDDAVSVQNIRGGDSETAFLASRLEMRPSSFSDRLRQVSRELKRMTSNKAFDRVDRSKSGAARALGGLKFMTKAGTEGWSQVEKRFDELAIDAKLPKTRFSQCIGMNESKEFAGELFDALARRRGITSASITKDQLREFWEQITDQSFDSRLQTFFDMVDKDADGRINEEEVKEIITLSASANKLSKLKDRAEEYAALIMEELDPDNLGYIELYNLEMPR

SEQ ID NO: 25 - DNA, 876 bp, Triticum aestivum
gcacgaggaatacgatgtgctcctgctcattggactgggcattggagccactccattgattagcattgtgaaggatgtgcttaaccacacccagcatggaggatctgtttcaggcacggagcctgagggcagtggcaaggccaagaagaggccattcatgacgaagagggcctacttctactgggtgaccagagaagagggatctttcgaatggttccgaggggtcatgaacgaggtggctgagaaggacaaggatggagtcattgaactccacaaccactgctcgagtgtgtacgaggaaggggatgcacgttctgcactcattgtcatgctccaagagctccagcatgcgaagaaaggggtcgacatcttgtctggaactagtgtcaagacgcacttcgcgcgtcccaattggcgaagcgtcttcaaacatgttgcagtgaaccatgagaaccaacgcgttggagttttctactgcggtgagcctgtccttgtgccacagctacggcagtggtcagcagacttcacccacaagacgaacacaaagtttgagttccacaaggagaacttctaatggcataactggagaagctctggacgtggtcctctaatgatttcagaatgtgaattacgagggttgtaatatatatacgagtagaataaaatgatgggagttattttgttgtagatgccaaaagtcaaccatgttctttttgcaatgtgctctgtccatccctgtttactcctaccaggagatatactctgtcgtctaggcgaagtagataaagtgtctcttgcatacaagcaacattagaagttgtaacacatgcgaacatgtcatttcgtttttagaccatttgatagtcaaaaaaaaaa

SEQ ID NO: 26 - protein, 190 aa, Triticum aestivum
HEEYDVLLLIGLGIGATPLISIVKDVLNHTQHGGSVSGTEPEGSGKAKKRPFMTKRAYFYWVTREEGSFEWFRGVMNEVAEKDKDGVIELHNHCSSVYEEGDARSALIVMLQELQHAKKGVDILSGTSVKTHFARPNWRSVFKHVAVNHENQRVGVFYCGEPVLVPQLRQWSADFTHKTNTKFEFHKENF

SEQ ID NO: 27 - DNA, 553 bp, Oryza sativa
  unsure (391)
  unsure (442)
  unsure (453)
  unsure (519)
  unsure (552)
gtttaaacccctttgattagcattgtgaaggacgtgcttaaccacattcaaggtgagggatcagttggaaccacggagccggagagcagcagcaaggcgaagaagaaacctttcatgacgaagagagcctacttctactgggtgacgagagaggagggctcgtttgagtggttcagaggcgtcatgaacgaggtgtctgagaaggacaaggatggagtcattgagctccataaccactgctcaagcgtgtaccaggaaggcgatgctcgttctgctctcattgtcatgctccaagaacttcagcatgcgaagaagggcgtcgatatcttgtcgggaactagtgtgaagacccatttcgcacgacctaattggcgaagcgtcttcaaagaangttgcggtcaaccatgagaaccagcgcgtccgtgtgttctactgtggtgancctgtgctggntcccaactaaggcatttgtcagcagatttcacccacaagacaaacacaagatttgatttccacaanggagaacttctaatggtacaaatttgagaaatnc

SEQ ID NO: 28 - protein, 174 aa, Oryza sativa
  UNSURE (132)
  UNSURE (149)
  UNSURE (153)..(154)
TPLISIVKDVLNHIQGEGSVGTTEPESSSKAKKKAKKKPFMTKRAYFYWVTREEGSFEWFRGVMNEVSEKDKDGVIELHNHCSSVYQEGDARSALIVMLQELQHAKKGVDILSGTSVKTHFARPNWRSVFKXVAVNHENQRVRVFYCGXPVLXXQLRHLSADFTHKTNTRFDFH

SEQ ID NO: 29 - DNA, 685 bp, Oryza sativa
ggcacgaggtttaaacccctttgattagcattgtgaaggacgtgcttaaccacattcaaggtgagggatcagttggaaccacggagccggagagcagcagcaaggcgaagaagaaacctttcatgacgaagagagcctacttctactgggtgacgagagaggagggctcgtttgagtggttcagaggcgtcatgaacgaggtgtctgagaaggacaaggatggagtcattgagctccataaccactgctcaagcgtgtaccaggaaggcgatgctcgttctgctctcattgtcatgctccaagaacttcagcatgcgaagaagggcgtcgatatcttgtcgggaactagtgtgaagacccatttcgcacgacctaattggcgaagcgtcttcaagaaggttgcggtcaaccatgagaaccagcgcgtcggtgtgttctactgtggtgagcctgtgctggttccccaactaaggcagttgtcagcagatttcacccacaagacaaacacaagatttgatttccacaaggagaacttctaatggtacaaattgagaaatacccgtgtatggttttgtatgtagttctttatcatgtgaattatatggtttctaatatatataaagttggacaaaataaatgaaatgatggaagctattttgttttttaaaaaaaaaaaaaaaaaaaa

SEQ ID NO: 30 - protein, 174 aa, Oryza sativa
TPLISIVKDVLNHIQGEGSVGTTEPESSSKAKKKPFMTKRAYFYWVTREEGSFEWFRGVMNEVSEKDKDGVIELHNHCSSVYQEGDARSALIVMLQELQHAKKGVDILSGTSVKTHFARPNWRSVFKKVAVNHENQRVGVFYCGEPVLVPQLRQLSADFTHKTNTRFDFHKENF

SEQ ID NO: 31 - DNA, 734 bp, Zea mays
  unsure (9)
  unsure (503)
  unsure (612)
  unsure (634)
  unsure (678)
  unsure (697)
  unsure (717)
gatcaacancgcatagctcgaaacttagcaaagctcttagcatgaagcttgcatcaaacaaggacacaggtccattttaccactactggcaagagttcatgtacttccttgaggagaactggaagcgcatttgggttatgactctctggctctcaatctgcattggcctctttatttggaagttcatccaataccgtaatcgagcagtatttcacatcatgggttattgtgtgaccactgcaaaaggtgctgcagagactctcaaattcaatatggccctggttctttttcctgtttgccgaaatacaatcacttggattcgatcgaagacaaagatcggagctgttgtacccttcaatgataacataaacttccataaggtaatagcagcaggtgttgcagttggtgttgctttgcatgcaggtgctcacctgacatgtgattttcctcggctgctccatgcaagtgatgctgcctatgaaccaatgaagcctttctttggngacaaaaggccaccaaattactggtggtttgtaaagggaactgaagggtggacaggtgtggtcatggatgtacttaagactatagccttcgtattggcccaaccatggnttcggcgtaataagctcaaggnttctaatcccctcaagaaaatgactggcttcaatgccttttggnttacgcaccacttaattgntaatgtgtatgcactggccnttgtccaaggggatttg

SEQ ID NO: 32 - protein, 234 aa, Zea mays
  UNSURE (196)
  UNSURE (203)
  UNSURE (218)
  UNSURE (224)
  UNSURE (231)
LSKALSMKLASNKDTGPFYHYWQEFMYFLEENWKRIWVMTLWLSICIGLFIWKFIQYRNRAVFHIMGYCVTTAKGAAETLKFNMALVLFPVCRNTITWIRSKTKIGAVVPFNDNINFHKVIAAGVAVGVALHAGAHLTCDFPRLLHASDAAYEPMKPFFGDRPPNYWWFVKGTEGWTGVVMDVLKTIAFVLAQPWXRRNKLKXSNPLKKMTGFNAFWXTHHLIXNVYALAXVQG

SEQ ID NO: 33 - DNA, 600 bp, Oryza sativa
  unsure (399)
  unsure (415)
  unsure (440)
  unsure (453)
  unsure (467)
  unsure (473)
  unsure (484)
  unsure (486)
  unsure (490)
  unsure (494)
  unsure (499)
  unsure (509)
  unsure (544)
  unsure (548)
  unsure (554)..(555)
  unsure (558)
  unsure (567)
  unsure (576)
  unsure (581)
  unsure (585)
  unsure (594)
gtggttcgtgaaggggacggaggggtggacggggctggtgatgctggtgctcatggcggtggcgttcaccctcgccacgccgtggttccgccgcggccgcctccgcctcccccgcccgctcaaccgcctcacggggttcaacgccttctggtactcccaccactgcttcgtcatcgtctacgccctcctcatcgtccacggctactacctcttccttaccaaggattggtacaagaaaacgacgtggatgtacctggcggtgccgatgttcctgtacgcgtgcgagaggctgacgagggcgctccggtcgagcgtgaggccagtgaagatatcaagttgcggtgtaccccggaaatgtgtgtcgctgacttctccaagccaagggtttcaagacaagantggcagacatttcgtnactgtgcgccgtctgccgttcatgnaccattctcatangtggccacagacgntagtangtcaatagagntngtantganaggantaagaagttntaaggtttcggcacgacgaagaaaacggtttcggngagnaaacgnnggnatacaacangtccaagtntatanggcnaggaacggnagaaaa

SEQ ID NO: 34 - protein, 120 aa, Oryza sativa
WFVKGTEGWTGLVMLVLMAVAFTLATPWFRRGRLRLPRPLNRLTGFNAFWYSHHCFVIVYALLIVHGYYLFLTKDWYKKTTWMYLAVPMFLYACERLTRALRSSVRPVKISQVAVYPGNV

SEQ ID NO: 35 - DNA, 549 bp, Glycine max
  unsure (467)
  unsure (520)
  unsure (534)
  unsure (549)
aacaaactgtcaaacatccagaagcaagcagaggaatatgcggctttgattatggaagaattggaccctgatgacacaggatacatcatgatagacaacctggagacgctcttgttgcatggaccagaggaaactacaagaggagaaagtaagtacctgagccaaatgctaagtcaaaaacttaagcctacatttgcggacagtgcagttatgaggtggtgtagagatgccaagtacttcttgctggacaactggcaaagatcttgggtacttgcactttggattggtgtgatgtttggcctatttgcctataaatttgtgcaatataggagaaaagctgcctatgaggtgatgggccattgtgtatgcatggcaaaaggtgcagccgagacacttaaattgaacatggtcttatcttgttacctgtttgccgcaacacccatcacctggctcaggaataagaccangctaggtgtgtagttcctttggatgacaacatcaacttccacaaggtatagcngtggcatagcagtngctgttgctgtgaan

SEQ ID NO: 36 - protein, 159 aa, Glycine max
  UNSURE (48)
  UNSURE (157)
NKLSNIQKQAEEYAALIMEELDPDDTGYIMIDNLETLLLHGPEETTRXGESKYLSQMLSQKLKPTFADSAVMRWCRDAKYFLLDNWQRSWVLALWIGVMFGLFAYKFVQYRRKAAYEVMGHCVCMAKGAAETLKLNMVLSCYLFAATPITWLRNKTXLG

SEQ ID NO: 37 - DNA, 483 bp, Triticum aestivum
  unsure (2)
  unsure (10)
  unsure (77)
  unsure (181)
  unsure (245)
  unsure (265)
  unsure (273)
  unsure (285)
  unsure (324)
  unsure (327)
  unsure (366)
  unsure (369)
  unsure (373)
  unsure (385)
  unsure (392)
  unsure (402)
  unsure (438)
  unsure (445)
  unsure (447)
  unsure (457)
  unsure (462)
  unsure (464)
  unsure (481)
  unsure (483)
gntgagccancacctccggccgacgggggagcccaacccgctccggcgctggtaccgccgcgccagctacttcctcnaggacaactggcggcgctgctgggtgatcctgctgtggctctccatctgcgtgggcctcttcacgtggaagttcatgcagtaccgcgagcgcgccgtgttcaangtgatgggctactgcgtgtgcgtgggcaaaggcggcgccgagacgctcaagttcaacatggggntcatcctgctccccgtgtgncgcaaaancaacaacgtggntccgcaaacgcaccgccgcgggccggttcgtggcgttcnacnacaacatcaacttccacaaagggattccccgcggggatntcngtcngggcgggggtgnaaaacanctcccaattntcgtgcgaatttccgcgcctgctggacgccaacgangggaggngnaagaagccangaaancngttctttcggggattancn

SEQ ID NO: 38 - protein, 143 aa, Triticum aestivum
  UNSURE (2)
  UNSURE (25)
  UNSURE (59)
  UNSURE (81)
  UNSURE (87)
  UNSURE (90)
  UNSURE (94)
  UNSURE (107)..(108)
  UNSURE (120)
  UNSURE (123)
  UNSURE (127)
  UNSURE (129)
  UNSURE (132)
SXHLRPTGEPNPLRRWYRRASYFLXDNWRRCWVILLWLSICVGLFTWKFMQYRERAVFXVMGYCVCVGKGGAETLKFNMGXILLPCXAKXTTWXRKRTAAGRFVAFXXNINFHKGFPAGXSVXAGVXNXSQXSCEFPRLLDAN

SEQ ID NO: 39 - DNA, 1631 bp, Oryza sativa
gcacgaggtggttcgtgaaggggacggaggggtggacggggctggtgatgctggtgctcatggcggtggcgttcaccctcgccacgccgtggttccgccgcggccgcctccgcctcccccgcccgctcaaccgcctcacggggttcaacgccttctggtactcccaccactgcttcgtcatcgtctacgccctcctcatcgtccacggctactacctcttccttaccaaggattggtacaagaaaacgacgtggatgtacctggcggtgccgatgttcctgtacgcgtgcgagaggctgacgagggcgctccggtcgagcgtgaggccagtgaagatactcaaggttgcggtgtaccccggaaatgtgctgtcgctgcacttctccaagccacagggtttcaagtacaagagtgggcagtacatcttcgtcaactgtgccgccgtctcgccgttccaatggcacccattctccatcacgtcggccccacaggacgactacgtcagcgtccacatcaggacgctcggtgactggacacgggagcttaagaacgtcttctcaagggtctgccggccaccgacggaagggaagagcgggttgctccgggcggagtacgaccgcgacggcgccatgaccaacccaagcttccccaaggtgcttatcgacgggccgtacggcgcaccggcgcaggactacaagcagtacgacatcgtcctcctcgtcggcctcgggatcggcgccaccccgatgatctccatcatcaaggacatcataaacaacatgaggcagctggacggcgacctcgaggacggcgacggcaacgataactcggtgtcgtcgtcgtcggcggcgttcaagacgcggcgcgcctacttctactgggtgacgcgggagcaggggtcgttcgagtggttccggggggtgatggacgaggtggcggagacggacaagaagggggtgatcgagctgcacaactactgcaccagcgtgtacgaggaaggggacgcccggtcggcgctcatcgctatgctccagtcgctcaaccacgccaagcacggcgtcgacgtcgtctccggcacccgcgtcaagacccacttcgcccgccccaactggcgcaacgtctacaagcgcatcgccctcaaccaccgcgaccaacgcgtcggggtgttctactgtggcgcgccggtgctgacgaaggaactgcgtgagctcgctcaagatttctcgagaaagacgagcacgaaattcgacttccacaaggagaatttctagttatctggaatcaaaaccaaagttttcgcacggccatgtttagtacatacaaagttctatacatatgacaagtatgatgacatacatattggaaatgtagagggattagatcaaagtaggtattgcttgattgtggccaggcttggccagataatttcatcggttttttgctctggaagaataatccaatgcccccctttgtacagatcttctcccagataatactttgtaatacttagagtagccaatttgataaaatcagtttgtatctagtaacatgtagagagtttcatggaggcctaatcaggtcaaaaatatcacaaatgtttggccaagaacaagaaaaaaaaaaaaaaaaaa

SEQ ID NO: 40 - protein, 422 aa, Oryza sativa
TRWFVKGTEGWTGLVMLVLMAVAFTLATPWFRRGRLRLPRPLNRLTGFNAFWYSHHCFVIVYALLIVHGYYLFLTKDWYKKTTWMYLAVPMFLYACERLTRALRSSVRPVKILKVAVYPGNVLSLHFSKPQGFKYKSGQYIFVNCAAVSPFQWHPFSITSAPQDDYVSVHIRTLGDWTRELKNVFSRVCRPPTEGKSGLLRAEYDRDGAMTNPSFPKVLIDGPYGAPAQDYKQYDIVLLVGLGIGATPMISIIKDIINNMRQLDGDLEDGDGNDNSVSSSSAAFKTRRAYFYWVTREQGSFEWFRGVMDEVAETDKKGVIELHNYCTSVYEEGDARSALIAMLQSLNHAKHGVDVVSGTRVKTHFARPNWRNVYKRIALNHRDQRVGVFYCGAPVLTKELRELAQDFSRKTSTKFDFHKENF

SEQ ID NO: 41 - DNA, 2365 bp, Glycine max
gcacgagaacaaactgtcaaacatccagaagcaagcagaggaatatgcggctttgattatggaagaattggaccctgatgacacaggatacatcatgatagacaacctggagacgctcttgttgcatggaccagaggaaactacaagaggagaaagtaagtacctgagccaaatgctaagtcaaaaacttaagcctacatttgcggacagtgcagttatgaggtggtgtagagatgccaagtacttcttgctggacaactggcaaagatcttgggtacttgcactttggattggtgtgatgtttggcctatttgcctataaatttgtgcaatataggagaaaagctgcctatgaggtgatgggccattgtgtatgcatggcaaaaggtgcagccgagacacttaaattgaacatggctcttatcttgttacctgtttgccgcaacaccatcacctggctcaggaataagaccaagctaggtgttgtagttcctttggatgacaacatcaacttccacaaggtaatagctgtggcaatagcagttgctgttgctgtacattccatctatcatcttacttgtgattttcctcgccttcttcatgcaagcgatgaaaagtacaagctcatgcaaccttttttcggagacagaccatcagattattggtattttgtcaaatcatgggaaggagtaacagggattataatagttgtgctaatggcaatagcctttacgctggctaatcctaggttcaggagaggccgggccaaactacccaaacctttcaacaaattcacgggtttcaatgccttttggtattcccatcacctcttcgtcattgtctatgccctgttggttgtacatggaatcaaactttacttgactaaggaatggtacaagaaaacgacctggatgtatctggctattcccatcaccatttatgcattggaaagactggttagagcattcagatcgagcattaagtccgtcaaaatattgaaggtgactctttatcctggaaacgtgttatcacttaaaatgtcaaagccgcaggggtttagctacaaaagtggacaatacatgtttgtgaattgtgctgctgtgtctccatttgaatggcatccattttccataacttccgcccctgatgatgattaccttagcgttcacataaaaatacttggtgactggactcgaagtctgaaagccaaattcacacaggcgtgccagcaacccctaaatggacagagtggacttctaagagctgaatgcttgaaaggagataacagcccaagttcctttcctaaggttctggtggatggtccgtatggggcgccagcacaagactacagggagtatgaggtggtgttgctggtggggcttggaattggggctacaccaatgataagtatactaaaggacatggtgaataattttaaggcgaatgatgaggaggagggagggcaagagagggtgagtgacttcaagacaaggagggcatacttctactgggtgactacacatcacggttcatttgactggttcaaaggggtaatgaacgaagtggcagaagaggaccgaaggaaagtgattgaactccacagctactgcaccagcgtctacgaagagggtgatgctcgctctgctcttattgctatgttgcagtccctaaaccatgcaaagaatggagtggacattgtctctgggacacgagtcatgtctcactttgcaaaacccaattggcgcagtgtctacaagcgcattgcgcttaatcatccagatgcccgagttggtcagttcccaccccttctactcacgttgcgtctgctttttgcttttttccaaattcattaaattgcaatgcaggggtattttactgtgggccatcagccctcacccatgagcttcgtcagctagcattggatttctctcacaacacatccaccaagtacgacttccataaagaaaatttctgacaagatcgagctaagccgtccaccacctagatcattccactttgttttttgcctataaataattaagtttctacatgtggttcttatttatggctttgtaaaagcaccaaggcaggtgggagctaaatggttaaaatagagttgcctaccaattataaaaggtgtggacgtctgttaatgtctctgcgtgtctttatgtccatgtaaatttaaggagaaataaccactccttggaaacaccttgggagaaactagaaagtaatacagaagagagaataatgtctagctggagcgcacttgatactttgtattaatccccgatgccttttagctgatcaattgcagaattcaggagattcatttataaatattataaaaaaaaaaaaaaaaa

SEQ ID NO: 42 - protein, 616 aa, Glycine max
HENKLSNIQKQAEEYAALIMEELDPDDTGYIMIDNLETLLLHGPEETTRGESKYLSQMLSQKLKPTFADSAVMRWCRDAKYFLLDNWQRSWVLALWIGVMFGLFAYKFVQYRRKAAYEVMGHCVCMAKGAAETLKLNMALILLPVCRNTITWLRNKTKLGVVVPLDDNINFHKVIAVAIAVAVAVHSIYHLTCDFPRLLHASDEKYKLMQPFFGDRPSDYWYFVKSWEGVTGIIIVVLMAIAFTLANPRFRRGRAKLPKPFNKFTGFNAFWYSHHLFVIVYALLVVHGIKLYLTKEWYKKTTWMYLAIPITIYALERLVRAFRSSIKSVKILKVTLYPGNVLSLKMSKPQGFSYKSGQYMFVNCAAVSPFEWHPFSITSAPDDDYLSVHIKILGDWTRSLKAKFTQACQQPLNGQSGLLRAECLKGDNSPSSFPKVLVDGPYGAPAQDYREYEVVLLVGLGIGATPMISILKDMVNNFKANDEEEGGQERVSDFKTRRAYFYWVTTHHGSFDWFKGVMNEVAEEDRRKVIELHSYCTSVYEEGDARSALIAMLQSLNHAKNGVDIVSGTRVMSHFAKPNWRSVYKRIALNHPDARVGQFPPLLLTLRLLFAFFQIH

SEQ ID NO: 43 - DNA, 1993 bp, Triticum aestivum
gcacgaggctgagccagcacctccggccgacggcggagcccaacccgctccggcgctggtaccgccgcgccagctacttcctcgaggacaactggcggcgctgctgggtgatcctgctgtggctctccatctgcgtgggcctcttcacgtggaagttcatgcagtaccgcgagcgcgccgtgttcaaggtgatgggctactgcgtgtgcgtggccaagggcggcgccgagacgctcaagttcaacatggcgctcatcctgctccccgtgtgccgcaacaccatcacgtggttccgcaaccgcaccgccgcgggccggttcgtgccgttcgacgacaacatcaacttccacaaggtgatcgccgcggggatctcggtcggcgcggggctgcacatcatctcccatttgacgtgcgacttcccgcgcctgctgcacgccaccgaggaggagtacgagcccatgaagcggttcttcggggatgaccagccgcccaactactggtggttcgtcaagggcacggaggggtggacggggctggtgatgctggtgctcatggcgatcgccttcacgctcgccatgccgtggttccgccgcggcaggctcagcctccccaagccgctcaaccggctcaccgggttcaacgccttctggtactcgcaccacctcttcgtcatcgtctacgcgctgctcatcgtccacggccacttcctctacctcaccaagaagtggcaaaagaagtcgacgtggatgtacctggcggtgccgatggtgatgtacgcgtgcgagcggctgacgcgggcgctgcggtcgagcgtgcggccggtgaagatactcaaggtggcggtgtaccccggcaacgtgctgtcgctgcacttctccaagccgcaggggttccggtacaagagcgggcagtacatcttcgtcaactgcgccgccgtctcgccgttccaatggcacccgttctccatcacgtcggcgccgcaggacgactacgtgagcgtgcacatcaggacgctgggggactggacccgggagctcaagaacgtcttctccaaggtctgccggccgccgacggagggcaagagcggcctgctccgggccgagtacgaccgcgacgtcggcgccatgtccaacccgagcttcccaaaggtgctgatcgacggcccctacggcgcgccggcgcaggactacaagcaatacgacatcgtgctgctcgtggggctgggcatcggggccacacccatgatctccatcatcaaggacatcatcaacaacatgaagcggctcgaaggagacgtcgagtccggcaaccccggggacgcgagcacgtcggcgtccttccggacccggcgcgcctacttctactgggtgacgcgggagcaaggctccttcgagtggttccggggcgtcatggacgagatagctgagtcggacaagaagagtgtcatcgagctccataactactgcaccagtgtctacgaggacggggacgcccggtccgcgctcatcgccatgctccagtccctcaaccatgccaagaacggcgtcgacatcgtctccggcacccgtgtcaagacccactttgcgcgaccaaactggcgcaacgtctacaagcgtatcgccctcaaccaccgtgaacagcgtgtcggagtattctactgcggtgcaccggtgctaacaaaggagctgcgtgaccttgcacaagatttctcgagaaagacaaacacaaaattcgagttccacaaggagaatttttaacttctgtagacgacccagccacaagaagttgctctttttgccgaaagtgtgttaattcagtacatagcaagttcttatacttgtgagtagagcaactagatcaaaggagatattgaatgtgttgatttattagtacatagcaagtcttgtacttatgaaaagtagaggggctagatcaaagtaggtattggttgattataacaagtttgatcagatttaaaaaaaaaaaaaaaaaa

SEQ ID NO: 44 - protein, 584 aa, Triticum aestivum
TRLSQHLRPTAEPNPLRRWYRRASYFLEDNWRRCWVILLWLSICVGLFTWKFMQYRERAVFKVMGYCVCVAKGGAETLKFNMALILLPVCRNTITWFRNRTAAGRFVPFDDNINFHKVIAAGISVGAGLHIISHLTCDFPRLLHATEEEYEPMKRFFGDDQPPNYWWFVKGTEGWTGLVMLVLMAIAFTLAMPWFRRGRLSLPKPLNRLTGFNAFWYSHHLFVIVYALLIVHGHFLYLTKKWQKKSTWMYLAVPMVMYACERLTRALRSSVRPVKILKVAVYPGNVLSLHFSKPQGFRYKSGQYIFVNCAAVSPFQWHPFSITSAPQDDYVSVHIRTLGDWTRELKNVFSKVCRPPTEGKSGLLRAEYDRDVGAMSNPSFPKVLIDGPYGAPAQDYKQYDIVLLVGLGIGATPMISIIKDIINNMKRLEGDVESGNPGDASTSASFRTRRAYFYWVTREQGSFEWFRGVMDEIAESDKKSVIELHNYCTSVYEDGDARSALIAMLQSLNHAKNGVDIVSGTRVKTHFARPNWRNVYKRIALNHREQRVGVFYCGAPVLTKELRDLAQDFSRKTNTKFEFHKENF

SEQ ID NO: 45 - DNA, 672 bp, Zea mays
caagctggtacgcctgcaggtaccggtccggaattcccgggtcgacccacgcgtccgctgccaataagctgtccaggcttaaggagcaagccgaagagtatgccgccctgattatggaggagcttgatcctgaaggacttggctacattgagttgtggcagttggagacccttctgttgcagaaggatacctacatgaactacagtcaggcgctaagttacacaagccaagcactaagtcagaacctagcaggcttaaggaaaaagagtccaattcgcaagataagtaccacattgagttactatttggaggataattggaaacgcctatgggtgcttgcattatggattggaataatggctggactgttcacttggaagttcatgcagtatcgcaacaggtatgtctttaatgttgatgggctactgtgtgaccactgcaaaaggcgctgcttgaaaccctgaagctgaacatggcaattatcctcctcccagtatgccggtaacaccattactttggttgagaaatacaagggctgcacgggcattgccatttgatgaatatattaacttcccaccagactattgcagcagcatcgtttgttgggtatatcctcccatgccaggaacctcctgtgttgtgaatttcccacggctataattcctccaatga

SEQ ID NO: 46 - protein, 199 aa, Zea mays
ASAANKLSRLKEQAEEYAALIMEELDPEGLGYIELWQLETLLLQKDTYMNYSQALSYTSQALSQNLAGLRKKSPIRKISTTLSYYLEDNWKRLWVLALWIGIMAGLFTWKFMQYRNRYVFNLMGYCVTTAKALLETLKLNMAIILLPVCRLLWLRNTRAARALPFDEYINFPPDYCSSILLGISSHARNLLCCEFPTAI

SEQ ID NO: 47 - DNA, 432 bp, Oryza sativa
  unsure (346)
  unsure (351)
  unsure (368)
  unsure (399)..(400)
  unsure (406)
  unsure (430)
accttgctggtctaaggaaaaggagtccaatccggaaaataagcaccaaattaagctactatctggaggacaactggaagcgcctgtgggtacttgcactgtggattgggataatggctgggttgttcatttggaaattcatacaataccgccaccgatatgtctttaacgtgatgggctactgtgtaacaactgcaaaaggggctgctgagactcttaagctgaatatggctattatcctcctgccagtatgcccgcaacaccatcacttggttgaggaatacaagggctgcacgggcattgccgttcgatgacaacatcaacttccacaaagactattgcaacnacaanttgtgggttggggttanccttcaaggggggggccccaaccttggtagnntatttncccccgggctccaaagggttccancg

SEQ ID NO: 48 - protein, 110 aa, Oryza sativa
LAGLRKRSPIRKISTKLSYYLEDNWKRLWVLALWIGIMAGLFIWKFIQYRHRYVFNVMGYCVTTAKGAAETLKLNMAIILLPVCRNTITWLRNTRAARALPFDDNINFHK

SEQ ID NO: 49 - DNA, 558 bp, Glycine max
  unsure (485)
  unsure (504)
  unsure (534)
  unsure (540)
  unsure (546)
  unsure (551)
  unsure (557)..(558)
agacactgaagactacaaacgcttatttttactgggtaacaagagaacaaggttctttcgattggtttaaaggggtcatgaatgaagttgctgaacttgatcaaaggggtgttattgagatgcacaactacttaactagcgtatatgaggaaggcgatgccagatccgcccttattacaatggtacaagccctcaaccatgcaaaaaatggagttgatattgtttctgggactagggtaagcctttgatttttgccctttactttaccctttatgcgttttagttttggaaaaatagaagtatcctaaagagtttgcttttcagtcttgagtaagttgattttaaaccttgatttgcttctctgaatttactatcaggtgagaactcattttgccagggcctaactggaagaaggtttctcaagaatatgctctaaagcactgcaatggacgaataaggggtttcctaattggtggggcaccggntttggccaaaagaatttancaagctcctgctccgaggtcaacggaaaanggcaancaaaantttgngtcccnn

SEQ ID NO: 50 - protein, 78 aa, Glycine max
LKTTNAYFYWVTREQGSFDWFKGVMNEVAELDQRGVIEMHNYLTSVYEEGDARSALITMVQALNHAKNGVDIVSGTRV

SEQ ID NO: 51 - DNA, 2224 bp, Zea mays
ccacgcgtccgctgccaataagctgtccaggcttaaggagcaagccgaagagtatgccgccctgattatggaggagcttgatcctgaaggacttggctacattgagttgtggcagttggagacccttctgttgcagaaggatacctacatgaactacagtcaggcgctaagttacacaagccaagcactaagtcagaacctagcaggcttaaggaaaaagagtccaattcgcaagataagtaccacattgagttactatttggaggataattggaaacgcctatgggtgcttgcattatggattggaataatggctggactgttcacttggaagttcatgcagtatcgcaacaggtatgtctttaatgtgatgggctactgtgtgaccactgcaaaaggcgctgctgaaaccctgaagctgaacatggcaattatcctcctcccagtatgccgtaacaccattacttggttgagaaatacaagggctgcacgggcattgccatttgatgataatattaacttccacaagactattgcagcagcaatcgttgttggtataatcctccatgcagggaaccatcttgtgtgtgattttccacggcttataaattcatcaaatgagaaatatgctcctctgggccagtacttcggtgaaacaaagccaacatactttacattggtaggagtggaaggcatcactggtgtaattatggttatctgtatgattattgctttcactttagctacacggtggttccgtcgtagcctggtgaagcttccaaaaccatttgacaaactgactggcttcaatgccttctggtactctcatcatttattcataattgtgtatttggctctcattgttcatggccagttcctttacctcattcatgtctggtaccgaaaaacgacatggatgtatctggcagtgcctgtttgcctgtatgtaggggagagggtgctgaggttcttcaggtctggcagttattctgtcaggctattgaaggtggccatatatcctggtaatgtgttgacactgcaaatgtccaagcctccggctttccgatacaagagtgggcaatatatgtttgttcaatgtccagcagtgtcaccctttgaatggcatccgttctcaattacttcagcgcctggggatgattatctaagcattcatgttcgacaacttggtgactggacacgagagctcaagagggtttttgcagcagcttgtgaacccccagtgggcggtaaaagtggccttcttagagcagacgagacaaccaagaaagccttaccaaaacttctgattgatggaccttatggttctcctgctcaggattacagcaagtatgatgttcttctacttgttggattagggattggtgcaactccttttatcagcatactgaaagatcttcttaacaatattataaggatggaggaagaggaggatgcttccactgatctttatcctccagttggtccgaacaagccgcacattgatctcagcacacttatgacggtcacatcaagaccaaagagggttctaaggaccacaaacgcttacttctattgggttacacgtgagcaaggttcttttgattggttcaagggagtcatgaatgaaattgctgaactggaccaaaggaatattattgagatgcacaactaccttacgagtgtttatgaggaaggagatgctcggtcagcgctcatcaccatgcttcaagctttgaaccatgcaaagaatggcgttgacattgtctctggaactaaagtccggacacactttgcaagaccaaattggaaaaaagtcctatctaaaattgcctcgaagcatccatttgctaagataggtgtattctactgtggagctccagttcttgcacaagaactaaacaaactgtgccatgaattcaatgggaaaagcacaacgaaatttgagttccacaaggaacatttctgaattccagtaatacttggtgcgcagaaatgatatattatgtgtgtgtacagcatttttgtcttggtatgttcatctggagttctggacgaacatgagagcaaagtggagaagctaccatcaaatgcttaacagctgacggcctgcactctttgttaactgctcctaaatgaaccagaccaccaagaagtgggatttttgtgtacataaaaaaaaaaaaaaaaaaaaaaaaaaaaaaaaaaaaaaaaaaaaaaaaaaaag

SEQ ID NO: 52 - protein, 654 aa, Zea mays
HASAANKLSRLKEQAEEYAALIMEELDPEGLGYIELWQLETLLLQKDTYMNYSQALSYTSQALSQNLAGLRKKSPIRKISTTLSYYLEDNWKRLWVLALWIGIMAGLFTWKFMQYRNRYVFNVMGYCVTTAKGAAETLKLNMAIILLPVCRNTITWLRNTRAARALPFDDNINFHKTIAAAIVVGIILHAGNHLVCDFPRLINSSNEKYAPLGQYFGETKPTYFTLVGVEGITGVIMVICMIIAFTLATRWFRRSLVKLPKPFDKLTGFNAFWYSHHLFIIVYLALIVHGQFLYLIHVWYRKTTWMYLAVPVCLYVGERVLRFFRSGSYSVRLLKVAIYPGNVLTLQMSKPPAFRYKSGQYMFVQCPAVSPFEWHPFSITSAPGDDYLSIHVRQLGDWTRELKRVFAAACEPPVGGKSGLLRADETTKKALPKLLIDGPYGSPAQDYSKYDVLLLVGLGIGATPFISILKDLLNNIIRMEEEEDASTDLYPPVGPNKPHIDLSTLMTVTSRPKRVLRTTNAYFYWVTREQGSFDWFKGVMNEIAELDQRNIIEMHNYLTSVYEEGDARSALITMLQALNHAKNGVDIVSGTKVRTHFARPNWKKVLSKIASKHPFAKIGVFYCGAPVLAQELNKLCHEFNGKSTTKFEFHKEHF

SEQ ID NO: 53 - DNA, 2480 bp, Oryza sativa
cactaagccagaaccttgctggtctaaggaaaaggagtccaatccggaaaataagcaccaaattaagctactatctggaggacaactggaagcgcctgtgggtacttgcactgtggattgggataatggctgggttgttcatttggaaattcatacaataccgccaccgatatgtctttaacgtgatgggctactgtgtaacaactgcaaaaggggctgctgagactcttaagctgaatatggctattatcctcctgccagtatgccgcaacaccatcacttggttgaggaatacaagggctgcacgggcattgccgttcgatgacaacatcaacttccacaagactattgcagcagcaattgtggttggtgttatccttcatggagggctccatcttgtatgtgattttccacggctcataggttcatcggaggagaagtatgctccactagggaagtattttggtgaaactaagccaacatatttgaccctggtcaaaggagtggagggcataactggggtaatcatgcttgtgtgcatgattatcgcttttactcttgcaaccaggtggttccgccgtagcctggtgaagcttccaaagccatttgacaaattaactggcttcaacgctttctggtattctcaccatctattcatcattgtgtacatatcacttgtaattcatggagagtggctataccttatccgcatatggtacaaaaggacgacatggatgtatcttgcagtgcccgtttgtttgtatgtaggggagaggacactgaggttcttcaggtctggcagttattctgtccgtctgttgaaggtggccatctatcctggtaatgttttaacactgcagatgtctaagcctcccacattccgttataagagtgggcagtatatgtttgttcaatgtccagctgtttcaccctttgaatggcatcccttctcaataacttcggcacctggggatgactatctcagcattcatgttcggcaacttggtgactggacaagagagctcaagagggtgttctcagcggcttgtgagccaccagtgggtgggaaaagtggtctccttagagcagatgagactaccaagaaagccttacccaaattgttgattgatggtccatatggctctcctgcgcaagactacagcaagtatgatgttttactgctggttggattaggaattggcgcaacgccttttatcagcatattgaaagaccttatcaacagcatcatcaaaatggaggaagaggaagaagcttcaggtgatctttacccgccaatcggacgcaataaagcacatgttgatcttgacacccttatgaggattacctcaaaaccaaagagggttttgaagacaacaaatgcttatttttattgggtgacacgtgaacaaggctcttttgattggtttaaaggagtcatgaatgagattgctgaactagatcaaaggaatattattgagatgcacaactacctcacaagtgtttacgaggaaggggatgctcggtcagcactcattactatgctgcaagctctaaaccatgccaagaatggtgttgatatagtatctggaactaaagtccgtacacattttgcaaggccaaattttaagaaggttctttctaagatagcctccaaacatccttatgctaaaataggagtattctactgtggggctccagttctggctcaggaattaagcgatctttgccatgattttaatggcagatgcacgtcaaaatttgagtttcacaaggagcatttctaaaatcaggtaaatgccaaatagggaaataatagaagattgtgctacttgtatgacaccttttcttttacacgatgccttgttcacaatggaattaatagcaagggcgagaagcatagaggtgctatcacaacgcaccctcctggctacggccagtttgcactgtacacttcttgcttgagatactcagaagatacaataagagtgccaagataagagttttggggatcatctggtacaaaatatataggacatcaatcatggggaaacataggttccaacttccaagtgcatcggcgaggtgattgctcgtaacatgttgatctcctggatttggatttggttttcgactataatctggtgtaggacatagaaggctaccatgagcacaaggctaattaattgtatattgcgtgcagctgagtggtcaggctacaagatttgctctaactttgtaattatatacagagatgggaaacaaacaaatatagtgtgatttttgtaagtagtgtaggttggtagtgattgggtccagattggtggctcagattacattcaagcttgtttttttgttgtgccgggagtgtacaacgtgcattgttttagggatggttttacacacatacagatgatgtaatcatgtaatccttgtagcaaaggccttgttcttacgttggaaaaaaaaaaaaaaaaaaaaaa

SEQ ID NO: 54 - protein, 593 aa, Oryza sativa
LSQNLAGLRKRSPIRKISTKLSYYLEDNWKRLWVLALWIGIMAGLFIWKFIQYRHRYVFNVMGYCVTTAKGAAETLKLNMAIILLPVCRNTITWLRNTRAARALPFDDNINFHKTIAAAIVVGVILHGGLHLVCDFPRLIGSSEEKYAPLGKYFGETKPTYLTLVKGVEGITGVIMLVCMIIAFTLATRWFRRSLVKLPKPFDKLTGFNAFWYSHHLFIIVYISLVIHGEWLYLIRIWYKRTTWMYLAVPVCLYVGERTLRFFRSGSYSVRLLKVAIYPGNVLTLQMSKPPTFRYKSGQYMFVQCPAVSPFEWHPFSITSAPGDDYLSIHVRQLGDWTRELKRVFSAACEPPVGGKSGLLRADETTKKALPKLLIDGPYGSPAQDYSKYDVLLLVGLGIGATPFISILKDLINSIIKMEEEEEASGDLYPPIGRNKAHVDLDTLMRITSKPKRVLKTTNAYFYWVTREQGSFDWFKGVMNEIAELDQRNIIEMHNYLTSVYEEGDARSALITMLQALNHAKNGVDIVSGTKVRTHFARPNFKKVLSKIASKHPYAKIGVFYCGAPVLAQELSDLCHDFNGRCTSKFEFHKEHF

SEQ ID NO: 55 - DNA, 1255 bp, Glycine max
gcacgagagacactgaagactacaaacgcttatttttactgggtaacaagagaacaaggttctttcgattggtttaaaggggtcatgaatgaagttgctgaacttgatcaaaggggtgttattgagatgcacaactacttaactagcgtatatgaggaaggcgatgccagatccgcccttattacaatggtacaagccctcaaccatgcaaaaaatggagttgatattgtttctgggactagggtaagcctttgatttttgccctttactttaccctttatgcgttttagttttggaaaaatagaagtatcctaaagagtttgcttttcagtcttgagtaagttgattttaaaccttgatttgcttctctgaatttactatcaggtgagaactcattttgccaggcctaactggaagaaggttttctcaagaatatgctctaagcactgcaatggacgaataggggttttctattgtggcgcaccggttttggccaaagaattaagcaagctctgcttcgagttcaacgaaaaaggtcaaacaaaatttgagttccacaaggagcatttctaagggaatttggagggagcttcatatctgtatctagaatcaacacaattaattttagcaactagtcgtagtcaattataagatatggttcaataagctaaaaccttaagtatagaatatgaggcattgtacatcccggccggccggcaagatggctgggcattgatcatacatgtggctgcagctcaaaggagatattgcccttacaaggctccagcaacttcagatacattctgccgaaccgcatagattattggccgatacatcccatagaaaatatacaacagttttgaaagtgataaaaaggaattagttagtgttctgcctcaggagcaatgcattggtttgtggataatttatttttttaaaaattatcgttttctaatatttattttaaaaaatatacttgaaaatgattttttttttttaagtaaatgtcaacaaacatataccaaattttcgttttcttcgagtggattccattatctttgtggaatattgtgggtgaaactattgtttcgaccctgctgcttctgcttggatttcttctccgggagaggagatctaatgtctaaacactgtacatgagatgagactcttaatcgtatgttgactatgttctttttcttaaaaaaaaaaaaaaaaaaaaaaaaaaaaaaaaaaaaacaaaaaaaaaaaaaaaaaa

SEQ ID NO: 56 - protein, 143 aa, Glycine max
ARETLKTTNAYFYWVTREQGSFDWFKGVMNEVAELDQRGVIEMHNYLTSVYEEGDARSALITMVQALNHAKNGVDIVSGTRVRTHFARPNWKKVFSRICSKHCNGRIGVFYCGAPVLAKELSKLCFEFNEKGQTKFEFHKEHF

SEQ ID NO: 57 - DNA, 496 bp, Zea mays
gtctccttcacgctggccacgcacccgttgcgcaagggggagcccaagggcgcgggcgcggccgccggcacgtcgcggctcccggcgccactgaaccggctcaccggcttcaacgccttctggtactcgcaccacctcctcggcatcgtgtacgcgctcctgctcgcgcacggctacttcctcttcctcgtccggaggtggtacgagaagacgacatggatgtacatttcggtcccgctgctgctctacgtcggtgaaaggatgcttagagccttgaggtcaaatgcttataccgtgaaaattcttaaggtgtgtcttctacctgggaatgtgttgaccataacgatgtcaaagccctatggatttcgttacagaagtggacagtacatatttcttcaatgcccaataatttctccattcgaatggatcctttctccatcacttcagcacctggagatgactacctaagtgtcacatccgaacaaacgtgactgga

SEQ ID NO: 58 - protein, 133 aa, Zea mays
  UNSURE (118)
RLPAPLNRLTGFNAFWYSHHLLGIVYALLLAHGYFLFLVRRWYEKTTWMYISVPLLLYVGERMLRALRSNAYTVKILKVCLLPGNVLTITMSKPYGFRYRSGQYIFLQCPIISPFEWXPFSITSAPGDDYLSV

SEQ ID NO: 59 - DNA, 571 bp, Glycine max
  unsure (293)
  unsure (303)
  unsure (308)
  unsure (322)
  unsure (332)
  unsure (350)
  unsure (353)
  unsure (356)
  unsure (369)
  unsure (378)
  unsure (385)
  unsure (389)
  unsure (403)
  unsure (526)
ccaaatatatgatctattccaagaggcagtgttatcacgatcacaagggtgtccaaaactgtacatagatggcccttatggttctgctgctcaagaccatgtaaagtatgacattctagtactaattggtcttggcataggagccacacctttcattagcatccttaaagatgtagttaaaggtgtccagacaacgcaaaatgatcatagtggtctcagacaatgcagcttaacaaaaggtccattaaaagcatatctttattgggttacaagagagccaaactcttttggancgggtttaanatgtnatgaagggaatccncaatttcaanccaaaaagcattcggttntgnagntgcacatttcccngacaagtgnacatccngaangggggatattcgtncagctttgctaaagggtaatccgggccttgcaatcttggccaagaatgggcctgacatagtttccaagggcttcccatacacacacatttttgctcggaccaaattgggtccaacatatttcnaggattggctcgcaagcatgggaggagctaagattggggggtttc

SEQ ID NO: 60 - protein, 137 aa, Glycine max
  UNSURE (98)
  UNSURE (101)
  UNSURE (103)
  UNSURE (111)
  UNSURE (117)..(118)..(119)
  UNSURE (123)
  UNSURE (126)
  UNSURE (130)
QIYDLFQEAVLSRSQGCPKLYIDGPYGSAAQDHVKYDILVLIGLGIGATPFISILKDVVKGVQTTQNDHSGLRQCSLTKGPLKAYLYWVTREPNSFGXGLXCXEGNPQFQXKKHSVXXXHISXTSXHPEXGIFVQLC

SEQ ID NO: 61 - DNA, 493 bp, Triticum aestivum
  unsure (466)
  unsure (491)
ccacctcctcggcttcgtctacctcctcctcctcgcccacggctacttcctcttcctcgtccgccgctggtacgagaaaacgacatggatgtacatttctgtccctctggtgctctatgtcggcgaaaggatgctgcgagccttgcggtcgaatgctcaccctgtcaaaatcctcaaggtgttgcttctacctggaagtgtactgacaatacaaatgtcaaagccctacggatttcgatataggagtggacaatatatctttcttcagtgtccgatgatctctccatttgaatggcatcctttctccatcacctcagctcctggagatgactacctcgctgttcacattcgcacaaacggagactggacgcaagagctcaagcgcatatttgtcgagaactacttcacgccgcacatgaacagaagaactcaatcagcgagttaggcgcggcagaacctagaccantcccgcacaaagttgctcgtagatngg

SEQ ID NO: 62 - protein, 129 aa, Triticum aestivum
HLLGFVYLLLLAHGYFLFLVRRWYEKTTWMYISVPLVLYVGERMLRALRSNAHPVKILKVLLLPGSVLTIQMSKPYGFRYRSGQYIFLQCPMISPFEWHPFSITSAPGDDYLAVHIRTNGDWTQELKRI

SEQ ID NO: 63 - DNA, 1703 bp, Zea mays
gcacgaggtctccttcacgctggccacgcacccgttgcgcaagggggagcccaagggcgcgggcgcggccgccggcacgtcgcggctcccggcgccactgaaccggctcaccggcttcaacgccttctggtactcgcaccacctcctcggcatcgtgtacgcgctcctgctcgcgcacggctacttcctcttcctcgtccggaggtggtacgagaagacgacatggatgtacatttcggtcccgctgctgctctacgtcggtgaaaggatgcttagagccttgaggtcaaatgcttataccgtgaaaattcttaaggtgtgtcttctacctgggaatgtgttgaccataacgatgtcaaagccctatggatttcgttacagaagtggacagtacatatttcttcaatgcccaataatttctccattcgaatggcatcctttctccatcacttcagcacctggagatgactacctaagtgtccacatccgaacaaacggtgactggacacaggagctcaagcgcatatttgtggagaactacttctcgccgcatctcaacagaagagcttcgtttagcgagctaggtgcggcagaaccaagaagcttgccaaaattactcgtagatggtccctatggtgcccctgcgcaggattttagaaactacgacgttctacttctcgtcggcctcggaatcggggcaacaccgttcataagcattctacgggatctgcttaataacattaagatagctgacgagctgatggacttggcaatggagaccagcaggtctgaagacagcgccaacagctttagcgtctcaacggcgagtagcaaccggaagagagcatacagaacaagccgtgcacatttctactgggtcacccgagaagccggatcctttgaatggttcaaaggggtgatgaatgaggttgcagaaatggacaagaagggtatcatagagctgcacaattacctcactagcgtttacgaggaacgcgacgcacggacaactctgctgtccatggtccaggctctgaaccacgccaagcacggcgtcgatatcgtatcggggaccagggtgaggacgcatttcgccagacccaactggaaaggagtcttcaacaagattgcctccaagcatccgaattcaacagttggtgtgttctactgcggcgcaccgacgcttgccaaggagctgaaggctctggcgcacgagatgagccacaggacgggcactcgcttccatttccacaaggagtacttctgagtttcgacggatagaatgggcagtcgatgctcgttctaacgtgatctggtcttttctgatctgatctcgatagcgttaggtaccggtaaagaagacagaattttgcacagatgcaagacaaaccaagctgacctctcctcctgatctggaactgtacatagcataaaagaagcaccacagggttagcttagtatacgtagcacaattcaaataaatatattgacaaagaaaagagagacagagaaagggcaggatagaggctgttgttgttaactcgaagatattgatgataggcatggcggtatacgtgtatacattatagttaggtgctaggtggtagttatatatatactgtatatgatatgggtcgatgaatatacgaggagccacatgctgctgccaaaaaaaaaaaaaaaaaaaaaaaaaaaaaaaaaaa

SEQ ID NO: 64 - protein, 421 aa, Zea mays
HEVSFTLATHPLRKGEPKGAGAAAGTSRLPAPLNRLTGFNAFWYSHHLLGIVYALLLAHGYFLFLVRRWYEKTTWMYISVPLLLYVGERMLRALRSNAYTVKILKVCLLPGNVLTITMSKPYGFRYRSGQYIFLQCPIISPFEWHPFSITSAPGDDYLSVHIRTNGDWTQELKRIFVENYFSPHLNRRASFSELGAAEPRSLPKLLVDGPYGAPAQDFRNYDVLLLVGLGIGATPFISILRDLLNNIKIADELMDLAMETSRSEDSANSFSVSTASSNRKRAYRTSRAHFYWVTREAGSFEWFKGVMNEVAEMDKKGIIELHNYLTSVYEERDARTTLLSMVQALNHAKHGVDIVSGTRVRTHFARPNWKGVFNKIASKHPNSTVGVFYCGAPTLAKELKALAHEMSHRTGTRFHFHKEYF

SEQ ID NO: 65 - DNA, 2312 bp, Triticum aestivum
gcacgagccacctcctcggcttcgtctacctcctcctcctcgcccacggctacttcctcttcctcgtccgccgctggtacgagaaaacgacatggatgtacatttctgtccctctggtgctctatgtcggcgaaaggatgctgcgagccttgcggtcgaatgctcaccctgtcaaaatcctcaaggtgttgcttctacctggaagtgtactgacaatacaaatgtcaaagccctacggatttcgatataggagtggacaatatatctttcttcagtgtccgatgatctctccatttgaatggcatcctttctccatcacctcagctcctggagatgactacctcgctgttcacattcgcacaaacggagactggacgcaagagctcaagcgcatatttgtcgagaactacttcacgccgcacatgaacagaagaacttcattcagcgagttaggcgcggcagaacctagacccactcccgcaccaaagttgctcgtagatggtccatatggtgcccctgcacaggatttcagaaactacgatgttctgcttctagttggccttggaattggagcaacacgttcataagcattctgaaggacctacttaacaatattaagctagctgatgagcttatggacttggcaatggagactactcaaactagtaggtctgaggacagtgccaacagcttcagtgtctcaactgctagcagcaacaggaagagatcatatagaacaagccgtgcacatttttactgggttactcgcgagcccatgtcatttgaatggttcaaaggagtgatgaatgaggttgccgaaatggacaagaagggtgtcatagagttgcacaattatcttacgagcgtgtacgaggagcgtgatgcacgaacaactctgttgtcgatggtccaagctctaaaccatgccaaacatggtgtcgacatcgtctcgggcaccagggtgaggacacactttgccaggccaaactggagggaagtcttcaccaaaatcgccgccaagcagccgaattcaacagttggagtgttctactgtggcgctccgacgctggccaaagaactgaagaacctgtcgcacgagatgagccacaagacgtcgacgcgcttccatttccacaaggagtacttctgatgggaagaagaattgattcttctgatctgcaaaatagcgttaggcaccggtaaagaagaaggaattttttgcacagaagcaaagcaaactgacctctctctcttcctgatctggaactgtacatagtataaagaagaacagcctgttaggggagggtagttagtgccatttatatattattaacaaaaaagaaagtaaagcaaggctgctatatccaaaaggagaaggaaagaggcagggtaaagagttgggttaaatttcagatatactaatatacatgtacacagtatagttaggtagttacgtatgaaatgctagctaaagcatatttacaagggccggaggctgccaaaaaaaaaaaaaaaaaaaaaaaaaacggcactgtcaactcccggtgggaacacctcggtcgactccatcaacgccatcttgccggtcatgggttacgcccacaacactgtcaacaacaatgttggctctgcctccatcatcggcggctacgtctaccgctccatgactaacccatgcctcaacggcaggtacatttacgcggacttgtatgcacagtccatgtggtcagggatcgagacgccggagaacagtggggtgtacaatgtgacgccgctgacattcggctgctccaagacgtcaccaatcctgtgtgatgtcgcggccaagagcccgctgccgtcattgggctacatcttctcctttggcgaagacaacgccaaggacttatacttgcttaccagcaagggagtgtacagggtggtcgaccctagcagttgcaactacgcatgcccaatcaagagctccacgcaggaaggggtgcccccgcctacctcgtcgccaagctcggcattcaatgcgcaaacttccaccgtaccaatgacgttgttggcaggagtgttgcttgtcttgctgagcttgggcttttgagaactattcattttaagttcagaatgttttgcaaagttgccatatggttatggtgttttgatgttgtatattttggatactctctctatgtgtgcaagcgtgtaaatttttagattgtatttatcaaattaaaagtcaaatatgcgcgaaatggattgaatctgggattttcccaattgcgcaaaaattgcgtcttcactattttaaaaaaaaaaaaaaaaaa

SEQ ID NO: 66 - protein, 382 aa, Triticum aestivum
TSHLLGFVYLLLLAHGYFLFLVRRWYEKTTWMYISVPLVLYVGERMLRALRSNAHPVKILKVLLLPGSVLTIQMSKPYGFRYRSGQYIFLQCPMISPFEWHPFSITSAPGDDYLAVHIRTNGDWTQELKRIFVENYFTPHMNRRTSFSELGAAEPRPTPAPKLLVDGPYGAPAQDFRNYDVLLLVGLGIGATTFISILKDLLNNIKLADELMDLAMETTQTSRSEDSANSFSVSTASSNRKRSYRTSRAHFYWVTREPMSFEWFKGVMNEVAEMDKKGVIELHNYLTSVYEERDARTTLLSMVQALNHAKHGVDIVSGTRVRTHFARPNWREVFTKIAAKQPNSTVGVFYCGAPTLAKELKNLSHEMSHKTSTRFHFHKEYF

SEQ ID NO: 67 - DNA, 550 bp, Zea mays
aagcgccgaaccgcgttccaggtgatgggctactgcgtctgcgtcgccaagggtgccgctgagatcctcaagctcaacatggctctcatcctgctgcccgtctgccggaatacgctgacgacgctcaggtccaccgcgctcagccatgtcatacccttcgatgacaacatcaacttccacaaggtcatcgcgctgtccatcgcgatcgccacagcgatccacacgctcgcacacgtgacctgcgacttcccaaggctgatcagctgcccgacggacaagttcatggccaccttggggtccaacttccactacaagcagccgacttacctgggcttgctggagagcacacccggggttaccggaatcctcatgatcatcataatgtccttctccttcacgctggcaacacattccttcaggcggagtgtggtgaagctgccatcgccgctacaccaccttgccggtttcaatgccttctggtacgctcacactgctggtccttgcgttatgttctgctggtggtgcactcctacttcatattctcacagga

SEQ ID NO: 68 - protein, 181 aa, Zea mays
KRRTAFQVMGYCVCVAKGAAEILKLNMALILLPVCRNTLTTLRSTALSHVIPFDDNINFHKVIALSIAIATAIHTLAHVTCDFPRLISCPTDKFMATLGSNFHYKQPTYLGLLESTPGVTGILMIIIMSFSFTLATHSFRRSVVKLPSPLHHLAGFQCLLVRSHCWSLRYVLLVVHSYFIF

SEQ ID NO: 69 - DNA, 525 bp, Glycine max
tttagatctttcatctttgtatatatactctttcttcttcttgcttctgttttttttttttgttagcttaaagtgtgttgcatctgaatttttatttattccgaaggtatgaaggattcgaaggaattcgctctggaactgtttgatgctctgagtcgtaaacgaaggttgagaactgacaaaatcagcagggaagaactgttcgaattctggtcgcaaattaccgatcaaagttttgattcgcggctccagatcttcttcgacatgtaattattcggtcattattgttgttatcgatgctcaataatttaattaatgataagtaatcttcctaagtttactttttgttctttaattaagtctgcgacacctaatgacaaattaaacacagcattgtcaaatgtcaccgtttatttgctttaatatttaaatgtccgttaaagcgttgatgttttctttgcttgcaagttgtgatgcaattatgatcattacgtgcttttgtatttaaaataaatcataatcaac

SEQ ID NO: 70 - protein, 54 aa, Glycine max
GMKDSKEFALELFDALSRKRRLRTDKISREELFEFWSQITDQSFDSRLQIFFDM

SEQ ID NO: 71 - DNA, 1840 bp, Zea mays
ccacgcgtccgaagcgccgaaccgcgttccaggtgatgggctactgcgtctgcgtcgccaagggtgccgctgagatcctcaagctcaacatggctctcatcctgctgcccgtctgccggaatacgctgacgacgctcaggtccaccgcgctcagccatgtcatacccttcgatgacaacatcaacttccacaaggtcatcgcgctgtccatcgcgatcgccacagcgatccacacgctcgcacacgtgacctgcgacttcccaaggctgatcagctgcccgacggacaagttcatggccaccttggggtccaacttccactacaagcagccgacttacctgggcttgctggagagcacacccggggttaccggaatcctcatgatcatcataatgtccttctccttcacgctggcaacacattccttcaggcggagtgtggtgaagctgccatcgccgctacaccaccttgccggtttcaatgccttctggtacgctcaccacctgctggtccttgcgtatgtcctgctggtggtgcactcctacttcatattcctcaccagggagtggtacaagaagacgacatggatgtacctgattgtccctgtcctcttctatgcctgtgaaagagtcatcaggaaatttcgtgagaacaactaccatgcgggaattgtgagggcagcaatttatccgggagatgtgctctctattcacatgaagaagccacagggtttcaagtacaagagtgggatgtatctgtttgttaaatgcccagaagtctcgcccttcgagtggcaccccttctctataacttcggcaccaggcgatgactacttgagtgtgcatatccgtacgctgggtgactggacatccgaactgcggatgctttttgggaaggcttgccaggcacaagtaacttccaagaaggctacccttacaagacttgaaactacagttgtggcagacgcccagacagaggacactaggtttcccaaggtctacatagacgggccatacggtgcaccagcacaaaattacaggaaatatgacattcttctgcttattggccttggaataggagcaactcctttcatcagcatactgaaggatatgttgaacaacctaaaatccaacgaagaggtgggaagcatccacggctctgagataggcagcttcaagaacaatggtccaggaagggcttacttctactgggtcaccagagagcaaggatctttcgaatggttcaaaggagtcatgaatgaggttgcagggagcgatcacagcaatgttatagagatgcacaattacctgaccagcgtgtatgaagaaggtgacgcaaggtcagctctgattgccatggtacagtcacttcagcgtgctaaaaacggcgtggatatcgtctccggcagcaagattcgaacacattttgcaagaccaaactggaggaaggtattctgtgatttggccagcgcacacaagaactctcgcataggagttttctattgtggatctccgacgctcacaaaacaactgaaggatctttcgaaagaattcagccagacaaccacaacccggttccatttccacaaggaaaacttctgagacgacgtgtacccgaagatccgatggactggaaacataattgtatagggaaaaaatacgatagcattggcatagcagatttagttttacaagttttgatgtatgcgggattgtacaaaatatgtgtagaaagctagatgtcaccatcatacatagattctgaaatgcttgcagatatatatattgcattgcataagtgaaaccacttgcttcctaaaaaaaaaaaaaaaaaaaaaaag

SEQ ID NO: 72 - protein, 532 aa, Zea mays
TRPKRRTAFQVMGYCVCVAKGAAEILKLNMALILLPVCRNTLTTLRSTALSHVIPFDDNINFHKVIALSIAIATAIHTLAHVTCDFPRLISCPTDKFMATLGSNFHYKQPTYLGLLESTPGVTGILMIIIMSFSFTLATHSFRRSVVKLPSPLHHLAGFNAFWYAHHLLVLAYVLLVVHSYFIFLTREWYKKTTWMYLIVPVLFYACERVIRKFRENNYHAGIVRAAIYPGDVLSIHMKKPQGFKYKSGMYLFVKCPEVSPFEWHPFSITSAPGDDYLSVHIRTLGDWTSELRMLFGKACQAQVTSKKATLTRLETTVVADAQTEDTRFPKVYIDGPYGAPAQNYRKYDILLLIGLGIGATPFISILKDMLNNLKSNEEVGSIHGSEIGSFKNNGPGRAYFYWVTREQGSFEWFKGVMNEVAGSDHSNVIEMHNYLTSVYEEGDARSALIAMVQSLQRAKNGVDIVSGSKIRTHFARPNWRKVFCDLASAHKNSRIGVFYCGSPTLTKQLKDLSKEFSQTTTTRFHFHKENF

SEQ ID NO: 73 - DNA, 1528 bp, Glycine max
tttagatctttcatctttgtatatatactctttcttcttcttgcttctgttttttttttttgttagcttaaagtgtgttgcatctgaatttttatttattccgaaggtatgaaggattcgaaggaattcgctctggaactgtttgatgctctgagtcgtaaacgaaggttgagaactgacaaaatcagcagggaagaactgttcgaattctggtcgcaaattaccgatcaaagttttgattcgcggctccagatcttcttcgacatcgcttctgcaaataggttgtccagattgcaggaacaggctgaagaatatgcagctctaatcatggaagagttggaccccgaaggacttggctacatcgaggtgggaaatctttttatattccataatatattctaattaaatttatcaacacatgcatacacatagtgaaaataatgtgttcatgttatttcttttcataaagaaaacatacagacgattaatgagtagcatgtgtattgcgtggtgaacaggaataatccattactttagcattattgttttatataatttccacaagtctactaaaataaaataaaaacataagtttttcttttccttgttatataaggttttcttggcacattacgggaacaattgctcccgcaattccaaacataaaagaatttacttgtttttattttattttttataaatctttaaaaatatgcaatatagaaataataaggtatttttttgtaattatcaatacaaaaagaaaattatatatcgaatttaacagcacttaatttttacaggcaaatttgtttaacaaatactgacgagtttcttaaaaaaatattaaataaaaaattaaatttattaattagttaaaaatggtgtgtttattttttaatttttaacaagtacatttaaacatgaattagttgatgatgcttgtttgttagttagcttagtgtacattaatttcctagcttagtcagtgttgggctagggccgcacttatctaattagtcaacaatggaccaaattgtgaattgaacaagtcacccgaaatttcctagtgtagttcagccgttttcaagggtaaaaagaaaaagaaagacaaattaggcatattataacttataagtaaaaatttgcttgctgaatttaaaaaattgatttgatatacttacaacatcatttcattaattacgtaaaaaaatattattagtaatagactaatagggtgcactcattaacatgcaatcttatttcttcattcttttgccgtgtcaacaaatttaaattcagaccactttatccctagctaggtacgtaatataataattaagctgttatttctaataactcagttcatgcagtgcagcggccgcggtgctggttaatataaattattacagtgacctcattatttctttgatcaataagacatttttttccagaagatattaattataattttgagtagtagtggtttttgtctttgggcatgtcttcagagtggatggagagagat

SEQ ID NO: 74 - protein, 104 aa, Glycine max
IFIYSEGMKDSKEFALELFDALSRKRRLRTDKISREELFEFWSQITDQSFDSRLQIFFDIASANRLSRLQEQAEEYAALIMEELDPEGLGYIEVGNLFIFHNIF

SEQ ID NO: 75 - DNA, 486 bp, Zea mays
  unsure (456)
  unsure (465)
accaaatcccgatgttctgtgcaacacgagaataaagttcgggtctttcttagaagccattgaaaatttaggatttgactacatagcttcaggacactatgcacacgtagtacatccttccgctgaaaataccgaagcgccatccgtactacaattatcaaaggacaagatcaaggatcaaacctatttcctctcacatctatctcaatctcagcttagaaggcttcttttcccactaggatgtattacaaaggatgaggttcgcagactggctactcaaatgggccttcctaaccaaggtaggaaagactcacaagggatatgctttcttggaaaggtatacactgtacctttttttcttagggatcaatgtttttaacaaatccattggggagctatacaagtatcctctttctagggtcaagtttaagtgaagtttgttgaaaagacatatanggagaaaanggaagggaatcatactgggaa

SEQ ID NO: 76 - protein, 36 aa, Zea mays
PNPDVLCNTRIKFGSFLEAIENLGFDYIASGHYAHV

SEQ ID NO: 77 - DNA, 321 bp, Glycine max
  unsure (94)
  unsure (99)
  unsure (158)
  unsure (170)
  unsure (226)
  unsure (241)
  unsure (270)..(271)
  unsure (273)
  unsure (280)..(281)
  unsure (294)..(295)..(296)
  unsure (313)
  unsure (315)
cgcacacctcgcctcctctctccggcgacctggaccgctacctccgctgctccatgccgcagaactcgccgctccgagtcgcggttcttgtcancggangcgtcgatagcagcgtcgctctccggctgctccacgccgctggccactcctgcaccgcnttctacctcaanatatggttccaagaagatttcgagaacttttggtctgagtgcccctgggaagacgntttgaagtatgctcnagatgtttgtaatcaggttgatgtaccgnnanaagttgnncatttaaccgatnnntactggaacaatgtggntncttacc

SEQ ID NO: 78 - protein, 83 aa, Glycine max
  UNSURE (8)
  UNSURE (10)
  UNSURE (33)
  UNSURE (52)
  UNSURE (57)
  UNSURE (67)..(68)
  UNSURE (70)
  UNSURE (75)
  UNSURE (81)..(82)
LRVAVLVXGXVDSSVALRLLHAAGHSCTAFYLXIWFQEDFENFWSECPWEDXLKYAXDVCNQVDVPXXVXHLTDXYWNNVXXY

SEQ ID NO: 79 - DNA, 1412 bp, Zea mays
gcacgagaccaaatcccgatgttctgtgcaacacgagaataaagttcgggtctttcttagaagccattgaaaatttaggatttgactacatagcttcaggacactatgcacacgtagtacatccttccgctgaaaataccgaagcgccatccgtactacaattatcaaaggacaagatcaaggatcaaacctatttcctctcacatctatctcaatctcagcttagaaggcttcttttcccactaggatgtattacaaaggatgaggttcgcagactggctactcaaatgggccttcctaaccaaggtaggaaagactcacaagggatatgctttcttggaaagtttagtgagtttgttgaaagacatataggagaaaaggaaggaatcatactggaagctgagtccggagattacctagggatacaccgtgggttttggttttatacaattggtcagcggcaaggattacggcttcctggaggaccttggtacgttgtagagaaagatgttcaaaacaatgtggtttttgtatcaaggaattattattcattggataagcggaggcgcacatttcgtgttggatcactaaactggtttgataattgtggacctggaaacaatgagcaactcaaatgcaaggtgcgacatagccctgaatttcatgattgcactttggtaaaagagcgtagtgaagaaaatggggatgctttggtggtgcatctggctgaagatgaccagggcttggcagctggtcagtttgcagcgttctatagtgaaaatgtatgcctggggtcaggcataattttggattcatgggacaagatgagcttccctgtctgctcaagggcactcgaaatcgcaaagttggcggacaagtcgagcttagggaagccaataagaatcgtgaacctggaacatatcgtgaaacctgaacaacaggaggaaatcaaagttgcctgacagattggtgccgtaaccttctcctagctccgccagctgcccccttcaaatcaattcatcaatcaaccgtatcgcaggccctggaaatcgaaggatgaggggtcttctagaatctaccgatatatacccccagttggaaactccaggtccttcttggaaggatgatatagtggaatatggcatgacttggtgcgctcgtgttgcagaggaacgatgaagcttcgtgcgtggatagggttgatctgaattaactgttgacagtgattttacgatctcgcgggaaagcaacacaatagtttgggcacgtcctctcctgagtggaatgatctggattcagtagtagtcagtggcacttcattaaaactaatacagggaaatcgtgacgttgtatgatagtggtaagatattgtattgtgaattttgacgataatatcggtgcgtgattgtgcc

SEQ ID NO: 80 - protein, 319 aa, Zea mays
TRPNPDVLCNTRIKFGSFLEAIENLGFDYIASGHYAHVVHPSAENTEAPSVLQLSKDKIKDQTYFLSHLSQSQLRRLLFPLGCITKDEVRRLATQMGLPNQGRKDSQGICFLGKFSEFVERHIGEKEGIILEAESGDYLGIHRGFWFYTIGQRQGLRLPGGPWYVVEKDVQNNVVFVSRNYYSLDKRRRTFRVGSLNWFDNCGPGNNEQLKCKVRHSPEFHDCTLVKERSEENGDALVVHLAEDDQGLAAGQFAAFYSENVCLGSGIILDSWDKMSFPVCSRALEIAKLADKSSLGKPIRIVNLEHIVKPEQQEEIKVA

SEQ ID NO: 81 - DNA, 1667 bp, Glycine max
gcacgagcgcacacctcgcctcctctctccggcgacctggaccgctacctccgctgctccatgccgcagaactcgccgctccgagtcgcggttcttgtcagcggaggcgtcgatagcagcgtcgctctccggctgctccacgccgctggccactcctgcaccgctttctacctcaagatatggttccaagaagatttcgagaacttttggtctgagtgcccctgggaagacgatttgaagtatgctaaagatgtttgtaatcaggttgatgtaccgttagaagttgttcatttaaccgatgaatactggaacaatgtggtttcttacctcattgaagagtatagtagtggccgaacccctaaccctgatgttctgtgcaatacaagaattaagtttggtgcattcttggatgcgattggtggcatgggttttgactatgttgcctctgggcattatgcaaatgttatccacccatgttctgatcggagggatgaaccttctgttctggaactatcacaagacatggtaaaggatcaaacatacttcctttcacacctctcacaatcccagctgaagcaacttctttttccgcttggttgtattcccaaggatgaagtccgcaagcttgccacaaaatttgatctaccaaataaggatagaaaggattcccaaggaatatgctttttgggcaagataagattcagtgaatttgttgcaagacatattggggagagagaaggtgtcatactagaagctgagacaggagatttccttggcaaacatcgaggattttggttttatactattggtcagcgccagggtctacggctacctggaggcccatggtatgttgttgagaaggatgttaaaaacaatgtagtttttgtgtcaagaaattacttttcctttgacaaaagaaggcgtgtattccgtgttggctcttttaaatggcttagcgggttgcccccaggccagacaactcagctccaatgcaaggtgagacatggtcctggattttatgattgtagcttacaaatggaagtcgaagctgatggccaatgtcactctgctgttgtccgcatatctgaagatgatcaaggcctagcagctgggcagtttgcggcattctatgagggaagaacatgcataggttctggtgtgattttggagttctgggatgatcagagttttccagtttgtacaaaagctttagaaattgctagaatggaagataaatcaaagattgggaatccggtaaaaataaaagttaaaccagataacccgcaagaagtgtgtgattctgcagagttagcaagtaaaacatcacaagggaataagaaattaaagagttgctggcactgaacaaaacagactgacgtgtaagaggaatgcagcttccattttccctttcaattggccacaacagagcattggcaaatgggggtatacatgcagattttctgatccttcgttctgtaacttttttatatacatgtttagggaaacaagaagtattttatagagagaaggatagcgatttaaggtaggtgagaaattagtagcttctattattcggtgtttccaattaaaatgtgtatatactttgtcatggtttgcttgatactttattcagtttattgacaaatgtaaaaaaaaaaaaaaaaaaa

SEQ ID NO: 82 - protein, 361 aa, Glycine max
RVAVLVSGGVDSSVALRLLHAAGHSCTAFYLKIWFQEDFENFWSECPWEDDLKYAKDVCNQVDVPLEVVHLTDEYWNNVVSYLIEEYSSGRTPNPDVLCNTRIKFGAFLDAIGGMGFDYVASGHYANVIHPCSDRRDEPSVLELSQDMVKDQTYFLSHLSQSQLKQLLFPLGCIPKDEVRKLATKFDLPNKDRKDSQGICFLGKIRFSEFVARHIGEREGVILEAETGDFLGKHRGFWFYTIGQRQGLRLPGGPWYVVEKDVKNNVVFVSRNYFSFDKRRRVFRVGSFKWLSGLPPGQTTQLQCKVRHGPGFYDCSLQMEVEADGQCHSAVVRISEDDQGLAAGQFAAFYEGRTCIGSGVI

SEQ ID NO: 83 - DNA, 555 bp, Helianthus tuberosus
  unsure (41)
  unsure (394)
  unsure (448)
  unsure (462)
  unsure (476)
  unsure (510)
  unsure (519)..(520)
  unsure (543)
tcatcaccaaaatggcgaaaggagtgaagagaaacacgaancaggaagccattgattcatccacacattcttccgatacaaaaccatcgcctccgttaaagaaatgcaaaacatctccgtcggttgctgtttcggccgatgaagaggctcggttcgttggaaaaccggttccggctgagcaagcgagggggaaatggcctcatcgatatgaatcgaagaataaagtgaaggttatttcgtcatctgatggtgaggagaaggagatttttcaagctaagtgccattataccaaagctactgttgatggtatttcttttgatctttatgatgatgcttatgtcaaggctgaagaagggaaccagattacattgctcggatcgtggagatgtttganaccgttgataaagattatatttctccgctcagtggttttcagagctgaagatangttattaaagccangccactttcgacanagacatgtctatccgaatgaatatgacaatcacngatagttgnncaaataagttgtcactccccaangttgacttgtta

SEQ ID NO: 84 - protein, 765 aa, Helianthus tuberosus
FVGKPVPAEQARGKWPHRYESKNKVKVISSSDGEEKEIFQAKCHYTKATVDGISFDLYDDAYVKAEEGKPDYIARIVEMFETVDKELYFSAQWFFRAEDTVIKSQAHLIDKRRVFYSEMKDDNPLDSIVSKFKIVQLPPNVDLVEKEKALSSYDYYYDMQYSKPVTFTTLHKENLTTESGESSVVSDDACSNGVVESNNKNAKPTKINDSEKSEMTLLDLYSGCGAMSTGLCYGTNMAGVKLVTKWAVDINEHACESLKLNHAETQVRNEAADDFLLLLKEWEKLCKQFGLLGSKRDDTNVKSEESDSEEIDESRDPYKGEFEVQRLTAVCYGDPNKANKQKLHFKVRWKGFGPSYDTWEPVDGLSNCEESIKDFVIKGYKSRMLPLPGDVDFICGGPPCQGISGHNRFRNYTDPLKDPKNHQLVVYMDIIDFLKPKFVLMENVCDLVKFADGILGYHAVGRLVSMNYQTRMGIMAAGSYGVPQCRLRVFLWGANTMMNLPQFPLPTHEVVGRGVVPVEFKDCIVGSDNDNSYKLEKSIRLGDAISDLPEVTNNKGKDEMEYAGAPQTSFQKYIRLRKQALGKDSSKRMMLYDHRPLELNEDDYARVCHIPKIKGANFRDLPGVRVGKGNKVELDPDVERVLLPSGKPLVPNYAITFVRGTSKKPFGRLGMDDIVTTVVGRAEPHNQALLHPNQDRVLTIRENARLQGFPDHYKLCGPVKARYMQIGNAVSFSVSTGLGYSLAKAIQGVCTSKPIKLPHKFPDCL

SEQ ID NO: 85 - DNA, 605 bp, Zea mays
  unsure (307)
  unsure (325)..(326)..(327)
  unsure (329)
  unsure (357)
  unsure (364)
  unsure (382)
  unsure (390)
  unsure (393)
  unsure (489)
  unsure (534)
  unsure (537)
  unsure (569)
  unsure (575)
  unsure (596)
gggagtatggtggttcccccaagacagagttccagcgctacattcgacttggtcgtaaagacatgttggattggtcgtttggtgaggaggctggtccagatgaaggcaagctcttggatcaccagcccttacggcttaacaatgatgattatgagcgggttaagcaaattcctgtcaagaagggagccaacttccgtgacctaaagggtgtcaaggttggagcaaataatgttgttgagtgggatccagaagtcgaacgtgtgtacctttcgtctgggaaaccactggttcctgactatgcgatgtnattcatcaagggcaaatnnntnaagccattcgggcgcctgtggtgggacnagacggntcctacagtttgtgaccngagcaganctnataaccaggttatattgcatccgacttaagcaagagtcttgactatccgggagaacgccaagggtacaggggctttcccgattactacccgattgntttggaccgatcaaggagaagtatattcaagtccggaacgccaanggnagtccctggttgcacgggcactgggctactgnctgggncaagcctacctggggaatctnacgggatca

SEQ ID NO: 86 - protein, 184 aa, Zea mays
  UNSURE (98)
  UNSURE (104)..(105)
  UNSURE (115)
  UNSURE (117)
  UNSURE (123)
  UNSURE (126)..(127)
  UNSURE (136)
  UNSURE (159)
  UNSURE (174)..(175)
SPKTEFQRYIRLGRKDMLDWSFGEEAGPDEGKLLDHQPLRLNNDDYERVKQIPVKKGANFRDLKGVKVGANNVVEWDPEVERVYLSSGKPLVPDYAMXFIKGKXXKPFGRLWWDXTXPTVCDXSRXXNQVILHPTXARVLTIRENAKGTGAFPITTRLXWTDQGEVYSSPERQXXSLVARALGY

SEQ ID NO: 87 - DNA, 433 bp, Oryza sativa
  unsure (433)
cttacaatttttattaggctgtcattctaaggagaaattacccccttttccactgcctacgcatgaggcgattgtgaagaatggctgcccgttggcttttgagcgtaatttggttggttggcccaatgacacaccaatgcaactagcaagaccaattgtccttgaagacattctttctgatctcccagaagtggcaaatggggaaagccgtgatgaaatgctgtatgtaaagggtcctcaaactgaattccaaagatacattcggtcatttaatgttgaagtgcaaggtcccagagctcaatgttacaaaaagattccaaatcctcaaaattgttatgatcatcgaccattggtactgggataatgattactaccaaaagggatattgcaaattcccaaagaaaaaagggaagctaattttaagagacctttn

SEQ ID NO: 88 - protein, 141 aa, Oryza sativa
  UNSURE (121)..(122)
EVANGESRDEMLYVKGPQTEFQRYIRSFNVEVQGPRAQCYKKIPNPQNCYDHRPLVLGFLLGCHSKEKLPPFPLPTHEAIVKNGCPLAFERNLVGWPNDTPMQLARPIVLEDILSDLPEVXXLLPKGILQIPKEKREANFK

SEQ ID NO: 89 - DNA, 1042 bp, Triticum aestivum
gcacgagctggggtgctctttcttccatggtgctccctaagtatcctctgcctacatatgacgttgtagtacgtggaggagcacctaatgccttttcgcaatgtatggttgcatatgatgagacacaaaggccatccctgaagaaagctttgcttcttggtgatgcattttcagatttaccaaaggtcgaaaaccatcaacctaacgatgtaatggagtatggtggttcccccaagacagagttccagcgctacattcgacttggtcgtaaagacatgttggattggtcgtttggtgaggaggctggtccagatgaaggcaagctcttggatcaccagcccttacggcttaacaatgatgattatgagcgggttaagcaaattcctgtcaagaagggagccaacttccgtgacctaaagggtgtcaaggttggagcaaataatgttgttgagtgggatccagaagtcgaacgtgtgtacctttcgtctgggaaaccactggttcctgactatgcgatgtcattcatcaagggcaaatcactcaagccattcgggcgcctgtggtgggacgagacggttcctacagttgtgaccagagcagagcctcataaccaggttatattgcatccgactcaagcaagagtcttgactatccgggagaacgcaaggttacagggcttccccgattactaccgattgtttggaccgatcaaggagaagtatattcaagtcgggaacgcagtggcagtccctgttgcacgggcactgggctactgtctgggtcaagcctacctgggtgaatctgacgggagtcagcctctgtaccagctgcctgcaagttttacctctgtggggcgaaccgcggttcaggcgaatgccgtttctgttggcactcctgcgggggaggtagtcgagcagtaaaaggatagcggagcaaccctggttggtattttgattcgagcccatccagtagcatgtttaccaataaataatcattggtcgtgctgattcttatggttggagatgaatgtatgtagggtgta

SEQ ID NO: 90 - protein, 305 aa, Triticum aestivum
TSWGALSSMVLPKYPLPTYDVVVRGGAPNAFSQCMVAYDETQRPSLKKALLLGDAFSDLPKVENHQPNDVMEYGGSPKTEFQRYIRLGRKDMLDWSFGEEAGPDEGKLLDHQPLRLNNDDYERVKQIPVKKGANFRDLKGVKVGANNVVEWDPEVERVYLSSGKPLVPDYAMSFIKGKSLKPFGRLWWDETVPTVVTRAEPHNQVILHPTQARVLTIRENARLQGFPDYYRLFGPIKEKYIQVGNAVAVPVARALGYCLGQAYLGESDGSQPLYQLPASFTSVGRTAVQANAVSVGTPAGEVVEQ

SEQ ID NO: 91 - DNA, 681 bp, Triticum aestivum
gcacgaggtttcgtggaacagtcaaagacaggtatcgccagattgggaatgctgtggccgtacccgttggccgtgcacttgggtatgccttggccatggccaatctgaacaagactggaaatgatcccctcatggcgctaccacctaagtttgcattctctcataatatagaaggtactccttagtcagactggcaaaacctaacggttgacttgtccacccgggtttaagtctcgtcatctccgttcattctctgctagaaaagctgggtcatcgctgcacctcatggtcgtgaataccattaggactcgtgaatatcgttagggcatgcccattttaacacaagtcttgattcattttattgccatgttgtaccttgtaacatcttagtttcttagaagtttccgtaggcaggtgtaagttgctcagtaaggtttctcctgagagaaccttgtgttgtattgtaaaagaatgtatgtatcataactttgtcacacacatttgttagaagctcacatgggcaggtgtaggtggctgcagtgagttcctctgcctttgttgtactataaaagactctatgtatgtatgtatgtatgtatagacaagctgggtggtcaggggaataaaatgtacaccttctgaaaaaaaaaaaaaaaaaactcgagactagttctctctctc

SEQ ID NO: 92 - protein, 60 aa, Triticum aestivum
TRFRGTVKDRYRQIGNAVAVPVGRALGYALAMANLNKTGNDPLMALPPKFAFSHNIEGTP

SEQ ID NO: 93 - DNA, 2687 bp, Helianthus tuberosus
gcacgagtcatcaccaaaatggcgaaaggagtgaagagaaacacgaaacaggaagccattgattcatccacacattcttccgatacaaaaccatcgcctccgttaaagaaatgcaaaacatctccgtcggttgctgtttcggccgatgaagaggctcggttcgttggaaaaccggttccggctgagcaagcgagggggaaatggcctcatcgatatgaatcgaagaataaagtgaaggttatttcgtcatctgatggtgaggagaaggagatttttcaagctaagtgccattataccaaagctactgttgatggtatttcttttgatctttatgatgatgcttatgtcaaggctgaagaagggaagccagattacattgctcggatcgtggagatgtttgaaaccgttgataaagagttatatttctccgctcagtggtttttcagagctgaagatacggttattaaaagccaagcccaccttatcgacaaaagacgagtgttctattccgaaatgaaagatgacaatccactggatagtattgtatcgaaattcaagattgttcaacttcctccaaatgttgacttggttgagaaagagaaggcactctcatcgtatgattattactatgatatgcagtattcaaagccggttacatttacaactctacacaaagaaaacttaaccacagagagtggcgagtcatccgtagtttcagatgatgcgtgctcaaacggtgtagtagagagtaacaataaaaatgcaaaaccaacaaaaatcaacgacagtgaaaagtctgaaatgaccctattggacttgtattcgggttgtggtgccatgtcaaccgggctttgttatggcacaaatatggctggtgtaaaacttgtgacaaaatgggcagttgacatcaatgaacatgcatgtgaaagtctgaagctgaaccatgctgaaactcaggtgagaaatgaagcagctgatgactttttattgttgctgaaagaatgggagaaactttgtaaacaatttggtttgttgggttcaaaacgcgatgatacaaatgtaaaatctgaagaatcagattctgaggagattgatgagagtcgtgatccgtataagggtgaatttgaagtacaacgattgacggctgtttgttatggtgatccaaacaaagccaacaaacaaaagctgcattttaaggtgcgatggaagggctttggccctagttatgacacatgggagccagttgatggcttgagtaactgtgaggagtctataaaggattttgttatcaaaggatacaaatcaagaatgctaccacttcctggtgatgttgacttcatatgtggaggtcctccatgtcaaggaatcagtggtcataatcggtttagaaactatactgatcctttaaaggatccaaagaatcaccagcttgtagtctacatggatattattgattttttgaaaccaaaatttgttttgatggagaatgtttgtgatcttgtcaaatttgcggatggtattttgggatatcatgctgttgggcgtttagtttcgatgaattatcaaacacgtatggggataatggcagctggatcttatggagttcctcaatgcagactaagggtctttctttggggtgctaatacgatgatgaatttgcctcagtttccattaccaacacacgaggttgttggaagaggagttgttcctgttgagttcaaggactgtattgtcgggtctgataatgataactcgtacaagttagagaagagcatacgtcttggtgatgcaatttcagacttgcctgaggttacaaacaacaaaggtaaggatgaaatggagtatgcaggtgccccacaaacaagttttcagaaatacattagattgagaaagcaagctttggggaaagattcttcaaaaagaatgatgctttacgatcataggccattggaactaaacgaagatgattatgctcgggtttgccatattccaaagataaagggagcaaactttagggatttaccaggagtaagagttggtaaagggaacaaagtagaattggaccctgacgttgaaagggtcttgttaccttcaggaaaacctttggtcccaaattatgctataacatttgttcgtggaacttcaaaaaaaccctttggtcgcttgggtatggatgatattgtcacaactgtagttggtagagctgagccacataatcaggcgctgcttcatcctaaccaggatagagtgttgaccattcgtgaaaatgcacgtttacaaggatttcctgatcattataaactttgtggacctgttaaagcaaggtacatgcaaattggaaatgcagtttcattctcggtatcaactggattggggtacagcttagccaaagcaattcaaggagtttgcaccagtaaaccaatcaaactcccacataagtttccagattgtcttggacaactatcttcactataaacaagacattcaagggacaatgatgacaaaattggtttagtctttaatgaaagattgaggttaatttatgcatttaacttgcttatttcggcatcaattaattgtatgattttcatcatattagagtgtaattcaacattgattcttagtaagttttgttgatatgagcaactatgcttatattttttctagaaaaaaaaaaaaaaaaaaaa

SEQ ID NO: 94 - protein, 818 aa, Helianthus tuberosus
MAKGVKRNTKQEAIDSSTHSSDTKPSPPLKKCKTSPSVAVSADEEARFVGKPVPAEQARGKWPHRYESKNKVKVISSSDGEEKEIFQAKCHYTKATVDGISFDLYDDAYVKAEEGKPDYIARIVEMFETVDKELYFSAQWFFRAEDTVIKSQAHLIDKRRVFYSEMKDDNPLDSIVSKFKIVQLPPNVDLVEKEKALSSYDYYYDMQYSKPVTFTTLHKENLTTESGESSVVSDDACSNGVVESNNKNAKPTKINDSEKSEMTLLDLYSGCGAMSTGLCYGTNMAGVKLVTKWAVDINEHACESLKLNHAETQVRNEAADDFLLLLKEWEKLCKQFGLLGSKRDDTNVKSEESDSEEIDESRDPYKGEFEVQRLTAVCYGDPNKANKQKLHFKVRWKGFGPSYDTWEPVDGLSNCEESIKDFVIKGYKSRMLPLPGDVDFICGGPPCQGISGHNRFRNYTDPLKDPKNHQLVVYMDIIDFLKPKFVLMENVCDLVKFADGILGYHAVGRLVSMNYQTRMGIMAAGSYGVPQCRLRVFLWGANTMMNLPQFPLPTHEVVGRGVVPVEFKDCIVGSDNDNSYKLEKSIRLGDAISDLPEVTNNKGKDEMEYAGAPQTSFQKYIRLRKQALGKDSSKRMMLYDHRPLELNEDDYARVCHIPKIKGANFRDLPGVRVGKGNKVELDPDVERVLLPSGKPLVPNYAITFVRGTSKKPFGRLGMDDIVTTVVGRAEPHNQALLHPNQDRVLTIRENARLQGFPDHYKLCGPVKARYMQIGNAVSFSVSTGLGYSLAKAIQGVCTSKPIKLPHKFPDCLGQLSSL

SEQ ID NO: 95 - DNA, 1071 bp, Zea mays
ccacgcgtccgggagtatggtggttcccccaagacagagttccagcgctacattcgacttggtcgtaaagacatgttggattggtcgtttggtgaggaggctggtccagatgaaggcaagctcttggatcaccagcccttacggcttaacaatgatgattatgagcgggttaagcaaattcctgtcaagaagggagccaacttccgtgacctaaagggtgtcaaggttggagcaaataatgttgttgagtgggatccagaagtcgaacgtgtgtacctttcgtctgggaaaccactggttcctgactatgcgatgtcattcatcaagggcaaatcactcaagccattcgggcgcctgtggtgggacgagacggttcctacagttgtgaccagagcagagcctcataaccaggttatattgcatccgactcaagcaagagtcttgactatccgggagaacgcaaggttacagggcttccccgattactaccgattgtttggaccgatcaaggagaagtatattcaagtcgggaacgcagtggcagtccctgttgcacgggcactgggctactgtctgggtcaagcctacctgggtgaatctgacgggagtcagcctctgtaccagctgcctgcaagttttacctctgtggggcgaaccgcggttcaggcgaatgccgtttctgttggcactcctgcgggggaggtagtcgagcagtaaaaggatagcggagcaaccctggttggtattttgattcgagcccatccagtagcatgtttaccaataaataatcattggtcgtgctgattcttatggttggagatgaatgtatgtagggtgtactcgagctcgagtgcttgttgtactgtaggttgaggtttctcatccattggcctgcctatttgtggatgacgtttcatttcagattagcaatgtgcttatttaaggtttcgtcatgtacctgtattctacaatccactattgtttccaaagacagcatttgatccttgttcaacgcgagcaaaaaaaaaaaaaaaaaaaaaaaaaaaaaaaaaaaaaaaaaaaaaaaaaaag

SEQ ID NO: 96 - protein, 238 aa, Zea mays
PRVREYGGSPKTEFQRYIRLGRKDMLDWSFGEEAGPDEGKLLDHQPLRLNNDDYERVKQIPVKKGANFRDLKGVKVGANNVVEWDPEVERVYLSSGKPLVPDYAMSFIKGKSLKPFGRLWWDETVPTVVTRAEPHNQVILHPTQARVLTIRENARLQGFPDYYRLFGPIKEKYIQVGNAVAVPVARALGYCLGQAYLGESDGSQPLYQLPASFTSVGRTAVQANAVSVGTPAGEVVEQ

SEQ ID NO: 97 - DNA, 1291 bp, Oryza sativa
gcacgagcttacaatttttattaggctgtcattctaaggagaaattacccccttttccactgcctacgcatgaggcgattgtgaagaatggctgcccgttggcttttgagcgtaatttggttggttggcccaatgacacaccaatgcaactagcaagaccaattgtccttgaagacattctttctgatctcccagaagtggcaaatggggaaagccgtgatgaaatgctgtatgtaaagggtcctcaaactgaattccaaagatacattcggtcatttaatgttgaagtgcatggtcccagagctcatgttacaaaagattccaaatcctcaaaattgtatgatcatcgaccattggtactggataatgataactaccaaaggatattgcaaattcccaagagaaagggagctaattttagagacctttcaggagtcatagttggtcctgacaatgtggctagattagatccaacgaaagaaagagtacttttgccatctggtcgtcctctggccttgatacatcctgcacaagacagactgcttactattcgtgaaagtgctcggttgcaaggctttcctgacaattacaggtttcgtggaacagtcaaagacaggtatcgccagattggaaatgcggtggccgtacctgttggacgagcacttggatacgccctggcgatggcctacctgaagaagtctggagacgatccccttatgttgctgccgcccaactttgcattctcccatgacttaagagggtttgcctagtcaaacacccttgtcagaaggcacaaatttgagttccttccctcgttggtttgattgaaattcaccatctacattaatgctctgctacaaaagctgctcctcttcttgttgccaccctttctcgtgaatagtgaatagcattaggagttaggacatgccatttatctgttgggatcagatcaaggaatctgggttccccaatctgatgatcagctgtttgttgtagcaaaatcattccttgatttacagtagttgtgacatcccccacactagttaattggaaggattccatagggcaagtgtagtgactcatatcgggagcctatgttgtagtgtaaaagaatgtatgtacaagttagatgattaggaaaataaaatgtatggacatcatcttacctgttgttgtgttagccagcgatgaacacaaatgtaacgacttcaataaatctacattgagctgcagatttccaatgggcctaaaaaaaaaaaaaaaaagaaaaaaaaaaaaaaaaaaaaaaaaa

SEQ ID NO: 98 - protein, 255 aa, Oryza sativa
TSLQFLLGCHSKEKLPPFPLPTHEAIVKNGCPLAFERNLVGWPNDTPMQLARPIVLEDILSDLPEVANGESRDEMLYVKGPQTEFQRYIRSFNVEVHGPRAHVTKDSKSSKLYDHRPLVLDNDNYQRILQIPKRKGANFRDLSGVIVGPDNVARLDPTKERVLLPSGRPLALIHPAQDRLLTIRESARLQGFPDNYRFRGTVKDRYRQIGNAVAVPVGRALGYALAMAYLKKSGDDPLMLLPPNFAFSHDLRGFA

SEQ ID NO: 99 - DNA, 1324 bp, Glycine max
gcacgaggtcgttgatattttgaagttttctggtggttatctggggcgttatgccataggccgtcttgtagctatgaattatcaagcaagaatgggcatgatggcagcagggtcttatggccttccacaatttcgtatgcgtgttttcctttggggagctcgtcctactgagcaattgcctccatatccgttaccaacgcatgaggtggtgtcaagaggttttgttcccactgagtttgaagaaattacagtagcttatgataaaaaggatacctgtcagctggctggtgctcttttacttgatgatgcaatatcagatctcccacctgttacaaatgatgagaaccaggatgaaaggaactatggagctcctgctcgaactgagtttcaaagatatattagattgaagaaaaacgagatggtgggtagcatggctactgctcaaagcacaccacgtagaatactgtatgatcatcgtcctcttcaattgaacaaagatgattatgacagagtttgccagattccccagaagaagggtgcaaacttcagagatctacctggagtacttgtgaatggcaacaaagttgaatgggatccatcggttgaaagagtgatgctagactctggaaagcctttggttcctgattatgcaatgacatttgtccgtgggacctctactaagccatttggtcggttgtggtgggatgaaattgtgccaacagtggtgacaagggccgagcctcacaaccaggccattctccaccctagacaaaaccgagtacttaccattagagagaatgcgagactacaaggatttcctgattgctataaactttgtgggcctgtcaaagagaggtacatacaagttggaaatgctgttgctgttcctgttgctctagcattgggatacacgtttggtttggcctgccagggactgtctgacgataagcctttgacaaccctccccttcaagtatccaagttgtcttgccctttcatctcttgcaggaactgagaatgataacgaatcaagttgagattgttcccacacaatcccttctctctgccattgccatgttgaacttttccatatttattcaaattttccattttaatgtaagagcgagtgtacactagaaggccgaaaagttgtatcaatgatgattgtatgtacactaatgacattgtttataatgatataagattcactttgttaatggttttgcaaaatttaagttgtattaaaaacaaaatgtatttaagaattaattaaacttgagatcatttgatcaaagttcttgttataaaaaaaaaaaaaaaaaaaa

SEQ ID NO: 100 - protein, 344 aa, Glycine max
HEVVDILKFSGGYLGRYAIGRLVAMNYQARMGMMAAGSYGLPQFRMRVFLWGARPTEQLPPYPLPTHEVVSRGFVPTEFEEITVAYDKKDTCQLAGALLLDDAISDLPPVTNDENQDERNYGAPARTEFQRYIRLKKNEMVGSMATAQSTPRRILYDHRPLQLNKDDYDRVCQIPQKKGANFRDLPGVLVNGNKVEWDPSVERVMLDSGKPLVPDYAMTFVRGTSTKPFGRLWWDEIVPTVVTRAEPHNQAILHPRQNRVLTIRENARLQGFPDCYKLCGPVKERYIQVGNAVAVPVALALGYTFGLACQGLSDDKPLTTLPFKYPSCLALSSLAGTENDNESS

SEQ ID NO: 101 - DNA, 527 bp, Zea mays
  unsure (435)
  unsure (506)
cattgcatctcttctggagatgggataccaggtccggtttggaattctagaagcaggggcttttggtgttgcccagtccaggaaaagggcgtttatttgggctgctgcacctggagagatgcttcctgattggccagagccgatgcatgtgtttgctagccctgagctgaagataacactgcctgatggccaatactatgcagctgcaagaagcactgctggtggagcgcctttccgagcgattactgttagagatacaattggggatctgcctaaagtgggaaatggtgccagcaaactcacgcttgagtatggaggtgagcccgtgtcttggttccagaagaagataagagggaatatgatggtactgaatgggcacatatctaaggagatgaatgagctgaacctaataaggtgccaacacattccgaaacngccgggttgtgattggcatgacctaccggacgagaaggttaagctgtcaaatgggcagatggctgacctgntaccttggtgcctgcccaaca

SEQ ID NO: 102 - protein, 175 aa, Zea mays
  UNSURE (145)
  UNSURE (169)
IASLLEMGYQVRFGILEAGAFGVAQSRKRAFIWAAAPGEMLPDWPEPMHVFASPELKITLPDGQYYAAARSTAGGAPFRAITVRDTIGDLPKVGNGASKLTLEYGGEPVSWFQKKIRGNMMVLNGHISKEMNELNLIRCQHIPKXPGCDWHDLPDEKVKLSNGQMADLXPWCLPN

SEQ ID NO: 103 - DNA, 791 bp, Oryza sativa
gcacgaggctatgtcaaggggacaggctccttactagctacgtccaataacctcaaacgaatttccaaagaggaccttgaaatttcttcactgaaggagttaggcttacgatttttcacccctcgtgaggttgctaatctgcattcttttccatcgagtttccactttccgaatcacataagccttaggcaacagtatgctatgctgggcaatagcctgagtgtagcagttgtggggcctttgctgcgttatctgtttgctgagacatagtgatgttcaatcagttggcacatgatgctacacaggacaaacacattgaccttcctttgcccgtgttatcaccctaaagccaggttggttcgtgggtatgattcgtgtactccccgaagctgatggttgtggttcaaaccttgcaacccaattggagtgaatttcccacagtggtgcaacttcaacttcatagggcttggggacttgtcgattcatcggtgggaagaagacagcgtgcatgatggcccggcatgtatcggtgaaactctgacgcgaatcttacctagtagagtagagaagcttcctcagcattttccctttgccccaggcataagttttaataataatgttgaatgtttgaggtgttacagcatttgactcatgcgcattgtgaaacgaggttattatggtttagaactgaacttcaacttcatgagctcaccattcgactgtctcaaaaaaaaaaaaaaaaaaaaaaaaaacaaaaaaaaaataaaaatgggggcgccgtagccagtcga

SEQ ID NO: 104 - protein, 89 aa, Oryza sativa
ARGYVKGTGSLLATSNNLKRISKEDLEISSLKELGLRFFTPREVANLHSFPSSFHFPNHISLRQQYAMLGNSLSVAVVGPLLRYLFAET

SEQ ID NO: 105 - DNA, 533 bp, Glycine max
  unsure (402)
  unsure (423)
  unsure (433)
  unsure (447)
  unsure (508)
  unsure (514)
  unsure (530)
aaagctaatcatcctgaggcattggtgttcattaacaattgcaatgttattcttagggctgtaatggagaagtgtggggacacagatgattgtatctcaacatctgaggctgcagaattggctgcaaagcttgatgagaaggaaataagtagtttaccaatgcctggacaagttgatttcattaatggtggtcctccatgccagggtttctctgggatgaataggtttaaccagagcagttggagtaaagtccagtgtgagatgatattggcattcttatcctttgctgattatttccggccaaggtatttcttgttggagaatgtgaggaactttgtgtctttcaataaaggacagacattccgtttaactttggcttcacttcttgagatgggttatcangtgaggtttggtatccttgangctggagcanttggggtttctcantcaagaaaaagggcattcatatgggctgcttctcctgaagatgtgcttcccgaatgggcanaaccantgcaagtcctttcggnccc

SEQ ID NO: 106 - protein, 176 aa, Glycine max
  UNSURE (134)
  UNSURE (141)
  UNSURE (145)
  UNSURE (149)
  UNSURE (170)
  UNSURE (172)
KANHPEALVFINNCNVILRAVMEKCGDTDDCISTSEAAELAAKLDEKEISSLPMPGQVDFINGGPPCQGFSGMNRFNQSSWSKVQCEMILAFLSFADYFRPRYFLLENVRNFVSFNKGQTFRLTLASLLEMGYXVRFGILXAGAXGVSXSRKRAFIWAASPEDVLPEWAXPXQVLS

SEQ ID NO: 107 - DNA, 1343 bp, Triticum aestivum
ttcggcacgaggagtaaagttcagtgtgagatgattttagcattcctctcttttgcggagtattttcgtcccagattctttcttttagaaaatgtcaggaattttgtttccttcaacaaaggacagaccttccgactggcagttgcatctcttctggaaatgggataccaggttcgttttggaatcttagaagcaggtacttttggtgttgctcagtccaggaaaagggcattcatttgggctgctgctcctggagagattcttcctgattggccagaaccgatgcaagtgtttgctagccctgaactgaaaataacactgcctgatggcaaatactatgcagctgccaaaagcactgctggtggggcgcctttccgtgcaataactgttagagatacaattggggatttgccgaaagtggaaaatggtgcaagtaaactcatacttgagtatggaggtgagcctacctcttggtttcagaagaagatcagaggtagcactattgcattgaacgatcacatatctaaggagatgaatgaattaaatctcataagatgcaaacacattcccaaacgacctggttgtgactggcatgacctgccagatgagaaggtgaagctatcttctgggcaaatggtggacctgataccttggtgcttgcctaacaccgctaaaaggcacaatcagtggaagggtctgtatgggaggttagattgggagggcaatttccccacttctgtgactgatcctcagccgatgggcaaggttggcatgtgcttccatcctgaccaggacaggattatcacggtccgtgaatgtgcgcgatctcagggctttcctgacagctaccagttttcgggcaccattcagagcaagcacaggcagattggcaacgctgtgccaccccctcttgcctttgcgcttgggaggaagctgaaggaagccgtcgatgcgaagcgtcagcaggcctgacagcgtccgctagaactcttgagggacagaagcaccagcattcaagtttctcctgtattgtgcatggctggcatgaactgtcccgaaccagcattcaagccacatactgatgtgacgatgtgaactatcatgcatgaactttcttagtcgattgtgattttattggtctccttgtatatccttagattattgttgattggcgcccaaattcagagttgtcattgtatccaaacattgcacgagcgtacgtctactagtggctgttgcatgtggtgtgacatgttctggagctaagatataaaacgtttcctcgtactcgatatttggggatagaagtcttttgtgtgtatgaaaaaaaaaaaaaaaaaaaa

SEQ ID NO: 108 - protein, 323 aa, Triticum aestivum
FGTRSKVQCEMILAFLSFAEYFRPRFFLLENVRNFVSFNKGQTFRLAVASLLEMGYQVRFGILEAGTFGVAQSRKRAFIWAAAPGEILPDWPEPMQVFASPELKITLPDGKYYAAAKSTAGGAPFRAITVRDTIGDLPKVENGASKLILEYGGEPTSWFQKKIRGSTIALNDHISKEMNELNLIRCKHIPKRPGCDWHDLPDEKVKLSSGQMVDLIPWCLPNTAKRHNQWKGLYGRLDWEGNFPTSVTDPQPMGKVGMCFHPDQDRIITVRECARSQGFPDSYQFSGTIQSKHRQIGNAVPPPLAFALGRKLKEAVDAKRQQA

SEQ ID NO: 109 - DNA, 500 bp, Triticum aestivum
  unsure (438)
  unsure (500)
cagaagcctacgagaaccacagtccagccgaccagccgccgccgcgcgataaccggagttaatggagacgccgcccccatggagggtcctcgagttctacagcggtatcggcggcatgcggtactcccttgcgtcgtcgggcgttcgagcggaggtggtggaggcctttgacatcaacgacgtcgccaacgacgtctacgagcacaacttcggccaccgcccctgccagggaaacattcaaacactcactgctggtgatctagacaagtacaaggcacatgcatggctcctttctcctccatgtcaaccatatacacggacttcagaaacattcagctgatgctcgggcattttcatttataaagattttaaaccttatgcaaaacatgagctttcctccacaaatgttatttgtggaaaatgttgtccggattcgangtttttttaagcttaaaattattgaatccacagttgactgtagtattggactctacaatatn

SEQ ID NO: 110 - protein, 79 aa, Triticum aestivum
RVLEFYSGIGGMRYSLASSGVRAEVVEAFDINDVANDVYEHNFGHRPCQGNIQTLTAGDLDKYKAHAWLLSPPCQPYTR

SEQ ID NO: 111 - DNA, 791 bp, Oryza sativa; identical to SEQ ID NO: 103

SEQ ID NO: 112 - protein, 89 aa, Oryza sativa; identical to SEQ ID NO: 104

SEQ ID NO: 113 - DNA, 1492 bp, Glycine max
tttaaagctaatcatcctgaggcattggtgttcattaacaattgcaatgttattcttagggctgtaatggagaagtgtggggacacagatgattgtatctcaacatctgaggctgcagaattggctgcaaagcttgatgagaaggaaataagtagtttaccaatgcctggacaagttgatttcattaatggtggtcctccatgccagggtttctctgggatgaataggtttaaccagagcagttggagtaaagtccagtgtgagatgatattggcattcttatcctttgctgattatttccggccaaggtatttcttgttggagaatgtgaggaactttgtgtctttcaataaaggacagacattccgtttaactttggcttcacttcttgagatgggttatcaggtgaggtttggtatccttgaggctggagcatttggggtttctcagtcaagaaaaagggcattcatatgggctgcttctcctgaggatgtgcttcctgaatggccagaaccagtgcacgtcttttcggcccctgagttgaagatcacattatcagaaaatgtccagtatgctgctgtccgcagtactgcaaatggtgctcctttacgtgcaataactgttcgagatactattggtgatctcccagctgttggcaatggagcctcaaaaggaaacatggagtatcaaaatgatccagtctcatggtttcaaaagaagattcgaggtgatatggttgtcttgactgatcatatatcaaaggagatgaacgaattgaacttgattcgatgccagaaaattcccaagagaccaggtgctgattggcgtgaccttccagaagaaaagataaaactgtctagtggacaagttgttgatttgataccatggtgcttgccaaacacggctaagcggcacaatcaatggaagggactgtttggcaggttggattggcaaggaaatttcccaacttccgttacagaccctcagccaatggggaaggttggaatgtgcttccaccctgaccaagataggattcttactgttcgtgaatgtgctcggtctcaaggcttcccagatagctatgaatttgctggcaatatcatacacaagcaccggcagattggtaatgctgtgcctcctcctctagcatcagcattggggagaaagctcaaggaagcagtggacagtaagagctccacttagaagatgggccttctacattaggtcatgcttattgtattcataacagtcaccaagatattgcaaatcatcattccgggttccaaaaactagaaaccccttgtatatagtgatatccattggcccttttttgggggtaattcccttttttaactttccccacccaaggaattgaatgggtggtgccccttttttttcaactggggtttttttggtttaaaaaaaaaaaaaaaaaaaaaaaaaaaaaaaaaaaaaaaaaaaaaaaaaaaaaaaaaaaaaaaaaaaaaaaaaa

SEQ ID NO: 114 - protein, 400 aa, Glycine max
FKANHPEALVFINNCNVILRAVMEKCGDTDDCISTSEAAELAAKLDEKEISSLPMPGQVDFINGGPPCQGFSGMNRFNQSSWSKVQCEMILAFLSFADYFRPRYFLLENVRNFVSFNKGQTFRLTLASLLEMGYQVRFGILEAGAFGVSQSRKRAFIWAASPEDVLPEWPEPVHVFSAPELKITLSENVQYAAVRSTANGAPLRAITVRDTIGDLPAVGNGASKGNMEYQNDPVSWFQKKIRGDMVVLTDHISKEMNELNLIRCQKIPKRPGADWRDLPEEKIKLSSGQVVDLIPWCLPNTAKRHNQWKGLFGRLDWQGNFPTSVTDPQPMGKVGMCFHPDQDRILTVRECARSQGFPDSYEFAGNIIHKHRQIGNAVPPPLASALGRKLKEAVDSKSST

SEQ ID NO: 115 - DNA, 1343 bp, Triticum aestivum; identical to SEQ ID NO: 107

SEQ ID NO: 116 - protein, 323 aa, Triticum aestivum; identical to SEQ ID NO: 108

SEQ ID NO: 117 - DNA, 1285 bp, Triticum aestivum
gcacgagcagaagcctacgagaaccacagtccagccgaccagccgccgccgcgcgataaccggagttaatggagacgccgcccccatggagggtcctcgagttctacagcggtatcggcggcatgcggtactcccttgcgtcgtcgggcgttcgagcggaggtggtggaggcctttgacatcaacgacgtcgccaacgacgtctacgagcacaacttcggccaccgcccctgccagggaaacattcaaacactcactgctggtgatctagacaagtacaaggcacatgcatggctcctttctcctccatgtcaaccatatacacgacttcagaaacattcagctgatgctcgggcattttcatttataaagattttaaaccttatgcaaaacatgagctttcctccacaaatgttatttgtggaaaatgttgtcggattcgaggtatctgatacacatgaccagttgctagcggtcctttcaactctcagtttcaacacacaagaattcatcctaagccccttgcagtttggtgtcccatattctagaccgcgctacttctgtttggcaaaacaggaatctatgtgttttccaaatccatcagtcaatgacaagctgcttaggacacctacatgcctaacattgaacactacgagaactcagaatagctatgaccagaatgaagatgatctggaagtagtatgtaatccaataagaaacttccttgaagcacagagtattggagataaggaatcttcagccatcatctctgactttaaggaggctgatggatgcactccgattgaaactgcttcacatgactacacagttccactaagcttgattgaacggtggggaaatgctatggatattgtataccctgaatcaaaacggtgctgctgttttactaaaagttattatcgctatgtgaagggcacaggctctgtactggttacatctaaaagcctcaaaccagttccaaaagagaaccttgaaatgtcttcactgagtgagttgggtctacggtttttcacccctcgagaggtcgctaatttgcattcatttcccccgagtttccgttttccggatcagataagcctcagacaacagtatgccatgctgggtaatagtctgagcatagcggttgtggctcctttgttgtgctatctgtttgccgagacatagacaagttctcaatcatttggcaggtgatattcaccttggcccgccatctgatatttgtcaaaatcttgccacacttgagttgcaagtatgatattttatcc

SEQ ID NO: 118 - protein, 371 aa, Triticum aestivum
METPPPWRVLEFYSGIGGMRYSLASSGVRAEVVEAFDINDVANDVYEHNFGHRPCQGNIQTLTAGDLDKYKAHAWLLSPPCQPYTRLQKHSADARAFSFIKILNLMQNMSFPPQMLFVENVVGFEVSDTHDQLLAVLSTLSFNTQEFILSPLQFGVPYSRPRYFCLAKQESMCFPNPSVNDKLLRTPTCLTLNTTRTQNSYDQNEDDLEVVCNPIRNFLEAQSIGDKESSAIISDFKEADGCTPIETASHDYTVPLSLIERWGNAMDIVYPESKRCCCFTKSYYRYVKGTGSVLVTSKSLKPVPKENLEMSSLSELGLRFFTPREVANLHSFPPSFRFPDQISLRQQYAMLGNSLSIAVVAPLLCYLFAET

SEQ ID NO: 119 - DNA, 469 bp, Glycine max
attcggatctatctcacaccctttctctcttttcttcttttctttcgttttcccttcgcaagtgagagatggcgcaaattctgcttcatgggactctccacgccaccgtcttcgaggttgataggctcaatgctggtggtggtggcggcaattttttcagcaagctcaagcaaaactttgaggagactgttggcatcggaaagggagttactaaactctatgcaaccattgatctggagaaagcaagagtaggaaggactagaatcatagaaaatgaacatactaatcccagatggtatgagtcttttcacatttattgtgctcatatggcttcaaatatcatattcactgtgaaagatgataatcctattggggcaactttaattggaagagcttatgtgcctgtttcggaggtcttggatggtgaggaaatagataggtgggttgaaatcttggacgaggaaaaaaa

SEQ ID NO: 120 - protein, 133 aa, Glycine max
MAQILLHGTLHATVFEVDRLNAGGGGGNFFSKLKQNFEETVGIGKGVTKLYATIDLEKARVGRTRIIENEHTNPRWYESFHIYCAHMASNIIFTVKDDNPIGATLIGRAYVPVSEVLDGEEIDRWVEILDEEK

SEQ ID NO: 121 - DNA, 475 bp, Triticum aestivum
  unsure (349)
  unsure (426)
  unsure (429)
  unsure (461)
atctgcctccctcccgattcatcgtctcccgtcccctctttgtcgtcctcttcaccagcggcaggcggtggtggcaggtgactgtgactagcaggagcgcggaggaggggtccttcggtcgccatggctcagatcttgctccatgggaacctccacgtcaccatcttcgaggcctcctcgctctcccaccccggccgcgccagcggcggcgcccccaattcatccgcaatttgtagagggatttgaggaaactgtcggtgttggcaaaggaagtccaagctatatgccaccattgatctccgagaaactcgtgttgggcgtacaagatgttggggcaacgacccgtcancctccgctggtacgattcgttcacatctactgtgcgcacttgccgccgatgttatctcaagctgaaaggcgacaccnatntgggcgacctcatgggaaggcgtactccttcngaaactggaaggga

SEQ ID NO: 122 - protein, 94 aa, Triticum aestivum
  UNSURE (41)
  UNSURE (76)
MAQILLHGNLHVTIFEASSLSHPGRASGGAPNSSAICRGIXGNCRCWQRKSKLYATIDLRETRVGRTRCWGNDPSXSAGTIRSHLLCALAADVI

SEQ ID NO: 123 - DNA, 2736 bp, Glycine max
gcacgagcactcactcactcacctccatttccattgtcactgctccttattcggatctatctcacaccctttctctcttttcttcttttctttcgttttcccttcgcaagtgagagatggcgcaaattctgcttcatgggactctccacgccaccgtcttcgaggttgataggctcaatgctggtggtggtggcggcaattttttcagcaagctcaagcaaaactttgaggagactgttggcatcggaaagggagttactaaactctatgcaaccattgatctggagaaagcaagagtaggaaggactagaatcatagaaaatgaacatactaatcccagatggtatgagtcttttcacatttattgtgctcatatggcttcaaatatcatattcactgtgaaagatgataatcctattggggcaactttaattggaagagcttatgtgcctgtttcggaggtcttggatggtgaggaaatagataggtgggttgaaatcttggacgaggaaaaaaacccaatacaagagggttcaaagatccatgtgaagctgcaatattttgatgtcacaaaagaccgcaacagggctcgaggcattagaagtcccaaattccccggcgttccctatactttcttctcacagaggcaaggatgtaaggtatctctgtaccaagatgctcatgtacctgataattttgtacctaaaatacctcttgctggaggcaagaattatgaggctcataggtgttgggaggatatatttgatgcaatcactaatgctagacacttcatatacattactggttggtctgtttatactgaaatttccttggtgagggattctaggaggccaaagcctgggggagaccaaacacttggtgagcttctcaagaaaaaggcaaatgaaggggttaaggtattgatgcttgtttgggatgatagaacatcagttggtttgttgaaaaaagatggactaatggctactcacgatgaagaaactgcacagttctttgaaggcactgaggtgcattgtgttttatgcccccgcaatcctgatgatggtggtagcattgttcaggatttacaaatttctaccatgtttactcatcaccagaagatcgtggtggttgacggtgcgatgccaggtgaagggtcagataggcgaagaattgtgagttttgttgggggtattgacctctgcgacggaagatatgacactgccttccactcacttttcagaaccctagacacagcacaccatgacgattttcatcagcctaactttcctggtgctgctatcacaaaaggtggtcccagggaaccatggcacgacatccactcccggcttgaaggacccatagcttgggatgttttgttcaactttgagcagagatggagaaagcaaggtgggaaggatgtacttgttccactgagagagcttgaagatgtcattattcccccatccccagtaacgtttcctgaagatcatgagacctggaatgttcagttgtttagatccattgatggtggggctgcttttgggttcccggagactcctgaagatgctgccagagctggtcttattagtgggaaggataatatcattgatcgtagcattcaagatgcttatattaatgctattcgacgtgcaaagaacttcatctatattgaaaatcagtatttccttggaagctcttttgcctggagtgctgatgatattaagcctgaagacattggtgctttgcatctaatcccaaaggaactttcactcaagattgttagtaagattgaagctggggaaaggtttgctgtgtatgttgtagtcccaatgtggccagagggtgttccagaaagtgcatcagttcaggcaatattggactggcagaagagaacaatggagatgatgtacaaggacattattcaggcactcagagctaagggaatcgatgaagatcctcgaaactatttgacattcttctgccttggcaaccgggaagtgaagaaaccaggagaatatgagccttctgagcaaccagatcctgattcagattatcagagagcccaagaggcccgacgattcatgatttatgttcataccaagatgatgatagttgatgacgaatacataatcgttggatctgccaatatcaaccaaaggtcaatggatggtgctagggactctgagattgccatgggtgcttatcagccctatcatttggctaccaggcagccagcacgtggtcagattcacggtttccgcatgtcattgtggtatgagcaccttggcatgcttcatgactccttcctccagccagaaagtgatgaatgcattaacaaggtgaaccaagttgctgacaaatattgggatctgtattctaacgagtcacttgagcatgaccttcccggtcaccttctccgctaccccatcggggttgccagtgaaggagatgtcaccgagctgccaggatttgaattctttcccgacaccaaggctcgcattcttggtggcaaagctgactacctcccccctattctcactacttaatttgattttgtactttgttacaaacttgttattgcttacatcctaatctgtgttttcattactacttagtaataatcttaatatagtctgtgatgtcttgtggagatgttttttctgctatttttctgaagatttcagtgacagattttagttatctgaaaaaaaaaaaaaaaaaaaaaaaaaaaaaaaa

SEQ ID NO: 124 - protein, 809 aa, Glycine max
MAQILLHGTLHATVFEVDRLNAGGGGGNFFSKLKQNFEETVGIGKGVTKLYATIDLEKARVGRTRIIENEHTNPRWYESFHIYCAHMASNIIFTVKDDNPIGATLIGRAYVPVSEVLDGEEIDRWVEILDEEKNPIQEGSKIHVKLQYFDVTKDRNRARGIRSPKFPGVPYTFFSQRQGCKVSLYQDAHVPDNFVPKIPLAGGKNYEAHRCWEDIFDAITNARHFIYITGWSVYTEISLVRDSRRPKPGGDQTLGELLKKKANEGVKVLMLVWDDRTSVGLLKKDGLMATHDEETAQFFEGTEVHCVLCPRNPDDGGSIVQDLQISTMFTHHQKIVVVDGAMPGEGSDRRRIVSFVGGIDLCDGRYDTAFHSLFRTLDTAHHDDFHQPNFPGAAITKGGPREPWHDIHSRLEGPIAWDVLFNFEQRWRKQGGKDVLVPLRELEDVIIPPSPVTFPEDHETWNVQLFRSIDGGAAFGFPETPEDAARAGLISGKDNIIDRSIQDAYINAIRRAKNFIYIENQYFLGSSFAWSADDIKPEDIGALHLIPKELSLKIVSKIEAGERFAVYVVVPMWPEGVPESASVQAILDWQKRTMEMMYKDIIQALRAKGIDEDPRNYLTFFCLGNREVKKPGEYEPSEQPDPDSDYQRAQEARRFMIYVHTKMMIVDDEYIIVGSANINQRSMDGARDSEIAMGAYQPYHLATRQPARGQIHGFRMSLWYEHLGMLHDSFLQPESDECINKVNQVADKYWDLYSNESLEHDLPGHLLRYPIGVASEGDVTELPGFEFFPDTKARILGGKADYLPPILTT

SEQ ID NO: 125 - DNA, 2997 bp, Triticum aestivum
gcacgagatctgcctccctcccgattcatcgtctcccgtcccctctttgtcgtcctcttcaccagcggcaggcggtggtggcaggtgactgtgactagcaggagcgcggaggaggggtccttcggtcgccatggctcagatcttgctccatgggaacctccacgtcaccatcttcgaggcctcctcgctctcccaccccggccgcgccagcggcggcgcccccaagttcatccgcaagtttgtagagggcattgaggaaactgtcggtgttggcaaaggaagctccaagctatatgccaccattgatctcgagaaagctcgtgttgggcgtaccaggatgttgggcaacgagcccgtcaatcctcgctggtacgagtcgttccacatctactgtgcgcaccttgccgccgatgtgatcttcacgctgaaggccgacaacgcgatcggggcgacgctcattgggagggcgtacctgcctgtcggagagctcctggaaggggaggagatcgataggtggcttgaaatctgtgatgacaaccgggagcctgttggtgagagcaagatccatgtgaagcttcagtactttggcgttgagaaggaccgcaactgggcgaggggtgtccggagcgtcaagtttcctggtgttccttacaccttcttctcgcagaggcaaggatgcaatgttagattgtaccaagatgctcatgtcccagacaactttatccccaagattccgcttgcggacggcaagaactatgagcctgccagatgttgggaggatatctttgatgccataagcaatgctcagcatttgatttacatcactggttggtctgtgcacactgagatcaccttgattagggacaccaatcgccccaaacctggaggagacgtcactctcggagagttactcaagaggaaggccagcgaaggtgtccgggtccttatgctagtgtgggatgatagaacttcagttggcttgctgaagagagatggcttgatggccacccatgatgaggagactgcaaattacttccaaggcaccgatgtgcactgtgttctgtgccctcgtaaccccgatgattcaggcagcattgttcaggatctgcagatctcaaccatgttcactcaccatcagaagatagtatgtgttgacgatgcattgccaagccagggctccgagcaaaggaggatactcagcttcgttggtggcattgacctctgcgacggaagatatgacactcagtaccactccttgtttaggacacttgacactgtccaccatgatgacttccaccagcctaacttcgcgactgcatccatcaccaaaggtggcccaagagagccatggcatgatattcattcacgattggaaggtccaattgcctgggatgttctttacaattttgagcagagatggagaaagcagggtggcaaagatcttctcgtgcagctcagggatctctctgacataattatccccccttctcccgtcatgttcccagaggacagagatacatggaatgtccagctcttcagatctattgatggtggtgctgcttttggcttccctgatactcctgaggaagctgcaagggctgggcttgtaagtggaaaggatcaaatcattgacaggagcatccaggatgcatacataaatgccatccgacgggcaaaggacttcatctacattgagaaccaatacttccttggaagttcctactgctggaagcccgaaggcatcaagcctgaagaaattggcgctctgcatgtgattcctaaggagctttcgttgaagattgtcagcaagattgaagccggagaacggtttactgtttatgttgtggtgccaatgtggcctgagggcatgccagagagcgcatctgtacaagcaattctggactggcaaaggagaacaatggagatgatgtacactgacatcacacaagctctcgaggcaaaggggattgaagcaaaccccaaggaatacctcactttcttctgcctaggtaaccgtgaggtgaagcaggatggggaatatgaaccccaggagcagccagaacctgatactgattacgtccgcgctcaagaggctaggaggttcatgatctacgttcataccaaaatgatgatagttgatgacgagtacatcatcattgggtctgcaaacatcaaccaacggtcaatggacggcgcccgggactccgagattgccatgggcgcttaccagccataccatctagccaacagggagccggcccggggccagatccacggcttccggatggcactgtggtacgagcacctgggcatgctggacgacgtgttccagcgcccggagagcgtcgagtgcgtgcagaaggtgaacaggatcgcggagaagtactgggacatatactcgagcgacgacctggagcaggacctccccggccacctgctgagctaccccatcggcgtcgccagcgacggcgtggtgacggagctgccgggcatggagttcttccccgacacccgggcccgcatcctcggcgccaagtcggactaccttccccccatcctcaccacatagataagatctcatcctgcatttcctgtgtgtggcttcagtttggtgattcagaacttgtgtttcagaaaatagcaggctgttatagttgcgggactgttaataagcgcagtggtgtgcatggttgagaaccacggtagtgactaggaggattgctgatactttacacggtttctgctgtttgtatcactgtttaattataaattgtcaatgtttagttgtttacgctcgtcatacctacttggatctttaagggtggacaataaagttaacgcgtgtttcttattgtatgatggatatatagatagttttgtttttccaatgtatggtatcagagtgctgtatgtttggtttttccactgaataattaagctgtattaggctggtcaaaaaaaaaaaaaaaaaaaaaaaaaaaaaaaaaaaaaaaaaaa

SEQ ID NO: 126 - protein, 812 aa, Triticum aestivum
MAQILLHGNLHVTIFEASSLSHPGRASGGAPKFIRKFVEGIEETVGVGKGSSKLYATIDLEKARVGRTRMLGNEPVNPRWYESFHIYCAHLAADVIFTLKADNAIGATLIGRAYLPVGELLEGEEIDRWLEICDDNREPVGESKIHVKLQYFGVEKDRNWARGVRSVKFPGVPYTFFSQRQGCNVRLYQDAHVPDNFIPKIPLADGKNYEPARCWEDIFDAISNAQHLIYITGWSVHTEITLIRDTNRPKPGGDVTLGELLKRKASEGVRVLMLVWDDRTSVGLLKRDGLMATHDEETANYFQGTDVHCVLCPRNPDDSGSIVQDLQISTMFTHHQKIVCVDDALPSQGSEQRRILSFVGGIDLCDGRYDTQYHSLFRTLDTVHHDDFHQPNFATASITKGGPREPWHDIHSRLEGPIAWDVLYNFEQRWRKQGGKDLLVQLRDLSDIIIPPSPVMFPEDRDTWNVQLFRSIDGGAAFGFPDTPEEAARAGLVSGKDQIIDRSIQDAYINAIRRAKDFIYIENQYFLGSSYCWKPEGIKPEEIGALHVIPKELSLKIVSKIEAGERFTVYVVVPMWPEGMPESASVQAILDWQRRTMEMMYTDITQALEAKGIEANPKEYLTFFCLGNREVKQDGEYEPQEQPEPDTDYVRAQEARRFMIYVHTKMMIVDDEYIIIGSANINQRSMDGARDSEIAMGAYQPYHLANREPARGQIHGFRMALWYEHLGMLDDVFQRPESVECVQKVNRIAEKYWDIYSSDDLEQDLPGHLLSYPIGVASDGVVTELPGMEFFPDTRARILGAKSDYLPPILTT

SEQ ID NO: 127 - DNA, 387 bp, Zea mays
  unsure (289)
  unsure (291)
  unsure (307)
  unsure (311)
  unsure (385)
gcggacgcgtgggcgatcaattgattcgaattctgtcaagggttttccaaaagatccacggaaggccactagtaagaatcttgtttgtgggaaaaatgtactgattgatatgagcgtgcatacagcatatgtgaatgccatccgaggtgctcaacatttcatctatattgagaaccagtacttccttggctcttcatttaactgggattcacataaagatgttggtgctaataatctgatacccattgagatagcactgaaaattgcaaacaagatttattcgaatgananattttcagcttatatngtanttcccatgtggcctgaaggaaatcctaccagtactcctaccccaaagatctttattgattttccggttcaggangt

SEQ ID NO: 128 - protein, 116 aa, Zea mays
  UNSURE (92)..(93)
  UNSURE (98)
  UNSURE (100)
RSIDSNSVKGFPKDPRKATSKNLVCGKNVLIDMSVHTAYVNAIRGAQHFIYIENQYFLGSSFNWDSHKDVGANNLIPIEIALKIANKIYSNXXFSAYXVXPMWPEGNPTSTPTPKI

SEQ ID NO: 129 - DNA, 556 bp, Glycine max
  unsure (389)
  unsure (427)
  unsure (455)
  unsure (457)
  unsure (465)
  unsure (482)
  unsure (489)
  unsure (518)
  unsure (520)
acaccatggacaggctcagcaaattgttccatttcaaaccacttcgagctccttaaggatcctgctcttacatggcaacttagaaatatgggtcaacgaggccagaaaccttcccaacatggacatgttccacaagaaaacgggagaaatggtttccatgttgtcccgaaaacttggcggcaaaatcgaaggtcacatgtccaaagctggaaccagtgatccctatgttacggtatctgtggccggtgctgtgattgccagaacttttgtcatcagaaacagtgagaaccctgtttggacacagcatttcaatgtccccgttgcacatcttgcttctgaagttcactttgttgtcaaggacagtgatattgtggggttctcaagattantgggagcagttgggaattccagtgggaacatttagtantgggacaagagttgagggctttttcccnanccttgggngctaatgggaaaccaanttaaggntggttaaggttgagtttaccaatccaagnancccctgtttgaaaagggggcccctttaaacccatgg

SEQ ID NO: 130 - protein, 150 aa, Glycine max
  UNSURE (129)
  UNSURE (140)..(141)
  UNSURE (146)
HGQAQQIVPFQTTSSSLRILLLHGNLEIWVNEARNLPNMDMFHKKTGEMVSMLSRKLGGKIEGHMSKAGTSDPYVTVSVAGAVIARTFVIRNSENPVWTQHFNVPVAHLASEVHFVVKDSDIVGFSRLXGAVGNSSGNIXXWDKSXGLFP

SEQ ID NO: 131 - DNA, 866 bp, Zea mays
ccacgcgtccgcggacgcgtgggcgatcaattgattcgaattctgtcaagggttttccaaaagatccacggaaggccactagtaagaatcttgtttgtgggaaaaatgtactgattgatatgagcgtgcatacagcatatgtgaatgccatccgaggtgctcaacatttcatctatattgagaaccagtacttccttggctcttcatttaactgggattcacatagagatgttggtgctaataatctgatacccattgagatagcactgaaaattgcaaacaagatttattcgaatgagagattttcagcttatatagtagttccaatgtggcctgagggaaatcctaccagtactcctacgcaaaggattctttattgattttcgggtacaggatgtctctatgggtagagcaaataagggggacggtgcactccccccttcgtgtgtgttttaatcaccatttctaaaatttattctattgaagtgatgatgacagtccatcgccgccaagaggtcacaggagaagaactcgcagcccaagtcctcgtggtagttatggaggccatggcaagcgccccacaactaatcttatggtgacgaatcttggtccggattgtaggattccgactctcatcaagtgactcctcccctcacgatgactttgcggacagatgtctcaattatagtggtggagctacgaacaacaatccgatgtcctgatcgagagcgatggagctaatgtttgaacttgtgtgtttgaccttgtgaactaaaaatgtgaactatggatgtgaacttatatgaatttgtgaacttatgtatttggacttatgtgaatttgtgaacttaaaaaaaaaaaaaaag

SEQ ID NO: 132 - protein, 125 aa, Zea mays
TRPRTRGRSIDSNSVKGFPKDPRKATSKNLVCGKNVLIDMSVHTAYVNAIRGAQHFIYIENQYFLGSSFNWDSHRDVGANNLIPIEIALKIANKIYSNERFSAYIVVPMWPEGNPTSTPTQRILY

SEQ ID NO: 133 - DNA, 2797 bp, Glycine max
gcacgagacaccatggacaggctcagcaaattgttccatttcaaaccacttcgagctccttaaggatcctgctcttacatggcaacttagaaatatgggtcaacgaggccagaaaccttcccaacatggacatgttccacaagaaaacgggagaaatggtttccatgttgtcccgaaaacttggcggcaaaatcgaaggtcacatgtccaaagctggaaccagtgatccctatgttacggtatctgtggccggtgctgtgattgccagaacttttgtcatcagaaacagtgagaaccctgtttggacacagcatttcaatgtccccgttgcacatcttgcttctgaagttcactttgttgtcaaggacagtgatattgtgggttctcagattattggagcagtgggaattccagtggaacatttatgtagtggaacaagagttgagggctttttccccatccttggtgctaatgggaaaccatgtaagggtggatcagtgttgagtttatccattcagtacacccctgttgaaaaggtgcctctttatagccatggagtaggtgctggtcctgattatgaaggggtccctggcacctactttccccttagaaaaggtggaaaagttacactttatcaggatgctcatgttgaagaaggttgccttcccagtttgaaggtggatggatatgtgaattacaagcatggaagttgttggcatgacatatttgatgcaataagtgaggctcgtcgattggtttacattgtggggtggtctgtgtactacaatgttagtctcattcgggatagtgctaatggaaaatcctacactttaggtgatcttctcaaagccaaatcacaggaaggtgtgagagtgctgctccttgtttgggatgatcccacgtctaaaagcatgcttggatttaaaacggttggactcatgaacactcatgatgaggacactcgccagtttttcaagaactcttcagtacgagtgcttctttgcccacgagctggtggaaaaggacatagctgggtcaaaacgcaggaagctggaacaatctatacccatcatcagaagacagtcattgtggatgctgatgcaggtcagaataaaagaaaaatcaaagctttcatcggaggtcttgatttatgtgtgggccgatatgataccccaaaccattccatctttaggactttgcagacaacacacaaagatgactatcataatcctaactttgaggggccagttactggttgtccaagacaaccatggcatgatttgcattcccaagttgatggtccagcagcatatgacattctcaccaattttgaggagcgttggttaagggcattaaaaatgcatagatttcaaaagatgaaaagttcacatgatgattcattactgaaaattgatagaatccctgacattgttggcattgatgaagttccttgccagaatgaaaataaccgggagacttggcatgcccaggtcttccgttcaattgattctaattctgtgaaaggatttccaaaggaaccacaagatgctataagaaggaacttggtttgtggaaagaatgtactgatagacatgagcatacattcagcgtatgtcaaggcaattcgagcagcccaaaagtttatctatattgagaaccaatactttcttggctcgtcatataattgggattcttacaaagaccttggtgcaaacaacttaattccaatggaaattgcattaaaaatagccaataaaatcaaacaacatgagagattttctgtgtatattgtcattcctatgtggcctgaaggtgtacctacaagtacagctactcagaggattctcttttggcagttcaaaacaatgcaaatgatgtatgaaacaatttacaaggccctacaggaggctgggcttgacaataagtatgaaccacaggactacttgaatttcttttgccttggcaatcgtgagatacctgacaatgaaaatgttttaaatgatgtaaaaactactggagaaaacaagcctcaggcactcactaaaaagaaccggagattcatgatttatgttcattcaaaaggaatgatagtggatgatgaatatgtgttactggggtctgcaaacataaaccagcgatccatggaaggcaccagagatacagagatagcaatgggggcatatcagcctaatcatacttgggcaaagaagcaatctaaacctcacggacaggtgcatggttatagaatgtcactatggagcgaacatataggagccgtggaagaatgttttgaggaaccagagagccttgaatgtgtaagacggataaggtcattgagtgagtttaactggagacaatatgcagcagaagaggtaactgaaatgaaaagtcatctattaaaatatccgcttgaagttgattcaaagggcaaagtgaagcctctttttggctgtgaggcattcccagacgttggtgggaacataagtggcactttcacactactcaaagaaaatctcaccatctgatcatttacgtgagttctctaactcagtgaatactatagcagattttagtagcttatttacaatatttagattcttttggaaaagaaaagaaaaggagtataaagatttgaacttgtaacctgctttgaagtactagtttactcaatgtcttatttaagatttaaggtagtcttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 134 - protein, 840 aa, Glycine max
TRHHGQAQQIVPFQTTSSSLRILLLHGNLEIWVNEARNLPNMDMFHKKTGEMVSMLSRKLGGKIEGHMSKAGTSDPYVTVSVAGAVIARTFVIRNSENPVWTQHFNVPVAHLASEVHFVVKDSDIVGSQIIGAVGIPVEHLCSGTRVEGFFPILGANGKPCKGGSVLSLSIQYTPVEKVPLYSHGVGAGPDYEGVPGTYFPLRKGGKVTLYQDAHVEEGCLPSLKVDGYVNYKHGSCWHDIFDAISEARRLVYIVGWSVYYNVSLIRDSANGKSYTLGDLLKAKSQEGVRVLLLVWDDPTSKSMLGFKTVGLMNTHDEDTRQFFKNSSVRVLLCPRAGGKGHSWVKTQEAGTIYTHHQKTVIVDADAGQNKRKIKAFIGGLDLCVGRYDTPNHSIFRTLQTTHKDDYHNPNFEGPVTGCPRQPWHDLHSQVDGPAAYDILTNFEERWLRALKMHRFQKMKSSHDDSLLKIDRIPDIVGIDEVPCQNENNRETWHAQVFRSIDSNSVKGFPKEPQDAIRRNLVCGKNVLIDMSIHSAYVKAIRAAQKFIYIENQYFLGSSYNWDSYKDLGANNLIPMEIALKIANKIKQHERFSVYIVIPMWPEGVPTSTATQRILFWQFKTMQMMYETIYKALQEAGLDNKYEPQDYLNFFCLGNREIPDNENVLNDVKTTGENKPQALTKKNRRFMIYVHSKGMIVDDEYVLLGSANINQRSMEGTRDTEIAMGAYQPNHTWAKKQSKPHGQVHGYRMSLWSEHIGAVEECFEEPESLECVRRIRSLSEFNWRQYAAEEVTEMKSHLLKYPLEVDSKGKVKPLFGCEAFPDVGGNISGTFTLLKENLTI

SEQ ID NO: 135 - DNA, 462 bp, Zea mays
  unsure (4)
  unsure (31)
  unsure (105)
  unsure (412)
  unsure (436)
cggnacgctgggaggccacgctcccgtgccngctgcctcgtctgctccgcgcccatcaccaggctcatccgggaatataatgtacgagctaattctgttatgganaagacatactctattggacgatttgtcactggtttacctcctttctcgaaaaagaagaatgccgagaacaagtggtctctccataaggagggtctgcaaggccgtcagatagccgagaatatgcgggaaaaatacaacaagaaaccatggattttggaagacgaaacaggccaatatcagtatcaaggtcaaatggaaggatcacagtcagctacagctacatattatttgttaatgaagcacggcaaggaatttaatgcatatcctgctggctcttggtttaatttcagtaaaattgcacagtacnaacaattgacactggaggaggctnaagaaaagatgaataagaggaagacc

SEQ ID NO: 136 - protein, 83 aa, Zea mays
  UNSURE (69)
  UNSURE (77)
RQIAENMREKYNKKPWILEDETGQYQYQGQMEGSQSATATYYLLMKHGKEFNAYPAGSWFNFSKIAQYXQLTLEEAXEKMNKR

SEQ ID NO: 137 - DNA, 1710 bp, Zea mays
ccacgcgtccgcggacgcgtgggggcacgctcccgtgcccgctgcctcgtctgctccgcgcccatcaccaggctcatccgggaatataatgtacgagctaattctgttatggataagacatactctattggacgatttgtcactggtttacctcctttctcgaaaaagaagaatgccgagaacaagtggtctctccataaggagggtctgcaaggccgtcagatagccgagaatatgcgggaaaaatacaacaagaaaccatggattttggaagacgaaacaggccaatatcagtatcaaggtcaaatggaaggatcacagtcagctacagctacatattatttgttaatgaggcacggcaaggaatttaatgcatatcctgctggctcttggtttaatttcagtaaaattgcacagtacaaacaattgacactggaggaggctgaagaaaagatgaataagaggaagaccagtgcaactggttatgaacgctggatgatgaaagcagcggcaaatggaccagctgcctttggttcagacatgaagaagcttgaggccacaaatggtggggaaaaagaaagtgctcgtcctaagaaaggaaaaaataatgaagagggtaataattctgataagggtgaggaggatgaagaagaagaagcaacacgcaagaatagacttggactaactaaaaagggcatggatgatgatgaggaaggtgtaaaaaatctagatttcgatttggatgatgaaattgagaaaggtgatgactgggagcatgaagaaacattcactgacgatgatgaggctgtagacattgatccagaggaacgggcagatttagctcctgaaattcctgctccacctgaaatcaagcaggatgatgaagagaacgaagaagaagggggcctgagcaagtctggcaaggaactaaaaaagttgcttgggcgcgctgctggactaaatgagtcagatgcggatgaggatgaagaagacgaagatcaagaagatgattcatcgccagtgcttgctccaaaacagaaggatcaagttaaagatgaacctgtggatagcagcccatctaaaccagcaccatcaggacatgctcgaggcacacctccggcatccaaatccaagcaaaagagaaaatcaggtgttgatgatgcaaaaacttctagtggtgcagcttcaaagaaagcaaaggtggaattggatgcgaaagcatcaggcctcaaagaggaggcatcatctttggcaaaacctgcatcaaagacctctgctgcatcaaaaagtgggacaagcgtatcacctgtcacagaggatgaaatcaggagtgttcttcttgcagtggctccagtcaccacacaagatctggtatccagattcaagtctaggcttcgaggtgcagaggacaagaatgcatttgctgaaattctgaagaaaatttcgaagatacagaagacgaacggccacaactacgttgtcctcagagatgacaagaagtaaataaccgaacaaaggcgctgacatcgcaaccgttggctgtactgagataatatgtttatgtaacatgctgccttgaagataaacaagtctgtaacactagaattgtcttctaacccttgtgtacttgaattttattttactattccagattttatttatgcggctttgcactacacacctgttataaatggaaatccattgggagtt

SEQ ID NO: 138 - protein, 500 aa, Zea mays
PRVRGRVGARSRARCLVCSAPITRLIREYNVRANSVMDKTYSIGRFVTGLPPFSKKKNAENKWSLHKEGLQGRQIAENMREKYNKKPWILEDETGQYQYQGQMEGSQSATATYYLLMRHGKEFNAYPAGSWFNFSKIAQYKQLTLEEAEEKMNKRKTSATGYERWMMKAAANGPAAFGSDMKKLEATNGGEKESARPKKGKNNEEGNNSDKGEEDEEEEATRKNRLGLTKKGMDDDEEGVKNLDFDLDDEIEKGDDWEHEETFTDDDEAVDIDPEERADLAPEIPAPPEIKQDDEENEEEGGLSKSGKELKKLLGRAAGLNESDADEDEEDEDQEDDSSPVLAPKQKDQVKDEPVDSSPSKPAPSGHARGTPPASKSKQKRKSGVDDAKTSSGAASKKAKVELDAKASGLKEEASSLAKPASKTSAASKSGTSVSPVTEDEIRSVLLAVAPVTTQDLVSRFKSRLRGAEDKNAFAEILKKISKIQKTNGHNYVVLRDDKK

SEQ ID NO: 139 - DNA, 396 bp, Zea mays
gaacaagcaccagcgaaaccatccgatgtcaaaagaactcgtagggatcgtacggagatggaaaacattatattcaagctttttgaaaggcaacccaattgggcactaaaggcgctggtgcaagaaactgaccagccagagcaatttctgaaggagatattgaacgatctctgtgtgtataataaacgaggaccaaaccagggaactcatgagcttaagccagagtacaagaaatccacaggggacactgatgctgcttaaagatgatagacgacttggttgtttatgtgcagaatcgctagttctctgtcggtaactcggggcacacaaacacaagcgtgatctagtttatttgtatgctttaagaggtgttatttgtaagctggatacacattt

SEQ ID NO: 140 - protein, 86 aa, Zea mays
EQAPAKPSDVKRTRRDRTEMENIIFKLFERQPNWALKALVQETDQPEQFLKEILNDLCVYNKRGPNQGTHELKPEYKKSTGDTDAA

SEQ ID NO: 141 - DNA, 567 bp, Triticum aestivum
  unsure (361)
  unsure (495)
  unsure (515)
  unsure (526)
  unsure (531)
  unsure (554)
tagggatcgccgggaactggaaaacattatcttcaagcttttcgaaaagcagcctaactgggcactgaaggctctagtgcaagagactgaccagccagagcaattcctcaaggagatattgaacgacctctgtatgtacaacaaacgagggccaaaccagggcacgcacgagctcaagcccgagtacaagaagtcgtctgaggacgctgccggtgccccgtgaagatgatctatctgttttggcggctgttgtgtaactgtgataccaggaacctgttggcttgatgcgcggaactatgagttgtctatgtgaagcctccagtcatgcttggggctgcatttgcttgctttttattaagangcattgttgtaattgtatgggtataagatgatacatatacaacacatgtcacagagaggaaatggatctactgataaacacacgaaagattgtttgatttctgggtagatgtaagaatcattcaaggatcgacntgtgcatcctgtcctttganaatccgtactnaatanatcaaattttggctaaaatatangaatcacctgatt

SEQ ID NO: 142 - protein, 68 aa, Triticum aestivum
RRELENIIFKLFEKQPNWALKALVQETDQPEQFLKEILNDLCMYNKRGPNQGTHELKPEYKKSSEDAA

SEQ ID NO: 143 - DNA, 813 bp, Zea mays
agcccacgctccagttcaagatggagttggcacaaactaacactgggaatacacctaagagctactctttgaatatgttcaaagatttcgtgcctatgtgtgttttctccgaatctaaccaagggaaactttcatgcgaaggaaaagtcgagcataaatttgacatggaacctcacagtgataatttggcgaactatggaaagttatgtcgtgaaagaacacaaaaatatatggttaaatctagacaagtgcaggtacttgacaatgaccacggtatgagcatgagaccaatgcctggcttggttggtctcataccttctggttcccacgcgtccgaacaagcaccagcgaaaccatccgatgtcaaaagaactcgtagggatcgtacggagatggaaaacattatattcaagctttttgaaaggcaacccaattgggcactaaaggcgctggtgcaagaaactgaccagccagagcaatttctgaaggagatattgaacgatctctgtgtgtataataaacgaggaccaaaccagggaactcatgagcttaagccagagtacaagaaatccacaggggacactgatgctgcttaaagatgatagacgacttggttgtttatgtgcagaatcgctagttctctgtcggtaactcgggcacacaaacacaagcgtgatctagttttatttgtatgctttaagaggtgttatttgtaagctggatacacatttgtttagagaggaaaaggggaaagataattgaaacaaaatgtaagagtctaaattgttggaccaatgttctttatcgggtatt

SEQ ID NO: 144 - protein, 197 aa, Zea mays
PTLQFKMELAQTNTGNTPKSYSLNMFKDFVPMCVFSESNQGKLSCEGKVEHKFDMEPHSDNLANYGKLCRERTQKYMVKSRQVQVLDNDHGMSMRPMPGLVGLIPSGSHASEQAPAKPSDVKRTRRDRTEMENIIFKLFERQPNWALKALVQETDQPEQFLKEILNDLCVYNKRGPNQGTHELKPEYKKSTGDTDAA

SEQ ID NO: 145 - DNA, 677 bp, Oryza sativa
gcacgagggtacttgcgaatgacaatggaatgagcatgaggccgttgcctggcttggtgggtctgatgtcttctggtccaaaacagaaggagaagaagccactaccagtaaaaccatcagacatgaaaagaacgagaagggatcgcagggaactggaaaatatcttattcaagctttttgagaggcagccgaattggtctcttaagaatctcatgcaagaaactgatcaaccagagcaattcttgaaggagatattgaatgatctgtgtttctacaacaaaaggggtccaaatcaagggacgcatgaacttaagcctgaatacaagaaatctacagaggacgctgatgctactgctacttagaagtcttatgcttcggtctactagataggagtcctcgtccagtgtggactccaatccgttacttgctgcgttcaaggaaatcttggatacttcgtttatagtttgtttcttgagaaacatattttgtacgcatcaagcgatacatcttttggtgttactggcggccatatcttagacccagatttcggggactcgatatattcgtgtccatcagagtttagaaacaaggatgttgaaatttgtgtgtataatgtattttagctcttctaggcaaactaacatgatgattttcatgatcaaaaaaaaaaaaaaaaa

SEQ ID NO: 146 - protein, 119 aa, Oryza sativa
TRVLANDNGMSMRPLPGLVGLMSSGPKQKEKKPLPVKPSDMKRTRRDRRELENILFKLFERQPNWSLKNLMQETDQPEQFLKEILNDLCFYNKRGPNQGTHELKPEYKKSTEDADATAT

SEQ ID NO: 147 - DNA, 1365 bp, Oryza sativa
gaattcggcacgagcttacagattattgttcgttcatctaactgcacgtcacgccctccgcgtgtgcctccccttcgcccgctcgctccgtcgttaagccagatcgcccgccgctccgtgccctagcgccgccgccgccgtcgccgacaccggaatcgccgggcagactgcgagcgcggagccaccagcgtggggcggcgggatgggcgaggaggccaagtacctcgagacggcgcgggccgagcgctccgtgtggctgatgaagtgccccccggtcgtctcgcacgcctggcagggcgccgtgtcctcctccgacgccgccggctccaaccctaaccccgtcgtcgccaaggtcgtcctctcccttgacctcctccgctccgaggagccctccctccagttcaagatggagatggctcaaactaacactggcaatacaccaaagagttactccttgaatatgtccaaggattttgtaccaatgtgtgttttctctgagtctaaccaagggaaactttcatgtgaaggaaaagtcgagcataaatttgacatgaagcctcacagtgataatttggtgaactatggaaagttatgccgtgaaaggacacaaaagtccatgatcaaaactagaaaagtgcaggtaattgaggatcacagaatgagcctgataccgttgcctggcatggttggtctcataccttctggttctaaggagaagaagaagcaaacaccaaccaaaccatctgatgcaaaaagaatacgtagggatcgcagggaactggaaaatattatattcaagctttttgaaagacagcccaattgggcactaaaggcgctggtgcaagaaactgaccagccagagcaattcctgaaggagattctgaatgatctgtgtttttacaacaaacgaggaccaaaccagggaacgcatgagctcaagcctgagtacaagaaatctacaggggacactgatgcttcttgaatatgctagttcatttaactgctttcggataaccaacatactcaacctgttgactctgaagtcacgaacattgagttgtgcagacatctcatatttcagtaacattcgttctgctgctaaatgctaagaggcataggcgaatagttgtaaggatagtttgatacatttattagagaaaagaatagaaaagaaaaatctggaatctatgttcaggtaaaataatgtgcctggtgggagtcgcagcgtagagcatagtcctattcataattttcgttgtttatatgctgtttttatgtctcatttcatcatttgaaatgcaggatttatctgattctttgaagatgttttttttattaccaacaatattatgtcttcatattctt

SEQ ID NO: 148 - protein, 259 aa, Oryza sativa
MGEEAKYLETARAERSVWLMKCPPVVSHAWQGAVSSSDAAGSNPNPVVAKVVLSLDLLRSEEPSLQFKMEMAQTNTGNTPKSYSLNMSKDFVPMCVFSESNQGKLSCEGKVEHKFDMKPHSDNLVNYGKLCRERTQKSMIKTRKVQVIEDHRMSLIPLPGMVGLIPSGSKEKKKQTPTKPSDAKRIRRDRRELENIIFKLFERQPNWALKALVQETDQPEQFLKEILNDLCFYNKRGPNQGTHELKPEYKKSTGDTDAS

SEQ ID NO: 149 - DNA, 1286 bp, Oryza sativa
gaattcggcacgaggttctaacaatttctccgccttctcgtcctcctcgccgaaccacccccatcgacccacacaaccagcgaaccggaaggatttcactcagctgagtttcctgcctccatccccacggcgatggcggaggaggcgaagaatctggagacggcccgggccgaccgctccgtgtggctcatgaagtgcccgaccgtcgtctcccgcgcctggcaggaggccgccaccgccgccgcctcctcctcctcctcctcggacgccgccgctggcgccaactccaactccaacgcgaaccctaaccccgtcgtcgccaaggtcatcgtctccctcgacccgctccgctccgaagaccagcagctccagttcaagatggagatggctcaaacaggcaatggcaatacaccaaagagttactccttgaacatgttcaaggattttgtgccaatgtgtgtcttttctgaatccaaccaagggaagctttcatgcgaagggaaagtcgggcacaaatttgacatggaaccgcacagcgataatcttgtgaactatgggaagttatgtcgtgagaggactcaaaaatctatgattaaaaatagaaaattgatggtacttgcgaatgacaatggaatgagcatgaggccgttgcctggcttggtgggtctgatgtcttctggtccacaacagaaggagaagaagccactaccagtaaaaccatcagacatgaaaagaacgagaagggatcgcagggaactggaaaatatcttattcaagctttttgagaggcagccgaattggtctcttaagaatctcatgcaagaaactgatcaaccagagcaattcttgaaggagatattgaatgatctgtgtttctacaacaaaaggggtccaaatcaagggacgcatgaacttaagcctgaatacaagaaatctacagaggacgctgatgctactgctacttagaagtcttatgcttcggtctactagataggagtcctcgtccagtgtggactccaatccgttacttgctgcgttcaaggaaatcttggatacttcgtttatagtttgtttcttgagaaacatattttgtacgcatcaagcgatacatcttttggtgttactggcggccatatcttagacccagatttcggggactcgatatattcgtgtccatcagagtttagaaacaaggatgttgaaatttgtgtgtataatgtattttagctcttctaggcaaactaacatgatgattttcatgatcattaaacaccttgacctactct

SEQ ID NO: 150 - protein, 277 aa, Oryza sativa
MAEEAKNLETARADRSVWLMKCPTVVSRAWQEAATAAASSSSSSDAAAGANSNSNANPNPVVAKVIVSLDPLRSEDQQLQFKMEMAQTGNGNTPKSYSLNMFKDFVPMCVFSESNQGKLSCEGKVGHKFDMEPHSDNLVNYGKLCRERTQKSMIKNRKLMVLANDNGMSMRPLPGLVGLMSSGPQQKEKKPLPVKPSDMKRTRRDRRELENILFKLFERQPNWSLKNLMQETDQPEQFLKEILNDLCFYNKRGPNQGTHELKPEYKKSTEDADATAT

SEQ ID NO: 151 - DNA, 642 bp, Triticum aestivum
caaaaggacacgtagggatcgccgggaactggaaaacattatcttcaagcttttcgaaaagcagcctaactgggcactgaaggctctagtgcaagagactgaccagccagagcaattcctcaaggagatattgaacgacctctgtatgtacaacaaacgagggccaaaccagggcacgcacgagctcaagcccgagtacaagaagtcgtctgaggacgctgccggtgccccgtgaagatgatctatctgttttggcggctgttgtgtaactgtgataccaggaacctgttggcttgatgcgcggaactatgagttgtctatgtgaagcctccagtcatgcttgggctgcatttgcttgctttttattaagaggcattgttgtaattgtatgggtataagatgatacatatacaacacatgtcacagagaggaaatggatctactgataaaccacacgaaagattgtttgattttctgggtagatgtaagaatcatgtcgaggattcgacgtgtgcgatccttgttccttttgagaactcccgtacttcaatacaatccagatgtttgggctaaaacatatatggaatctagcctgatttaaaaaaaaaaaaaaaaaaaaaaaaaaaaaaaaaaaaaaaaaaa

SEQ ID NO: 152 - protein, 77 aa, Triticum aestivum
KRTRRDRRELENIIFKLFEKQPNWALKALVQETDQPEQFLKEILNDLCMYNKRGPNQGTHELKPEYKKSSEDAAGAP

SEQ ID NO: 153 - DNA, 2006 bp, Zea mays
caaatatttgcaagccacaagccaagccaagcaaaaccctaagaaacccaccctccctccagtctcctcgacccccgccgccgccgccgccatcacaacccagagcaccccctccccaccccaccctgtcccgatggctgcggacgccgccgctgcggcgatgtccgcgacttccctctgcgacgatctcgagcccgccaccgtgcgcacccgcatccgcgatgtcctggccgcaggtgccgcgcgtgccggggaccgcgtcgtggtcggtgggtgggtccggacgggccgggagcaggggaagggctctttcgccttcctggagctcagcgacgggtcctgcgctgccacgcttcaggccatcgttgacgccgccgtgcacccgctagcgcgcctcaccgccaccggcacctccgtcctcgtcgagggcgtgatcaaggagccgcccgaggggaccaagcagaacgtcgagctgaaggtcagccgcgtgcttgaggtcggcgaggtcgatgccgccgtctacccgctgcccaagggcaaggtcaaactcacgctcgagaagctcagggacgtcgttcatctccgttcgcgcaccaacacgattggagcggttgctaggattaggcaccagttagcctatgcatcacacaggttctttgatgagaatggatttctctatgtacatacaccaataataactacgagtgactgtgaaggtgctggtgagatgtttcaagtgacaactttatttagccaggctgagaaaactgagaaggaattgagagagaacccaaaaccttctgattctgaaattgaggcagctaaagttcttgtcaaggaaaaaggtgatgtggtcgcacagcttaaagctgcaaaagccagcaaacaagagatatcaactgctgtcgatgagcttaatagggcaaaggaaattgtttcaaaactggaggagaggttcaagttaaagcctgggattccacgcaaggatgatggttcaatagcttttgaaaatgacttcttcaaacgtcaagcttttttgacagtgtctggacagcttcaggttgagacatatgcatgtgctcttagtagcgtttatacctttggaccaacattccgggcagagaactcacatacgtcacgacatttggcagagttttggatggttgaaccggaaatcgcatttgcaaacttgcaggatgacatgaactgtgcagaaaaatatgtacagtacctttgcaagtggctgcttgaccattgccaggaagatatggagtttatggtgaaaaattatgataagagtgcaattgaacgcctggagcttgtttcgtcaactccttttgtgcggatttcatacacaaaggctgtggagctcttgaaaaatgttaccgacaagaagtttgacaacaaagttgaatggggaattgatttagcctctgagcatgaaaggtatttaacagaggatatatttaagaagccagttattgtctataactatccgaaaggaataaaggcattttatatgagactcaatgatgatgacaagacggtggctgcaatggatgttcttgtgcctaaggttggtgaattgattggtggaagccaaagggaggaacggttagatgttctgaaacagaggatacttgatgctggcctgcctttggaaccctatgaatggtacttggacctccgtcgctttggatctgtaaagcacagtggttttgggttgggttttgaaaggatgattctatttgcaaccggaatggagaacatcagagatgttatacccttcccaaggtacccaggaagggctgatctgtgatctttggagacattggttaaacagatgcgagttgaattcaacacgcgactagtgattttgatatgttttcaccgttaagtttctacttcctaatttgcacattttaattataaattgtggaactggtacggttgtcaaaaatcatgtaattttctggttgtattttattatatttgtatctctgatc

SEQ ID NO: 154 - protein, 561 aa, Zea mays
MAADAAAAAMSATSLCDDLEPATVRTRIRDVLAAGAARAGDRVVVGGWVRTGREQGKGSFAFLELSDGSCAATLQAIVDAAVHPLARLTATGTSVLVEGVIKEPPEGTKQNVELKVSRVLEVGEVDAAVYPLPKGKVKLTLEKLRDVVHLRSRTNTIGAVARIRHQLAYASHRFFDENGFLYVHTPIITTSDCEGAGEMFQVTTLFSQAEKTEKELRENPKPSDSEIEAAKVLVKEKGDVVAQLKAAKASKQEISTAVDELNRAKEIVSKLEERFKLKPGIPRKDDGSIAFENDFFKRQAFLTVSGQLQVETYACALSSVYTFGPTFRAENSHTSRHLAEFWMVEPEIAFANLQDDMNCAEKYVQYLCKWLLDHCQEDMEFMVKNYDKSAIERLELVSSTPFVRISYTKAVELLKNVTDKKFDNKVEWGIDLASEHERYLTEDIFKKPVIVYNYPKGIKAFYMRLNDDDKTVAAMDVLVPKVGELIGGSQREERLDVLKQRILDAGLPLEPYEWYLDLRRFGSVKHSGFGLGFERMILFATGMENIRDVIPFPRYPGRADL

SEQ ID NO: 155 - DNA, 1105 bp, Oryza sativa
gcacgagcttacacaggatttcttctgcaaaccagcatttctgacagtgtctgggcaactgaatggtgaaacatacgctacagctctatcagatgtttacacttttggtccaacatttagagctgaaaattcaaacacctcaagacatttggctgaattttggatgattgagcctgaacttgcctttgcggatctaaatgatgacatggcatgtgcgagttcatatctccagtatgtagtgaagtatgttctagagaactgcaaagaagatatggatttctttaatacatggattgaaaaagggatcatcgatagattaaatgacgtagttgagaaaaactttgttcacttgtcatattctgatgctattgagctacttgttgggtccaagaagaaatttgagttcccggtcaaatggggattggatctgcaaagtgagcatgaaagatatatcacagaagttgcttttggtgggcgcccggtgataattagagattatccaaaggaaatcaaagctttctatatgcgagagaatgatgatggtaaaacagttgctgcaatggatctattggttcctcgggttggtgaactcattggtggaagccaaagggaagaacgtcttgattaccttgaagctcgcttggatgagttaaatcttaacaaagatagctactggtggtacttagatctacgtcgatatggatcagttcctcatgctggttttggtcttggatttgaaagactagtacagtttgcaactggaatggacaacattagagacaccattccatttccccgggttccaggctctgcagagttttagaatccaacattgctcacaaatactttttgcaacattctttcctgagattgggatttcaagaaaagtatacactctactgtttcattgtacagagtttagttaagttattgcttacatcgagcaaaacaatattgttccaaattttgttgtttggagtaacgtgcttgcaaatttcattgaacttccgatgtacattattgttactggacgtgttataatgatcacctgatggattgatataaaaaaaaaaaaaaaaaaaaaaaaaaaaaaaaaaaaaaaaaa

SEQ ID NO: 156 - protein, 273 aa, Oryza sativa
HELTQDFFCKPAFLTVSGQLNGETYATALSDVYTFGPTFRAENSNTSRHLAEFWMIEPELAFADLNDDMACASSYLQYVVKYVLENCKEDMDFFNTWIEKGIIDRLNDVVEKNFVHLSYSDAIELLVGSKKKFEFPVKWGLDLQSEHERYITEVAFGGRPVIIRDYPKEIKAFYMRENDDGKTVAAMDLLVPRVGELIGGSQREERLDYLEARLDELNLNKDSYWWYLDLRRYGSVPHAGFGLGFERLVQFATGMDNIRDTIPFPRVPGSAEF

SEQ ID NO: 157 - DNA, 1849 bp, Glycine max
gcacgagagcaccaagaagcaaatggccgttagcattggcgttggcgccacaaaagcgatatcaatggcaatagcagcgaggcacctcggaacaaaaccttacgcagcagcagcaacaacaacggcacttgctcttctctcactccacaaacctcttttcctccctcattcctctcccttctcctctcgccgcgctttctgcgccgcgactcttcgcaccgccgataacagagtgcaacagttccgcaggaagcttagggtttccgaaatcaaagaaggcgacggcgccgacgtgttcggccgcaacctcgtcgtgcagggctgggtccgcacgctacgcattcagagtatcgtcaccttcctcgagattaacgacggttcttgcctttctaacatgcaatgtgtgttgaattcggaggctgaaggttacgatcaggtagaatctggcttggttaccacgggtgcttcagtgtgggtgcaaggagttgtggtgaagagtcaaggatcgaaacagaaggttgaattgaaggtcaacaaaatagtactgattggcaagagcgatccctcctttcccatccaaaagaaaagagccagcagagaatttctaagaacaaaagcacatcttcgtgcgaggacaaatacttttggtgcagttgcgagggttaggaatgcattggcatatgctacacataagttcttccaagaaaatgggtttgtatgggtctccagtcctatcatcacagcatcagattgcgagggagcgggtgaacagttttgcgttactaccttgataccaagttctcatgaaactactgattctcctgttgatgctattccaaaaacaaatgacggattaattgactggtcacaagatttctttggaaaacccgcattcttgactgtttcaggtcaactcaatggtgaaacttatgctacttctctctctgatgtgtatacatttggtcccacattccgagcagaaaattctaacacttctaggcacttggctgaattttggatgattgaaccggagcttgcatttgctgatctaaatgatgacatggcttgtgcaactgcttatctccagtttgtaataagacatgttctcgataattgcaaggaagacatggagtttttcgatacatggattaataaaggaatcattgatcgcttgagtgatgtagcagataaagatgttgtgcaaataacctacactgaagcgatagatctgctgtcaggagcaaataagaaatttgaattcccggtgaaatggggtagtgatcttcagagtgaacatgagcgttatataactgaagaggcattcagtggatgcccagtgataattagagactatccaaaggatattaaagcattctatatgcgacagaatgatgatggacggacagttgcagccatggacatgttggttccagggattggtgaacttattggtggaagccaaagagaagaaaggcttgagtatctagaagctcgtttagatgatttaaagctgaataaggatgcatattggtggtatcttgatttgcgtcgatatggttcagtacctcatgcaggctttggtttggggtttgaaagacttgtccaatttgcaacaggaatggacaatataagggacgttattccttttcctcgaacacctggctcggctgagttttagaccttgcagatataagcacttcacttcacattcatttgtacgattcaaactgtattttaggcaataaagtatcttgagtctttccttttcttctttttgttttgaaaaagaaaactgcttgcttataaatgaagtatttgtactttatgaatttagtagaatggaattctg

SEQ ID NO: 158 - protein, 551 aa, Glycine max
MAVSIGVGATKAISMAIAARHLGTKPYAAAATTTALALLSLHKPLFLPHSSPFSSRRAFCAATLRTADNRVQQFRRKLRVSEIKEGDGADVFGRNLVVQGWVRTLRIQSIVTFLEINDGSCLSNMQCVLNSEAEGYDQVESGLVTTGASVWVQGVVVKSQGSKQKVELKVNKIVLIGKSDPSFPIQKKRASREFLRTKAHLRARTNTFGAVARVRNALAYATHKFFQENGFVWVSSPIITASDCEGAGEQFCVTTLIPSSHETTDSPVDAIPKTNDGLIDWSQDFFGKPAFLTVSGQLNGETYATSLSDVYTFGPTFRAENSNTSRHLAEFWMIEPELAFADLNDDMACATAYLQFVIRHVLDNCKEDMEFFDTWINKGIIDRLSDVADKDVVQITYTEAIDLLSGANKKFEFPVKWGSDLQSEHERYITEEAFSGCPVIIRDYPKDIKAFYMRQNDDGRTVAAMDMLVPGIGELIGGSQREERLEYLEARLDDLKLNKDAYWWYLDLRRYGSVPHAGFGLGFERLVQFATGMDNIRDVIPFPRTPGSAEF

SEQ ID NO: 159 - DNA, 372 bp, Triticum aestivum
gcacgagtcttaacaaagatagctactggtggtatttggatctgcggcgatatggatcagttcctcatgctggttttggccttggatttgaacggcttgtacagtttgcaaccggaatagacaacatcagagacgccattccatttcccagggttcctggttctgcggagttttagcacttgaagagtgcgtgtataatctctaaaagcattcttgcctcggacttgaagaaaaccttacattgatttgttcattgtaaagagtttaccctttgcaaattcgtcgaatgctgggattttagtcgatgaattctttgtgtacgtccccattcagtaaacagacaattttgccttggggactaaaaaaaaaaaa

SEQ ID NO: 160 - protein, 57 aa, Triticum aestivum
TSLNKDSYWWYLDLRRYGSVPHAGFGLGFERLVQFATGIDNIRDAIPFPRVPGSAEF

SEQ ID NO: 161 - DNA, 573 bp, Triticum aestivum
  unsure (300)
  unsure (434)
  unsure (476)
  unsure (484)
  unsure (522)
  unsure (525)
  unsure (527)
  unsure (540)
  unsure (548)
  unsure (551)
ccgccgcctacccgctgcccaagaccaagatcacgctcgagacgctcagggacttcgtccacctccgcgcacgcaccaacacgataggcgcagttgctcggataaggcaccagcttgcctacgcaacccacagttttttcgatgaaaatggatttttgtatattcacacccccataataaccaccagcgactgtgagggtgcaggtgagatgttccaagtcactgccttattcagccaaggctgaaaaggtggagaaggagcttaaggagaaccctgcaccatcagaagctgacgttgangctgctaagcttgttgttaaagagaaaggagatgcagttgctcaattgaaagcagcaaaagctagcaagcaagagataactgctgctgtttccgtgcttacaaaagctaaagagatgtgttaagggtggaagangctccaaattgaactggacttcactcaaggatgatggaaatncgtttganaagactctcaacgtcaacttctgacgttcaggcaactnagncnaaattagctgtgnccataagngtnacttggcaaatccggaaagatc

SEQ ID NO: 162 - protein, 139 aa, Triticum aestivum
  UNSURE (99)
AAYPLPKTKITLETLRDFVHLRARTNTIGAVARIRHQLAYATHSFFDENGFLYIHTPIITTSDCEGAGEMFQVTALFSKAEKVEKELKENPAPSEADVXAAKLVVKEKGDAVAQLKAAKASKQEITAAVSVLTKAKEMC

SEQ ID NO: 163 - DNA, 1464 bp, Zea mays
ccacgcgtccgcaccagactgcagaagaaatcaaggaatacagggaaaagaagatggatagtccatggaggggtaggccaattgaagagtctctgaaattatttgaagacatgaggcgtgggttgattgcagagggtgcagcaactctccgtatgaagcaggatatgcaaaatgagaacaaaaatatgtctgacttaatagcatatagaataaaattcaccccccatccacatgctggcgacaagtggtgtgtctatccgagctatgactacgctcattgcatggtggattctcttgaaaacatcacacattcgctgtgcacacttgagtttgacatacgtcgcccttcatactactggctacttgttgccttgggcctttatcaaccatatgtgtgggaatattcgaggctaaacatatcaaatactgtgatgtctaaaagaaagttgaatcgacttgtgacagagaagtgggtagatgggtgggatgatccccgcttgttgacactggctggtctccggcgacggggagtatcatcaactgcaataaattcctttattcgtggaatcgggataacgagaagtgacaatagcttaattcgtgttgatcgtctagaatatcacatcagggaggagcttaacaaaacagcccctcgaaccatggctgttttgcgacctctaaaggtggtaataactaacttggaagaaggaaaagtactagaccttgatggcaaaatgtggcctgatgcttctgatactgatgcttcctcccactataaggttccgttctcaagaactgtctacattgagaaaactgattttcgcctaaaggactcaaaagactactatgggctagcccctggtaaatctgtcatgctaaggtatgcgttccccataaaatgcacagatgttatctctggtgatagtcctgatgatattgttgaaattcgagctgaatatgatcctttgaagacttctaaactgaagggtgttcttcactggattgctgagccagcacctggtgtagagccattgaaggtggaagtaagattattcgagaaattgttcatgtcagagaatcctgctgaattggaagattggcttggcgatcttaacccacactcgaaagaggtgataaaggatgcttatgctgtaccatcacttgccactgcggttctgggtgacaagttccagtttgagcggcttggttacttcgccgtggatactgactccacacctgagaaactcgtgttcaacagaactgttaccctccgtgattcgttcgggaaagctggacccaagtgactgttcagtgtaatttagggagggcgctggttttgatcggttgcagaggcgcacctgaactatacagtttgtgaagaaaatggtcgtctaatacagaacagtttaaaggccttactctttataaaatttagggttttttaaaaaaaaaaaaaaaaaaaag

SEQ ID NO: 164 - protein, 433 aa, Zea mays
TRPHQTAEEIKEYREKKMDSPWRGRPIEESLKLFEDMRRGLIAEGAATLRMKQDMQNENKNMSDLIAYRIKFTPHPHAGDKWCVYPSYDYAHCMVDSLENITHSLCTLEFDIRRPSYYWLLVALGLYQPYVWEYSRLNISNTVMSKRKLNRLVTEKWVDGWDDPRLLTLAGLRRRGVSSTAINSFIRGIGITRSDNSLIRVDRLEYHIREELNKTAPRTMAVLRPLKVVITNLEEGKVLDLDGKMWPDASDTDASSHYKVPFSRTVYIEKTDFRLKDSKDYYGLAPGKSVMLRYAFPIKCTDVISGDSPDDIVEIRAEYDPLKTSKLKGVLHWIAEPAPGVEPLKVEVRLFEKLFMSENPAELEDWLGDLNPHSKEVIKDAYAVPSLATAVLGDKFQFERLGYFAVDTDSTPEKLVFNRTVTLRDSFGKAGPK

SEQ ID NO: 165 - DNA, 1992 bp, Oryza sativa
gcacgagattgaatccatactctatatttcctcagccagaggaaaattttaaggttcatacagaaatattctatagtgatgggaacatatggagagcgcataacagtaaggagattttagagaaacaccttaaggcaaccggtggaaaagtgatgacccgtttcccaccagaacctaatggatatcttcatattggtcatgccaaggctatgtttattgattttggactggcgaaagagagaaatggtcattgttaccttaggtttgatgacacaaatccagaagccgaaaagaaagagtatattgaccacattcaggaaatcgtacactggatgggatgggagccctacaaagttacatatacaagtgattatttccaggctttatatgagcatgcagttgagttaatacgaaaagggctagcctatgtggatcaccagaccgcagaagaaatcaaggaatacagggaaaagaaaatgaatagtccatggagggatagacccattgaagaatcactgaaactatttgaagacatgagacgtgggttgattgctgaaggtgcagcaacactccgaatgaaacaagatatgcagaatgataacaagaatatgtctgatttaatagcatatagaataaaattcactcctcatccacatgctggtgataagtggtgcatctatccaagctatgactatgctcactgcatggtggattctcttgagaacattacacattcgctgtgcacgctcgagtttgacatacgtcgcccgtcatactactggctacttgttgccttgggcctgtaccagccatatgtttgggagtattcgaggctaaacatatcgaatactgtgatgtctaaaagaaagttgaatcgacttgtgacagaaaagtgggtagatgggtgggatgaccctcgtttgttgacactagcaggattgcggcgacgtggagtgtcatcaactgcaattaattcgtttatttgtggaattggaataacaagaagtgacaatagcttaattcgggttgaccgtcttgaatatcatatcagagaagagcttaataaaacagcttcccgtgccatggttgtgttgaatcctctaaaggttgtaataactaacttggaggatgaaaaagtcatagaccttgatggaaaaatgtggcctgatgctcctgcagacgatgcttcatcctactacaaggttcctttctcaagaatcgtttacatcgaaaaaactgattttcgtctaaaggactcgaaagattactacgggctagctcctggtaaatctgccctgctaagatatgcattccccattaaatgtaccgaggttgtttatggtgacaatccagatgacatcattgaaattcgagctgaatatgacccttcaaagactactaaacctaagggtgttctgcactgggttgctcagccagcacctggagttgaaccacttaaggtggaagtaagattatttgataaattattcctctctgagaatcctgctgaactggaggattggctgggtgatcttaacccgaactcaaaagaggtgatcaagggtgcctacgccgtgccatcgcttgcgactgcggttcttggtgacaagttccagttcgagcggctaggctacttcgcagtggacacagactcgacacctgagaacattgtgttcaacaggacggttaccctgcgtgattcgtatgggaaagctgggccaaagtgattgccaacttactgaattttcagtaagtttctttacctgggtgcactgaggttacacagtgggaagaaaactgcagtgtgatacataaccatagtgaaaaaaaagggccctttcttgtaaaatggacttgtttgacttgttatatgtttccatcaaattgtgcatgattgagtatgttctcatgtcaatgaggtttggtgatttgacttctagcatttataaatggtgcaaaccaacattataaacagaaaaaaaaaaaaaaaaa

SEQ ID NO: 166 - protein, 574 aa, Oryza sativa
TRLNPYSIFPQPEENFKVHTEIFYSDGNIWRAHNSKEILEKHLKATGGKVMTRFPPEPNGYLHIGHAKAMFIDFGLAKERNGHCYLRFDDTNPEAEKKEYIDHIQEIVHWMGWEPYKVTYTSDYFQALYEHAVELIRKGLAYVDHQTAEEIKEYREKKMNSPWRDRPIEESLKLFEDMRRGLIAEGAATLRMKQDMQNDNKNMSDLIAYRIKFTPHPHAGDKWCIYPSYDYAHCMVDSLENITHSLCTLEFDIRRPSYYWLLVALGLYQPYVWEYSRLNISNTVMSKRKLNRLVTEKWVDGWDDPRLLTLAGLRRRGVSSTAINSFICGIGITRSDNSLIRVDRLEYHIREELNKTASRAMVVLNPLKVVITNLEDEKVIDLDGKMWPDAPADDASSYYKVPFSRIVYIEKTDFRLKDSKDYYGLAPGKSALLRYAFPIKCTEVVYGDNPDDIIEIRAEYDPSKTTKPKGVLHWVAQPAPGVEPLKVEVRLFDKLFLSENPAELEDWLGDLNPNSKEVIKGAYAVPSLATAVLGDKFQFERLGYFAVDTDSTPENIVFNRTVTLRDSYGKAGPK

SEQ ID NO: 167 - DNA, 1258 bp, Glycine max
ccacgcgtccggctaaatcgtctagttacagagaagtgggttgatgggtgggatgatcctcgtttgatgacactagctggtttgcggcgtagaggcatgaccccaactgcaatcaatgcttttgtccgaggaattggaataactagaagtgatggcactttgatttctgtggaacgccttgaatatcatgttagggaagaattgaacaaaacagcacctcgtgcaatggttgtcctacatccactcaaggttgtcattactaatcttgaagccaactcagcaattgaggttgatgcaaagaaatggcctgatgctcaagctgatgatgcttctgctttctacaagattccattttccaatgttgtatatattgaacattcggacttccggatgcaagattcaaaagattattatggccttgctcctgggaaatctgtgatactcagatatgcatttcctataaagtgcactgaagttattctagctgatgataatgagactattcttgaaattcgagccgagtatgatccttcaaagaagaccaagcctaagggggttctccattgggttgctcaaccttctcctggagttgatccattgaaggtggaagtcagattgtttgagaggctattcctatcagagaatcccgctgaacttgacaactggcttggcgatttgaacccaaattccaaagtgataattcccgatgcatatggtgtgtcttccatacagaatgcaaaagttggggacaatttccaatttgaaagattaggctattttgtggttgaccgggactcgacatcagaaaaacttgtttttaataggactgtcaccttaaagggcagctatagcaaaggtggaaagtagggtccctgaatattgattagaagtaacaaggtgggacaggcaggtctgcgggtaatacttgtttaaatagctacttataatgcatagttttttctttgagggggtggggggttatgtacaaacatatttactggccacttagccatgtacattttcttaagggattactcaatagttccccaattatttaattattttgtttggttggttttgtagtttgaagattttttctgtttagtcccaatgtagtaccaaattgctgttattttcctaccattttgtgtaaaattagttctgaagaatctttccctttacatttaagtgaaaatcaagtctaatttttgttcatgtgaaaaaaaaaaaaaaaaaaaaaaaaaaaaaaaaaaaaaag

SEQ ID NO: 168 - protein, 288 aa, Glycine max
PRVRLNRLVTEKWVDGWDDPRLMTLAGLRRRGMTPTAINAFVRGIGITRSDGTLISVERLEYHVREELNKTAPRAMVVLHPLKVVITNLEANSAIEVDAKKWPDAQADDASAFYKIPFSNVVYIEHSDFRMQDSKDYYGLAPGKSVILRYAFPIKCTEVILADDNETILEIRAEYDPSKKTKPKGVLHWVAQPSPGVDPLKVEVRLFERLFLSENPAELDNWLGDLNPNSKVIIPDAYGVSSIQNAKVGDNFQFERLGYFVVDRDSTSEKLVFNRTVTLKGSYSKGGK

SEQ ID NO: 169 - DNA, 1324 bp, Triticum aestivum
gcacgagaggacatgcagaatgataacaaaaacatgtctgatttaatagcatatagaataaaattcactcctcatccgcatgctggcgacaaatggtgtatctatccaagctatgactatgctcattgcatggtggattcacttgaaaacattacacattctttgtgcacgctcgagttcgacattcgtcgcccgtcatactactggctacttgttgccttgggcttgtaccagccatatgtttgggagtattcgaggctaaacatatcacatactatgatgtccaaaagaaagttgaatcggcttgtgacagagaagtgggtagatgggtgggatgaccctcgtttgttgactttggcaggactgaggcgacggggagtatcagcaactgcgatcaattcatttatccgtggaattgggataacgagaagtgacaatagcttaatccgtgttgaccgtcttgaatatcatatcagagaagaactgaataaaacagcttctcggaccatggttgttttgcatcctctgaaggttgtaataactaatttggaagatggaaaagtcatagaccttgatggaaaaaagtggcctgatgctcctgctgatgaagcttcgtcctactacaaggttcctttctcaaaaaccgtctacattgaaaaaactgattttcgcgtgaaggactccaaagattactatggattggctcctggtaaatctgccctgctgaggtatgcattcccgataaaatgcacagaggttatttatggtgataatccagatgatattgttgaaattcgagccgagtatgacccttcaaagacctctaaacctaagggtgttctgcactgggttgccgagccagcacctggagttgagccgttaaaggttgaaataagattatttgagaaattattcctctcggagaatcccgccgaattgaaagactggctgggtgatattaacccgcactcaaaggaggtagtcaagggcgcctacgctgtaccgtcactcgccaccgcggttctgggcgacaagttccagtttgagcggcttggctactttgctgtggacacggactcgacgcccgagaaccttgtgttgaacaggactgtgaccctgcgcgactcttatgggaaggctggacccaagtgactgcaaatttagtgaagaagctccgtttttcttggtcagagagacgctaatgggagaaaacatagtcatgacatacaacatgaaatggccaagaggccctttttgtttttgttttgcatactgggcttgtgatgactattatagtatgttttgtttcagtcgatcaaaaaaa

SEQ ID NO: 170 - protein, 383 aa, Triticum aestivum
AREDMQNDNKNMSDLIAYRIKFTPHPHAGDKWCIYPSYDYAHCMVDSLENITHSLCTLEFDIRRPSYYWLLVALGLYQPYVWEYSRLNISHTMMSKRKLNRLVTEKWVDGWDDPRLLTLAGLRRRGVSATAINSFIRGIGITRSDNSLIRVDRLEYHIREELNKTASRTMVVLHPLKVVITNLEDGKVIDLDGKKWPDAPADEASSYYKVPFSKTVYIEKTDFRVKDSKDYYGLAPGKSALLRYAFPIKCTEVIYGDNPDDIVEIRAEYDPSKTSKPKGVLHWVAEPAPGVEPLKVEIRLFEKLFLSENPAELKDWLGDINPHSKEVVKGAYAVPSLATAVLGDKFQFERLGYFAVDTDSTPENLVLNRTVTLRDSYGKAGPK

SEQ ID NO: 171 - DNA, 1398 bp, Oryza sativa
gcacgagcttacatcatcccccgcataccacttactccgcttgcatctgccacagaaggaattcaagctgttctggattggttatcccctcaaactccgaacttctcgccttctgggatgccactcatcataagtcagttttatgaaaatttactgagaagcacactttctattgcgagctatgaggcctgttccttcatgggatgcactagctcgatcttaggaacattgacttccttcattgagctctccccttacagaccctgtgggacttatcttttcttgacaagcagtgaacagctggccgttctgacaaattcagatgctgtattgcagttgctattttactgccttcagttggatccccagcaacaattgcgtgatgctgctgagagaagtttaagtgctcattggcaatatgaaccaattaagcaaagcatgatgcaagagatagtttgcgtggattacttgggagtggtttcatcaacacttcctggtaggcagatgagtagcacaatagttggaggccttgaactgagcaaggaagctatgttgagcctttctgcagctggacaatgggagaagcaaagggagacaaaccaggcaaaaatagatggtgcaagctgcaccaaaattagggaagccctcaagtctctgaacgagtacaaaaggacatgtgactccatgaagttggaactagctggactctgggatgagatagttgaaatgctgcgaaggcgtgagctgccagatggtttcgaaagccgacaagattgggtgaatctaggaaccttgtaccgccggttggttgagcccttagacattgcaaactactataggcattccaagaacgaggacactggctcctacctctccaagggaaggccaagacgttacaagtacacccaggagtggcacgagcagtcgcagcgcatctccttcggttctagccttgagtcttgcttctgggcaatggctgaggaactccaggctgagattgctaacggcaaaacattcgaggatgtgagagatagagtagttaagcttgagagtgatgctcacggatggtccatgtctggaagtttagggaaggatatattcctgagccgttcttcttttgtgatttggtggaagacgctcccggagaaccacagatctgcatcatgcatcgcgaaacttgtgccctggtaaggaagctttacattatatggatagaattatttgccttcacaatgatgaggcaggcattcgtggcatgttgcaactactgccactgtaactgtataccatatgcatgaactttgtaaatatgctacttcgcgtttttgtatcctacacaatacaatcagttcaagttgaaccatttgtttcttgcatcaaaaaaaaaaaaaaaaaaaa

SEQ ID NO: 172 - protein, 396 aa, Oryza sativa
ARAYIIPRIPLTPLASATEGIQAVLDWLSPQTPNFSPSGMPLIISQFYENLLRSTLSIASYEACSFMGCTSSILGTLTSFIELSPYRPCGTYLFLTSSEQLAVLTNSDAVLQLLFYCLQLDPQQQLRDAAERSLSAHWQYEPIKQSMMQEIVCVDYLGVVSSTLPGRQMSSTIVGGLELSKEAMLSLSAAGQWEKQRETNQAKIDGASCTKIREALKSLNEYKRTCDSMKLELAGLWDEIVEMLRRRELPDGFESRQDWVNLGTLYRRLVEPLDIANYYRHSKNEDTGSYLSKGRPRRYKYTQEWHEQSQRISFGSSLESCFWAMAEELQAEIANGKTFEDVRDRVVKLESDAHGWSMSGSLGKDIFLSRSSFVIWWKTLPENHRSASCIAKLVPW

SEQ ID NO: 173 - DNA, 2248 bp, Glycine max
cccccgggctgcaggaattcggcacgaggtgaaactacaaggtcatggtcaattcagtagtaattcaatatgtcctagttagttccccctttatataataccaagatgcagaagttctgaaaattaaccaaactcaaaaatttccaaaactttatagaattagagggacaacatggctggagggttgcttggagataaccttggattgaaggaagatgtgatcaagagggtgtgtggtttggcctccaaagctcacaatcacaagtcaacggacaagctctacttttacgataaggttcggacttcttcggggacataccatgttttcagcttctcgggatcttgggatcccgctgaatggttttttagcaaaccctttggcggatccaagatagatcctacccaatttccttcactcagaagtattggtaacgatgaacctgctttggtgaacgaaggcttcgcaaagagattcgatcgcgtattgaaaactagctttaaagccgaggtgaataaggctattggagatgggaagcaagtagtgtttacggggcactcctctggtgctgcaatagccattcttgctaccttttgggcattggaagagtatcttaaccctacaaaaatccaaaaacccacgccacccttttgtgtcacttttgggtctcccttaattggcaatcatatattctctcacgcttcaaggagagaaaattggtctcgctatttcatacactttgttttgagatatgacatagtgccaaggattttgctttctcgcttggcttctattaagcaaacttttggttctgttctccaattcttgaatcccaattccaaaacttccacccaggatccaacaagggctagtttaatttctgaattttacaaaactgtgatgacaaacgcagcgagtgttacaagccatgctgcgtgtattctcatgggaagcacaagtttgttacttgggacagtggcgaattttgttgagttgagcccttataggccctttggaacatttattttctgcaacggaaatggacaattgattgtggtgaaaaactcagatgctgttttgcaactcttgttccacactgctcagatgagcgatttggcagaacttccagaagttgccaatgttagcatattgcaacaccaggcctatgaggctgaattggatgatagcttgggaatgcagaatgtagtgtacttggagcaactagagcaacttccgttgtctgctgatggttctaatagcgatgttgcaacaatcagtgcagccttggatggccttggactgagcacaagagcaaggctgtgtctacgagcagctggtgagttggaaaagcagaagctaaaaaatgaggagaaaatcaagaaggagattcaggagaaagccgtgccaagcatgacgaagcttcaaaattacaaaacaacatgtgagatgcacaaggggaagggctattacgatgccttcaaggtgcaaaatgaggaaaacgacttccaagcaaatgtgaagaggcttgtgttagcaggggtatgggatgaagtgattgagatgctaaagaggtatgaactcccagacgagtttgaagggaattcaaaatggattgaacatggaactgaatttcgtcgccttgtggagcctctagacattgcaaactatcaccgccacttgaagaatgaggacacaggaccttacatgataagggccaggccaaaacggtataggtacactcaaagatggttggagcatgctaaaagggtgccaaaacctgctcctatcactgaatcaaccttttgggctgaagtagaagagctttatagctggattaacagcaagaggcatcttgatgatgaagtaaagcaaagggttgtgcagcttcagaaagaccttaaaaagtggactgatgatgagaaggtactaaccaaggatacgttcttgaaggatcctaactttattaggtggtgggatattctacctcaggaactcagggtcacttccttctagtcttgttactgctgtggaaggatagtgaattggtgtttttttcccctctaaattaatggagaagctatagaaatttcggcatgcaggtgcagtgttggctacttcggtgttgtaactttgtattcccaatgtttatgattcccaagttttgtatgacttgtgagtgagaaccccaaatcattcagactgttgaactaaataaatttgtttaatctagggtctgtattagtgggttag

SEQ ID NO: 174 - protein, 612 aa, Glycine max
MAGGLLGDNLGLKEDVIKRVCGLASKAHNHKSTDKLYFYDKVRTSSGTYHVFSFSGSWDPAEWFFSKPFGGSKIDPTQFPSLRSIGNDEPALVNEGFAKRFDRVLKTSFKAEVNKAIGDGKQVVFTGHSSGAAIAILATFWALEEYLNPTKIQKPTPPFCVTFGSPLIGNHIFSHASRRENWSRYFIHFVLRYDIVPRILLSRLASIKQTFGSVLQFLNPNSKTSTQDPTRASLISEFYKTVMTNAASVTSHAACILMGSTSLLLGTVANFVELSPYRPFGTFIFCNGNGQLIVVKNSDAVLQLLFHTAQMSDLAELPEVANVSILQHQAYEAELDDSLGMQNVVYLEQLEQLPLSADGSNSDVATISAALDGLGLSTRARLCLRAAGELEKQKLKNEEKIKKEIQEKAVPSMTKLQNYKTTCEMHKGKGYYDAFKVQNEENDFQANVKRLVLAGVWDEVIEMLKRYELPDEFEGNSKWIEHGTEFRRLVEPLDIANYHRHLKNEDTGPYMIRARPKRYRYTQRWLEHAKRVPKPAPITESTFWAEVEELYSWINSKRHLDDEVKQRVVQLQKDLKKWTDDEKVLTKDTFLKDPNFIRWWDILPQELRVTSF

SEQ ID NO: 175 - DNA, 1532 bp, Triticum aestivum
  unsure (286)
gcacgaggtgacctttggggcacctcttgttggcgataatgttttcaaccatgctgttagaagggagggctggtcgcagtgtattttgcatttcatcatgccactggacatcatcccgcgaatactgctggcccccctcgcatcctctagagaacaaattcagtccgttctggattggttatctcctcacagtccaaacttctcacctgttgggaattcgcttgttattccagagttttatgaaactttgttgagaagcaccttatctattgccagctacgaggcntgttctttcatgggatgcactagctcaatcctaggaacgctgacttactttattgagctatccccgtacagaccctgtgggacttaccttttcttgacaagtaccgatcaactgattgttctcacaaactcagatgctgtattacagttgctattttactgccttcaattggatccccaacaacaattgcttgatgctgccgccagaagtttaagtgctcactgggaatatgaatcgattaagcaaagtgtgatgcaagagataggttgtgtggattacctaagagcaatttcatcatcccttcttggcacacagatgaatgggacagcaattggtggccttgaactgagtaaggaagctatgctgagccttgctgctgctgcacaatgggagaaacaaagagagataaaccaagcaaagatagacgcaaactgcagtaaaattcaggaagccctcaagtccctgaacgagtacaaaagaacatgcgagctgcatgaagtgagctactatgattccttcaagcttcagcgggaagtgcatgacttcaattcaaatgtacggaggttggaactagccggcttttgggacgagataattgagatgttgcgaaggcgtgagcttccagacgcgttcgaagggcgagaagagtgggtgaacctggggacctcgtaccgccggctggttgagcccctggacattgcaaactactacaggcattccaagaatgaggacaccggctcctacctctccaagggcaggccgagacgttacaagtacacccagaaatggcgcgagcagtcgcatcgcatccccttgggttccagcctcgagtcgtgcttctgggcaatgtccgaggagctgcaggctgagatgatcaacggcaaatcattcgaggatctgaaagacagggtgggtaaactagagagcgatgcacttggatggttcacctcgggaaatcttggcagggatgtgtttctgagcagctcatcctttgtgatatggtggaagacgctcccggagcagcacaggtccgcgtcttgcatcgcgagacttgtgccttcgtaagagaatttctccgtcgcttgatgaggtagccatttccgatctaatcggaactgctggcactggatattgtaagcctgaactctcatgtaaagtcatttgtgtgtagtattctccaggatgaacagactatttaaaatgctaaacagttgttgtccaataaaaaaaaaaaaaaaaaaaaaaa

SEQ ID NO: 176 - protein, 449 aa, Triticum aestivum
HEVTFGAPLVGDNVFNHAVRREGWSQCILHFIMPLDIIPRILLAPLASSREQIQSVLDWLSPHSPNFSPVGNSLVIPEFYETLLRSTLSIASYEACSFMGCTSSILGTLTYFIELSPYRPCGTYLFLTSTDQLIVLTNSDAVLQLLFYCLQLDPQQQLLDAAARSLSAHWEYESIKQSVMQEIGCVDYLRAISSSLLGTQMNGTAIGGLELSKEAMLSLAAAAQWEKQREINQAKIDANCSKIQEALKSLNEYKRTCELHEVSYYDSFKLQREVHDFNSNVRRLELAGFWDEIIEMLRRRELPDAFEGREEWVNLGTSYRRLVEPLDIANYYRHSKNEDTGSYLSKGRPRRYKYTQKWREQSHRIPLGSSLESCFWAMSEELQAEMINGKSFEDLKDRVGKLESDALGWFTSGNLGRDVFLSSSSFVIWWKTLPEQHRSASCIARLVPS

SEQ ID NO: 177 - DNA, 757 bp, Zea mays
  unsure (542)
  unsure (603)
  unsure (678)
  unsure (725)
  unsure (744)
ggatctaaccaaaccccgcgccctcctcaccgtccggcgagctacggactcagcagatcaccgtcgctcgagttgtacctgaaggcgcgcccgtggaaccggccgcgagataagggcggcgggaaggcgggcgacgatgccggtggcagcgtcggccatctacttcctcaaccttcgcggggacgtcctcatcaaccgcctctaccgtgatgatgttgggggaaatatggttgatgcgttcagaatgcatatcatgcaaacaaaagaacttggcacatgccctgttcgtcaaataggaggctgctccttcctttatatgaggatcagtaatgtttacattgtgatcgtagttagcagcaatgctaatgttgcatgtgctttcaaatttgttgtcgaggcggtggctctcttcaagtcctacttcggtggagcttttgatgaagacgctatcaggaataactttgttttgatatatgaacttcttgatgagatcatggattttggttatcctcaaaacctgtcacctgaaattttgaagttgnatataactcaagaaggtgttcggtcaccattttcctcaaaagcccttaaaagaacctgttncaaatgcgactcttcaagtcactggcgcttgttgggttggagaagaaaaggacttgtgtaccaagaaagaatganggtttcttggacatagttgaaaagtgggaacccttcttatggcttcnaaaagggagtggtcttaanaatgtgatgtgac

SEQ ID NO: 178 - protein, 185 aa, Zea mays
  UNSURE (136)
  UNSURE (156)
  UNSURE (181)
MPVAASAIYFLNLRGDVLINRLYRDDVGGNMVDAFRMHIMQTKELGTCPVRQIGGCSFLYMRISNVYIVIVVSSNANVACAFKFVVEAVALFKSYFGGAFDEDAIRNNFVLIYELLDEIMDFGYPQNLSPEILKLXITQEGVRSPFSSKALKRTCXKCDSSSHWRLLGWRRKGLVYQERMXVSWT

SEQ ID NO: 179 - DNA, 831 bp, Oryza sativa
gcacgaggtttaaactatcgaatcactgagggtgtaaatctcccattccgggttctaccaacgattaaggaattgggccgaacacgcatggagataaatgttaaagttaagagtgtttttggtgctaagatgtttgcacttggtgttgtcgtcaaagtccctgttccaaaacaaacagcaaagactagtttccaaaccacatctgggaaagccaaatataatgcttcaattgattccctagtgtggaagatcaggaagtttcctggacaaactgaggcaaccatgagtgccgaagttgagctgatttctacaatgggggaaaagaaatcatggaacaggccgccaattcagatggaattccaggttcctatgtttactgcttctgggttacgagttcggttccttaaggtgtgggagaagagcggttacaacaccgttgagtgggttcgctacatcacaagggctggatcatatgagataaggtgttagcgatgaagaaaaaagtcccaggtatttggcacggttatttcaaggattcgcggagctttctttctagtacggcgtcatgaatgatcataaagcatgtatattacatatgacaccactttttctttaagctattactttggcaaagggtagagcagagcagacaaattttcctgggttcttgtaatcgttttgacacgggatatctagtatcccctgagtcttgtttgtgtggtagggcatattttcatttgtatatgagaagatggcggtagcgtttgttcttgttgatcaatatacatatactcattggcagttaaaaaaaaaaaaaaaaaaaaaaaaaaa

SEQ ID NO: 180 - protein, 162 aa, Oryza sativa
ARGLNYRITEGVNLPFRVLPTIKELGRTRMEINVKVKSVFGAKMFALGVVVKVPVPKQTAKTSFQTTSGKAKYNASIDSLVWKIRKFPGQTEATMSAEVELISTMGEKKSWNRPPIQMEFQVPMFTASGLRVRFLKVWEKSGYNTVEWVRYITRAGSYEIRC

SEQ ID NO: 181 - DNA, 1776 bp, Glycine max
gcacgagctgaattgtcaaattcacccgctaccttttcccgtgtacgaacgaatccaatccaccaaatcggcggcgacgcctccgctctgtctcgaaaccacagatctgcggcggaaccgccgtctccgactatctccgattgatcttccggtaaagtggaccggagatgccggttgccgcttccgccatttacttcctcaacctccgcggcgacgttctcatcaaccgcctctatcgcgacgatgtcgggggaaatatggtggatgcttttaggacgcatattatgcaaacgaaggagcttggtacttgcccagtgaggcagattggtggatgctctttcttttacatgaggatcagtaatgtctacattgtcattgttgtcagcaacaacgccaatgtcgcttgcgctttcaagtttgttgttgaggctgttgcattgttccgatcatactttggtggagcttttgacgaggatgcaattcgcaataattttgtactcatttatgagctcctagatgaaattatggactttggttacccacaaaatctttcaccagagatcttaaagctttatatcactcaggaaggagtgcgttccccattttcatcaaagcccacagatagacctgttccaaatgcaactttacaagttactggtgctgttggttggcggagagaagggcttgtgtacaaaaagaatgaggtcttcctggatattgtggaaagtgtaaatcttctaatgtcttcaaaaggtagtgttctgcgttgtgatgtcactgggaagattctcatgaagtgctttctttctggaatgcctgatttaaagttgggtttgaatgacaagattggtcttgagaaagaagcacaacttaaatcccgtcctactaaaagtggtaaaagtatcgagcttgatgatgtcactttccatcaatgtgtaaatttgacaaggttcaactcagagaagacagttagttttgtaccacctgatggtgaatttgaactaatgaagtatcgtataactgagggagttaatcttccgttcaaagtattgccaaccatcaaggaacttggtcgatcacggatagaagtgaacgttaaggtaaagagtgtttttggtgcaaaaatgtttgcacttggggttgtagtcaagattcctgttccaaaacaaacagcaaaaacaaatttcacagttacatctggccgggcaaaatacaatgcttctattgattgtttggtttggaagattagaaaattccctgggcagactgagtcaaccttaagtgcagaagttgaacttatttccacaataacagaaaagaaatcttggactagaccaccaattcagatggagtttcaggttcccatgttcacagcatctggtttacgtgttcgtttcctcaaggtgtgggagaagagtgggtacaataccgttgagtgggttcgttacattacaaaagctggatcctacgagattaggtgctagacagagtttgtattactggatggagaatgaagcaaaatcccgaataagccgcagttcaggctagtcatcctagttttcgtattgtattaaacatgtaatgaaaggttcaggagttactcaatgtgctgtcagtatgattttttgtgtgaaaatctgtttttaatatttttgttccaaaaggccagcttgttattggaacctgaagttgagtgttctcttgtaaactttgtattttgttcttatatctctttttatataaaacatcatttattgtaaaaaaaaaaaaaaaaaa

SEQ ID NO: 182 - protein, 438 aa, Glycine max
MPVAASAIYFLNLRGDVLINRLYRDDVGGNMVDAFRTHIMQTKELGTCPVRQIGGCSFFYMRISNVYIVIVVSNNANVACAFKFVVEAVALFRSYFGGAFDEDAIRNNFVLIYELLDEIMDFGYPQNLSPEILKLYITQEGVRSPFSSKPTDRPVPNATLQVTGAVGWRREGLVYKKNEVFLDIVESVNLLMSSKGSVLRCDVTGKILMKCFLSGMPDLKLGLNDKIGLEKEAQLKSRPTKSGKSIELDDVTFHQCVNLTRFNSEKTVSFVPPDGEFELMKYRITEGVNLPFKVLPTIKELGRSRIEVNVKVKSVFGAKMFALGVVVKIPVPKQTAKTNFTVTSGRAKYNASIDCLVWKIRKFPGQTESTLSAEVELISTITEKKSWTRPPIQMEFQVPMFTASGLRVRFLKVWEKSGYNTVEWVRYITKAGSYEIRC

SEQ ID NO: 183 - DNA, 554 bp, Triticum aestivum
gcacgagatttcctggacagaccgaggcaacgatgagtgcagaagttgaactgatctctacaatgggggaaaagaagttagcgaacaggccaccgattcagatggagttccaggttccgatgttcactgcttctggcttacgtgttcggttcctcaaggtgtgggagaagagtggctacaacaccgttgagtgggttcgctacatcacaagggctggatcatatgaaatcaggtgttagtgaccaagaaaaatggcgtgggctcattatttcgtggatttgcagagctctttttgtactcatcatgattgatcagatggcacgtaaattatgacccaattcgttgcaagtttgaacttcagcagagggcagatcagagcaggcagttttcctatgttccttattttcatgttgacatgggatatgtattctcccccgtgtcctgtttgtcatagggcatatttttattgtatatttggcagtagcattcgttctcgttgatcaatacacccccttgccagttcgtgctgttttctcaaaaaaaaaaaaaaaaaa

SEQ ID NO: 184 - protein, 78 aa, Triticum aestivum
TRFPGQTEATMSAEVELISTMGEKKLANRPPIQMEFQVPMFTASGLRVRFLKVWEKSGYNTVEWVRYITRAGSYEIRC

SEQ ID NO: 185 - DNA, 3830 bp, Zea mays
ccacgcgtccggccgcggacgaatcaaaaaagcttcctcctcccggcgtcgcctccgcgcgtggaggcgacgatccattccgccccccgtaggattttgctcctcccgatccggacgtcagatctcgagtggcggcacgcggatctgggggtttctctagaggcggcggcggcggccggaacaggggcagatggcgctctcggggatgcgggggctctcggtcttcatcagcgacatacgcaactgccacaacaaggagcaggagcgcctccgcgtcgacaaggagctcggcaacatccgcacgcggttcaaaaacgagaagggcttatcaccatacgagaagaaaaaatatgtatggaaaatgctttacatttatatgctgggttatgatgttgattttgggcatatggaaactgtctcgttgatatctgcaccaaagtatcctgagaagcaggttgggtatattgtgacatcttgcttactgaatgagaataatgatttcttaagaatggtaataaatacagtaaggaatgacataatcggacgaaatgagaccttccagtgcttagcattgacaatggtaggtaacattggtggtaaagaattttctgagtcactagccccagatgtccagaaacttcttatttcaagcagttgccgtcctgttgtcagaaagaaggctgctctatgccttctgcggctttataggaagaatcctgatgttgtgaatattgatggctgggctgaccgaatggcacaacttctagatgagcgtgatttaggtgtgctgacatctgtcacgagtctttttgtttcactagtatccaacaatgttgaagcatattggaattgtcttcctaaatgtgtgagaatattggagaggttagcaagaaatcaagatattccacaagagtacacttactatggaattccatctccttggcttcaggttaagacaatgagagctcttcagtacttccctaccattgaagatcccaacgcaagacgagctttgtttgaggttttacagcgcattttgatgggtactgatgttgttaaaaacgttaacaagaacaatgcctcacatgctgttctctttgaagctcttgctctggtcatgcatctggatgctgaaaaggagatgatgtcacagtgtgtggctcttcttgggaagtttattgcagtccgggagccaaacattcggtaccttggtctggaaaacatgactaggatgctgttagtaacagatgtgcaggatatcattaaaaggcaccaggctcagatcatcacatctttgaaggatccagatatcagcattaggagaagggctcttgatttattatatggcatgtgtgatgttacaaacgcgaaggaaattgtggaggagttgttgcagtaccttgatacagcagaatttgcaatgcgtgaagagttgtctctgaaggcagctattcttgcagaaaaatttgctccgcaactattgtggtatgttgatgttattcttcaacttatagacaaagctggagattttgtaagtgatgatatatggtatcgagtggtacaatttgttacgaacaacgaagacctacagtcatacgctgcaactaaagcgagagaatatcttgacaagcctgctttacatgagacgatggtcaaggtgagtgcctaccttcttggggagtatggacatcttttggcccggagacctggctgtagccctaaggaattgtttgctattataaatgataaacttcctacagtatcggcaagtactgtagctattcttctctcaacctatgccaagatattgatgcacactcaacctcctgatgttggactgcaacaacaaatcctcacaatatttaaaaagtacgagagctatattgatgttgaaatacaacagagagcagttgaatattttgaactaagcaggaaaggtcctgctttggcagatgtgttggctgaaatgccaaaattccctgaacgtgagtctgcactgttgaagaaggctgaagacgctgaaattgacacagcagagcaaagtgccataaagctacgcagtcagcaacaaacatctagtgctcttgttgtagctgatcacccccctgcaaatggatttgcaccaccagttaataatctcactcttgtgaagatgccaagtcaaactgtttctgacactcaggagagtggagtaagttatgaagaagctcctaagcctcctgttgaagctcccaaagaaaatgggactcctgtcgaagtgcagaacagggacacaaacattactgaaatcaacaatgagattaaagctgaacctccctctacatcccattctacctctcccggagacctcctcctggcagatcttttgggtcctcttgcgatagaaggccctcctgctgtagagcaaaatcctgcccaaggattgaatgctaatcaaagtccagctggtgacttggcacttgctacccttgatgatcagtccaactcagttcagccaattgtcaacgtcgaggagaagtttcatattttgtgcacaaaagatagtggggtgctgtacgaggatccttacatccagattggcttgaaagcggagtggcgtgcccatcatggccgtcttgttcttttcctgggtaacaaaaatacttcagcccttacgtcagtgcgggcgctgattttgcctcctagtcatttgaaaatggaactttcgtcagttcctgatactattcctccaagagcgcaggtgcaagttccccttgaggttgcgaatctccttgcaagtagagatgttgcggttctagatttctcgtatgcttttgggacttcactggtggatgccaaacttcgactccctgttgtattgaataaatttttgcagactataactcttacccctgaagaatttttctcccagtggaaagcgcttactgttcattcgctgaaggttcaagaagtggttaaaggtgtgaaacccatgcctctttctgagatggctaatcttttcatgagccttcacttggcggtcgctcctggacttgataacaacccgaacaatctggttgcatgcacgacattcttttctgaagcaacacgtgccatgctttgtctgatacgagttgaaacagatccacaagacaggacccaactgcggctaacagttgcctcgggcgaccaatatttgacattcgagttgaaagagttcatcaaagaacatctggttgatatcccaaggactcaggtcgccccacctcgcgcagcagtccagccacagttgcctactacagcacctgccacatacaatgaccctggtaccatgcttgctggattgctatgagaagtaacccgaaggaatagtgttttgatgtacatgacagttatttaatacgaggatctaacatctccacaagcatcttgctgccaagacgtacacgtggagcaaccaagaaagagctggcggagtgtagtctccatgaggattctaaaccgagaaagagcttagattctttttgtgcggtttgaaggccccctccctctccatgtagcagttgccgtattgtgtaagcgtaacttgttgtttctcattattatcatccccttttggtccatttgtataacctgtatcgcccatgtctggtggtggacaatcggtagtttttgtgtgttaatataaggtgctaggttttcccaccatgccgattgtaaagttgccctggtgcgcattgcactgcgatttgtacggaatagggaaggaaaaatttataggttttgctttgtgcatgttcaaaaaaaaaaaaaaaaaaaaaaaaaaaaaaaaaaaaaaaaaaaaaaaaaag

SEQ ID NO: 186 - protein, 1046 aa, Zea mays
MALSGMRGLSVFISDIRNCHNKEQERLRVDKELGNIRTRFKNEKGLSPYEKKKYVWKMLYIYMLGYDVDFGHMETVSLISAPKYPEKQVGYIVTSCLLNENNDFLRMVINTVRNDIIGRNETFQCLALTMVGNIGGKEFSESLAPDVQKLLISSSCRPVVRKKAALCLLRLYRKNPDVVNIDGWADRMAQLLDERDLGVLTSVTSLFVSLVSNNVEAYWNCLPKCVRILERLARNQDIPQEYTYYGIPSPWLQVKTMRALQYFPTIEDPNARRALFEVLQRILMGTDVVKNVNKNNASHAVLFEALALVMHLDAEKEMMSQCVALLGKFIAVREPNIRYLGLENMTRMLLVTDVQDIIKRHQAQIITSLKDPDISIRRRALDLLYGMCDVTNAKEIVEELLQYLDTAEFAMREELSLKAAILAEKFAPQLLWYVDVILQLIDKAGDFVSDDIWYRVVQFVTNNEDLQSYAATKAREYLDKPALHETMVKVSAYLLGEYGHLLARRPGCSPKELFAIINDKLPTVSASTVAILLSTYAKILMHTQPPDVGLQQQILTIFKKYESYIDVEIQQRAVEYFELSRKGPALADVLAEMPKFPERESALLKKAEDAEIDTAEQSAIKLRSQQQTSSALVVADHPPANGFAPPVNNLTLVKMPSQTVSDTQESGVSYEEAPKPPVEAPKENGTPVEVQNRDTNITEINNEIKAEPPSTSHSTSPGDLLLADLLGPLAIEGPPAVEQNPAQGLNANQSPAGDLALATLDDQSNSVQPIVNVEEKFHILCTKDSGVLYEDPYIQIGLKAEWRAHHGRLVLFLGNKNTSALTSVRALILPPSHLKMELSSVPDTIPPRAQVQVPLEVANLLASRDVAVLDFSYAFGTSLVDAKLRLPVVLNKFLQTITLTPEEFFSQWKALTVHSLKVQEVVKGVKPMPLSEMANLFMSLHLAVAPGLDNNPNNLVACTTFFSEATRAMLCLIRVETDPQDRTQLRLTVASGDQYLTFELKEFIKEHLVDIPRTQVAPPRAAVQPQLPTTAPATYNDPGTMLAGLL

SEQ ID NO: 187 - DNA, 1527 bp, Glycine max
ggatgaggcagatcaaagattatcccaggaaaatgggactttgagtatagtagattctcaacctccctctgcagatctccttggcgatctcttgggtccactggctattgaaggccctcccagtagcagtgtccacctgcagccaagctcaaattcaggagtggaaggaactgtagttgaagccacagccatagtacctgctggagaacaggccaattctgtacagccaattggaaatattgctgaaagatttcatgctttgtgcgtgaaggatagtggtgttctatatgaggatccttatattcagattggcattaaagcagaatggagagctcatcaagggcatcttgttcttttcttgggaaacaagaatacttctcctcttgtctctgttcaagctttaatattgcctcccacgcatttgaagatggagctctctctagtaccagaaactatacctccccgggcacaagtgcaatgcccacttgaggtcattaatctccacccaagcagggatgttgccgttcttgacttctcctacaagtttggtaacgatatggtcaatgtcaagcttcgcctacctgctgtcctaaataaatttcttcagcctataactatatctgctgaagagtttttccctcaatggagatcacttcctggaccacctttgaaacttcaagaagtggtcagaggtgttagaccccttccactgctcgaaatggcaaacttattcaatagttaccatttgacagtttgcccagggcttgatcccaatcctaacaatcttgttgtgagtacaacattttattcagaaagtacaagggcaatgctttgtttagtaagaattgaaacagatccagcagacagaacccagctgcgaatgacggttgcttcaggggacccaacactaacatttgagatgaaggagttcatcaaggaccaattagtcagcattcccgccatagcaacccgtgtaccaacacaaccagctccgacatctccaccactggcccaaccaggcagtgctcctgcagcattgacggatcctggggcaatgctggctgctcttttatgacaatatagaggtgttcattaattattacacatttgtaaacctgatctacacagcctaacccatgtctccctcaaagctagtgaggtgaatagacaaaatcatgatgcaagggctggcggaattaagagtgagcttgtttaccagctgagtgtttgagcagggatttctgggttggtgggcatttttgagattctttctgcacaaagtgattgtcctttaagcaagattttatatcagttgggcgagggttttcagttttctcatatttttccccttcccagtttcactggattgtttgtggtgcacctgatagcatgtaagatattgcctttgatagaaacaattgtaagtttgaaattgatactttgacgttacatacaataatagcttgtggagtaaggacatacgcatttttgctatcaaatgctatctagggttcaaacttcaaaaaa

SEQ ID NO: 188 - protein, 357 aa, Glycine max
DEADQRLSQENGTLSIVDSQPPSADLLGDLLGPLAIEGPPSSSVHLQPSSNSGVEGTVVEATAIVPAGEQANSVQPIGNIAERFHALCVKDSGVLYEDPYIQIGIKAEWRAHQGHLVLFLGNKNTSPLVSVQALILPPTHLKMELSLVPETIPPRAQVQCPLEVINLHPSRDVAVLDFSYKFGNDMVNVKLRLPAVLNKFLQPITISAEEFFPQWRSLPGPPLKLQEVVRGVRPLPLLEMANLFNSYHLTVCPGLDPNPNNLVVSTTFYSESTRAMLCLVRIETDPADRTQLRMTVASGDPTLTFEMKEFIKDQLVSIPAIATRVPTQPAPTSPPLAQPGSAPAALTDPGAMLAALL

SEQ ID NO: 189 - DNA, 3202 bp, Zea mays
ccacgcgtccgatcgcaagcgtccacaccgtgaccaccggcgccgctgcggcgtccggagcaggcggcgagcgtcgtccacagggtaggctcggctcgctgaggcggacgagatgagcgggcacgactccaagtacttctctaccaccaagaagggggagatccccgagctcaaggaggagctcaactcccagtataaggacaagagaaaagatgctgtcaagaaagtgattgctgctatgactgtaggaaaggatgtctcatcattgttcactgatgttgtgaactgcatgcagactgagaacttggagctcaagaaactagtatatttgtatctcatcaactatgctaaaagtcaacctgatcttgccattcttgctgtgaacacatttgttaaggattcacaagacccaaacccattgattcgtgctttggctgttaggacaatgggttgtatccgcgtggacaaaatcacagagtatctctgtgatccacttcaaagatgcctcaaggatgacgatccgtatgtacggaagactgcagctatttgcgttgctaaactttatgatataaacgctgagctagtagaggacagaggatttctggaggcccttaaggacttaatatctgacaataatcctatggttgttgcaaatgctgttgctgctctggcagagattcaagatagtagtgttcagccaatctttgaaatcaccagccatacactgtcaaagcttctgacagctttgaatgaatgcacagagtggggccaagttttcattttggattctttgtcaagatataaagcagcagatgccagggaagctgaaaacatagtggaacgagttacaccccgtctccaacatgcaaattgtgcggttgttctttctgctgtcaagataatccttctacaaatggagctcattacgagcacggatgtagtcaggaatctctgcaagaaaatggctccccctcttgttactcttttgtcagcagagcctgaaattcagtatgtagccttgaggaacattaatctgatagttcaaaagaggcctacaatactcgctcatgagattaaggttttcttttgcaagtacaatgaccctatatatgttaagatggaaaagctggagattatgataaagcttgcctcagatcgaaatatagatcaggtgacttctctctgttcaagcgttgacagagcttcttgtaggtgctcttggaattcaaggagtatgccacagaggttgatgttgattttgtgcggaaagctgttcgtgcgattgggagatgtgcaattaaattggagagagctgctgaaaggtgcatcacgcgttttgctcgagctgattaagataaaagttaattatgttgttcaggaagctataattgttatcaaagacatcttcagacgctaccctaatacgtatgagtctatcattgctacactttgtgaaagtctggacactttagatgaaccagaggctaaggcatccatgatttggataattggagaatatgctgaaagaattgacaatgcagatgaacttcttgagagcttcttggaaacattccctgaagaaccagcattagttcaactgcagctgctaaccgctactgttaaattgtttcttaagaagccaacagaggggccacaacagatgattcaggctgttctcaataatgcaacagttgaaacagataatcctgatttgagggacagagcttacatatattggcgacttctgtctactgatcctgaggctgcgaaagatgttgttttggcggagaaacctgtgatcagtgatgactccaaccagcttgactcatcacttcttgatgagctgctagcaaacatttctaccctttcatcagtttatcataagcccccagaatcatttgtcagccgtgttaaggctgctcctagggccgatgatgaagagtttgctgatacagctgaaacagggtactcggagtccccatcccagggtgttgatggggcatcaccttcatctagtgctggtacttcttctaatgtacccgtgaagcagctggcggttgcatccccacctgcaatgccagaccttttaggtgatttgatgggtatagataatgctattgttcctgttgatgaacctgcagcaccttccggccctccactacctgtcttactgccttcgactacaggtcaaggactgcaaattagtgcacaactaacacggcgtgatggccagatatactatgacatatcttttgagaatggcacccaaggtgtcctagatggatttatgattcagtttaacaagaacacatttggtcttgctgctggtgaagcacttcaggttactccactgcaaccaggccaatcaacaaggacacttttacaaatgaccccgttccagaatatcagccctggtgcaccaaactcgctactacaggttgctgtgaaaaataatcagcagccagtgtggtacttcaatgacaaaattccgctgcatgttttctttggtgaagatggaaaaatggaacgagctggttttcttgaggcctggaaatctttgcctgatgacaatgaatttacaaaagaattcccgggctctgtcatcagcagcatagatgctactgttgagcgcctcgtagcatcaaatgtgttcttcatagccaagcggaaaaatgcgaacatggatgttctgtatctctccgcgaagatgccccgtggaatccccttccttatagaggttacagccgtggttggtgttcctggtgtgaagtgtgcagtcaaaacaccaaatagggagatggttcctctcttctttgaagctatggaggctctcaccaagtgacgacaaacttaatggatctggatcggtggttctacaaaaaaaataccagtcgatgagctgctataggtgtttggacgtggcattttatttttcacggaagctggtgtgaattgtagtttttttggtattagattacagtatttaaactgctagtttcctggttccaaagttttttcaccagaaatactggaggtgttcatgccttgcatgatttgtacatcttaccatgttgtatgaagcgatgaaattgtaggtgacgaaaaagtagaataagcatagattaactggtacgattgtggattgttattaatttccctgcaacccaatcactattttaatggtgatgtatctatatttttc

SEQ ID NO: 190 - protein, 909 aa, Zea mays
MSGHDSKYFSTTKKGEIPELKEELNSQYKDKRKDAVKKVIAAMTVGKDVSSLFTDVVNCMQTENLELKKLVYLYLINYAKSQPDLAILAVNTFVKDSQDPNPLIRALAVRTMGCIRVDKITEYLCDPLQRCLKDDDPYVRKTAAICVAKLYDINAELVEDRGFLEALKDLISDNNPMVVANAVAALAEIQDSSVQPIFEITSHTLSKLLTALNECTEWGQVFILDSLSRYKAADAREAENIVERVTPRLQHANCAVVLSAVKIILLQMELITSTDVVRNLCKKMAPPLVTLLSAEPEIQYVALRNINLIVQKRPTILAHEIKVFFCKYNDPIYVKMEKLEIMIKLASDRNIDQVTSLCSSVDRASCRCSWNSRSMPQRLMLILCGKLFVRLGDVQLNWRELLKGASRVLLELIKIKVNYVVQEAIIVIKDIFRRYPNTYESIIATLCESLDTLDEPEAKASMIWIIGEYAERIDNADELLESFLETFPEEPALVQLQLLTATVKLFLKKPTEGPQQMIQAVLNNATVETDNPDLRDRAYIYWRLLSTDPEAAKDVVLAEKPVISDDSNQLDSSLLDELLANISTLSSVYHKPPESFVSRVKAAPRADDEEFADTAETGYSESPSQGVDGASPSSSAGTSSNVPVKQLAVASPPAMPDLLGDLMGIDNAIVPVDEPAAPSGPPLPVLLPSTTGQGLQISAQLTRRDGQIYYDISFENGTQGVLDGFMIQFNKNTFGLAAGEALQVTPLQPGQSTRTLLQMTPFQNISPGAPNSLLQVAVKNNQQPVWYFNDKIPLHVFFGEDGKMERAGFLEAWKSLPDDNEFTKEFPGSVISSIDATVERLVASNVFFIAKRKNANMDVLYLSAKMPRGIPFLIEVTAVVGVPGVKCAVKTPNREMVPLFFEAMEALTK

SEQ ID NO: 191 - DNA, 2258 bp, Oryza sativa
gcaccaggcaacatgcaaattgtgctgttgttctctctgctgtaaagataatccttttacaaatggagctcattactagcacagatgttgtccgtaatctctgtaagaaaatggcaccccctcttgttacgcttttgtcagcagaacctgagattcagtatgtagcgttaaggaacattaatctgattgttcagaaaaggcctactatacttgcacatgaaattaaggtcttcttttgcaagtataatgacccaatatatgttaagatggaaaagctggagattatgataaagcttgcctctgacagaaacatagatcaggttctattggaattcaaagagtatgccacagaggtggatgttgactttgtccgcaaagctgttcgtgctattggaagatgtgcaattaagttggagagagctgctgaaaggtgtatcagtgttttgcttgagctgattaagataaaggttaattatgttgtgcaggaggctataattgttattaaggacatcttcaggcgctaccctaatacgtatgagtcgatcatcgcaacactctgtgaaagcctagacaccttagatgaaccagaggctaaggcatcaatgatttggattattggagaatatgctgaaaggattgacaatgctgacgaacttcttgagagcttcttggaaacattcccagaagaaccagcattagttcaattgcagttactaacggcaactgttaagttgttccttaaaaagccaactgaggggcctcaacagatgatacaggctgttctcaataatgcaacagttgaaacagacaatcctgatttgcgcgaccgagcttatatatactggcgacttctttctactgatcctgaggcagctaaagatgtagttttggcagagaaacctgtgatcagcgatgattccaaccagcttgattcttctctcctagatgatctgctagccaatatttctaccctttcatcagtttatcacaagcctccagaagcatttgttagccgcgttaaaacagctcctagggctgatgatgaggagtttgctgatacagctgaaacaggatattcggagtcaccatctcagggtgttgatggggcatcaccttcctctagtgctggcacttcttctaatgttccagtgaagcagccagcagcaccagctgctcctgctccaatgccagacctccttggtgatttgatgggtatggataactccattgttcctgttgatgaaccaacagcaccttcaggccctccactacctgttttgttgccatcaaccactggccaaggactgcagatcagcgcacaactagtgcggcgtgatggccaaatattctatgatatatcttttgataatggcactcaaactgtgctagatggattcatgattcagtttaacaaaaatacctttggccttgcagccggtggtgcacttcaggtctctccactgcaacctgggacctcggccaggacgctgctacctatggtggcattccagaatctctctcctggagcgccaagctcactgctgcaggttgcggtgaagaataatcagcaacctgtgtggtacttcaatgacaaaatccctatgcatgccttctttggtgaagatggcaaaatggaacgaacaagttttcttgaggcctggaaatctttacctgatgacaacgaattttcgaaagagttcccctcttctgtcgtcagcagcatagatgcgaccgttgagcaccttgcagcatcaaatgtgttctttatcgccaagaggaaaaactcaaacaaggatgttctgtacatgtctgcaaagattccgcgtggaatccccttcctgatagagcttactgctgcagtcggtgttcctggcgtgaagtgtgcggtcaaaactccaaacaaggagatggtggctctcttcttcgaagccatggagtctcttctcaagtgatacaaaattgaaggatcattgttccttccaaattgatcagttcatgagctattgtaggtttggatgcggcgttgtttcacaggagctggtgtgaattgtatttgttgttctttgtattagattactgtatttaaactgctagtttcctggtttcaaagttttttcacgacgaacactcgacatggccattttgtctgcgtaatttgtatatctcggacctgaacctcttttcatgccatagcatatgaaatataagtagcttgcaaactcgtaaaaaaaaaaaaaaaaaaaaaaaaaaaaaaaaaa

SEQ ID NO: 192 - protein, 649 aa, Oryza sativa
TRQHANCAVVLSAVKIILLQMELITSTDVVRNLCKKMAPPLVTLLSAEPEIQYVALRNINLIVQKRPTILAHEIKVFFCKYNDPIYVKMEKLEIMIKLASDRNIDQVLLEFKEYATEVDVDFVRKAVRAIGRCAIKLERAAERCISVLLELIKIKVNYVVQEAIIVIKDIFRRYPNTYESIIATLCESLDTLDEPEAKASMIWIIGEYAERIDNADELLESFLETFPEEPALVQLQLLTATVKLFLKKPTEGPQQMIQAVLNNATVETDNPDLRDRAYIYWRLLSTDPEAAKDVVLAEKPVISDDSNQLDSSLLDDLLANISTLSSVYHKPPEAFVSRVKTAPRADDEEFADTAETGYSESPSQGVDGASPSSSAGTSSNVPVKQPAAPAAPAPMPDLLGDLMGMDNSIVPVDEPTAPSGPPLPVLLPSTTGQGLQISAQLVRRDGQIFYDISFDNGTQTVLDGFMIQFNKNTFGLAAGGALQVSPLQPGTSARTLLPMVAFQNLSPGAPSSLLQVAVKNNQQPVWYFNDKIPMHAFFGEDGKMERTSFLEAWKSLPDDNEFSKEFPSSVVSSIDATVEHLAASNVFFIAKRKNSNKDVLYMSAKIPRGIPFLIELTAAVGVPGVKCAVKTPNKEMVALFFEAMESLLK

SEQ ID NO: 193 - DNA, 1544 bp, Glycine max
ggcaccagaccacttgtaactcttgttgcttcagccccagaagtccaatatgttgctttgaggaatatcgaccttctcttgcaagcgaagccagacattctcagcaaagaactgagggtcttcttctgcaaatataatgatcccccatatgttaaacttcagaaattggagattatggtacggatagcaaatgacaagaatgttgatcaattgttatctgagctcaaagaatacgcacttgaagtcgatatggacttcgttcgaagagctgtcaaggctattggtcaagcagctataaaaattgagagcgcaagcgaaaagtgtgtcaacacattactcgatttgattgctacaaaggtcaactacgtggttcaagaggctattgtggttattaaagatattttcagaaagtatccaggttacgaaggaatcatcccgaccttgtgcaagtacattgacgaattagatgagccaaatgcaagaggagctttaatttggattgtcggcgagtatgctgagaagatcagcaacgcagatgaaattcttgcaggatttgttgaagggttcatggaagaatttacccaaactcaacttcaaatcctgacagctgtcgtcaagttatttttgaagaaaccggacaacaatcaaggtcttgttcaaaaggtcttgcaagtatcaacggccgaaaatgataaccctgatataagagatagagcttatgtatattggcgtttattatcaggtgatctttcaatagcaaaggacatcatcctttccgaaaagccacccatcactacaaccatgacatctcttccaccagctctcctcgaacaacttttaggtgaactcagcacgcttgcttctgtctaccataaaccccctgagacattcgttggccaaggtcgttacggcgctgacgccattcaacatgccgctatccaggaacaacgtcaaaatgctgtcgaaaacccaattgcggcggccgttgccgcagccgcaaatggtactactgctcaaaataatgctgagaatctcctcgatattgattttgatggtgcggctccagcaagtgcagatgcgcctccaactgcgggcacaagtggattggaaggattagcaggcacgcctcaaagagtagatagtccagctgcgggcgcaagtgcaggtggtaatatggcggatatgatgggtttatttgatgctcctattcctactgcaggcggaatgggtggcatgggtgggatgggaaatgatatgatgaatggattcgcagggttggatttgagtggttctagtcaaccacctggtgcacagacacagttacagcagggtggtgggaaaaaaacgacagaggatcttttgggaatgttttaggcttgaagatgatgttaagtcggtggggaaaagaggacgccggagcgggaagggtttgtggtagtcaaggaaagaggatagcgaaaatgaaatgtttgataagacagtcagtggttagaagttagtaaaatcattgattgattgattgattaaaaaaaaaaaaaaaaaaa

SEQ ID NO: 194 - protein, 457 aa, Glycine max
GTRPLVTLVASAPEVQYVALRNIDLLLQAKPDILSKELRVFFCKYNDPPYVKLQKLEIMVRIANDKNVDQLLSELKEYALEVDMDFVRRAVKAIGQAAIKIESASEKCVNTLLDLIATKVNYVVQEAIVVIKDIFRKYPGYEGIIPTLCKYIDELDEPNARGALIWIVGEYAEKISNADEILAGFVEGFMEEFTQTQLQILTAVVKLFLKKPDNNQGLVQKVLQVSTAENDNPDIRDRAYVYWRLLSGDLSIAKDIILSEKPPITTTMTSLPPALLEQLLGELSTLASVYHKPPETFVGQGRYGADAIQHAAIQEQRQNAVENPIAAAVAAAANGTTAQNNAENLLDIDFDGAAPASADAPPTAGTSGLEGLAGTPQRVDSPAAGASAGGNMADMMGLFDAPIPTAGGMGGMGGMGNDMMNGFAGLDLSGSSQPPGAQTQLQQGGGKKTTEDLLGMF

SEQ ID NO: 195 - DNA, 531 bp, Triticum aestivum
  unsure (442)
cagccaggacgctcctagctatggttttctcccagaatgtctctcctggagcaccaaactcgttactgcaggttgcagtgaagaataatcagcaacctgtgtggtatttcagcgacaaaggcttgctgcatgtgttctttggtgaagatggcaaaatggagcggacgagttttcttgaggcctggaaatctttacctgatgacaatgaattttcaaaagaataccccaattctgtcatcagcagcatagatgccaccgttgaacaccttgcagcatcaaatgtgttctttattgccaagcggaaaaatgcaaacatggatgtgctgtatctgtctgcgaaggtcccccgtgggattccatttctgatagagcttactgctgctgttggtgttcctgggtgccaagtgtgcagtcaagactccaaacaaggagttgtgcctcnatctttgaagctatggagtctcttacaattaagtgacgggaaacttgagatcgtatccgtcaaaattatccggtatgacataacaagcg

SEQ ID NO: 196 - protein, 116 aa, Triticum aestivum
PNSLLQVAVKNNQQPVWYFSDKGLLHVFFGEDGKMERTSFLEAWKSLPDDNEFSKEYPNSVISSIDATVEHLAASNVFFIAKRKNANMDVLYLSAKVPRGIPFLIELTAAVGVPGC